SEQUENCE LISTING (127 sequences)

SEQ ID NO: 1 - protein, 107 aa, synthetic construct
  REGION 1..107: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 2 - protein, 123 aa, synthetic construct
  REGION 1..123: note = source = /note="Description of Artificial Sequence: Synthetic polypeptide"
EVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSS

SEQ ID NO: 3 - protein, 107 aa, synthetic construct [sequence omitted]

SEQ ID NO: 4 - protein, 120 aa, synthetic construct
  REGION 1..120: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSS

SEQ ID NO: 5 - protein, 107 aa, synthetic construct
  REGION 1..107: note = source = /note="Description of Artificial Sequence: Synthetic polypeptide"
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 6 - protein, 118 aa, synthetic construct
  REGION 1..118: note = source = /note="Description of Artificial Sequence: Synthetic polypeptide"
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 7 - protein, 16 aa, synthetic construct
  REGION 1..16: note = source = /note="Description of Artificial Sequence: Synthetic peptide"
GTEGKSSGSGSESKST

SEQ ID NO: 8 - protein, 246 aa, synthetic construct
  REGION 1..246: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSS

SEQ ID NO: 9 - protein, 243 aa, synthetic construct
  REGION 1..243: note = source = /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSS

SEQ ID NO: 10 - protein, 241 aa, synthetic construct
  REGION 1..241: note = source = /note="Description of Artificial Sequence: Synthetic polypeptide"
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGTEGKSSGSGSESKSTQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 11 - protein, 19 aa, synthetic construct
  REGION 1..19: note = source = /note="Description of Artificial Sequence: Synthetic peptide"
MAWVWTLLFLMAAAQSIQA

SEQ ID NO: 12 - protein, 42 aa, synthetic construct
  REGION 1..42: note = source = /note="Description of Artificial Sequence: Synthetic polypeptide"
KRGRKKLLYIFKQPFMRPVQTTQEEDGCSCRFPEEEEGGCEL

SEQ ID NO: 13 - protein, 112 aa, synthetic construct
  REGION 1..112: note = source = /note="Description of Artificial Sequence: Synthetic polypeptide"
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 154 aa, synthetic construct
  REGION 1..154: note = source = /note="Description of Artificial Sequence: Synthetic polypeptide"
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 24 aa, synthetic construct
  REGION 1..24: note = source = /note="Description of Artificial Sequence: Synthetic peptide"
IYIWAPLAGTCGVLLLSLVITLYC

SEQ ID NO: 16 - protein, 45 aa, synthetic construct
  REGION 1..45: note = source = /note="Description of Artificial Sequence: Synthetic polypeptide"
TSTPAPRPPTPAPTIASQPLSLRPEACRPAAGGAVHTRGLDFACD

SEQ ID NO: 17 - protein, 265 aa, synthetic construct
  REGION 1..265: note = source = /note="Description of Artificial Sequence: Synthetic polypeptide"
MAWVWTLLFLMAAAQSIQAEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSS

SEQ ID NO: 18 - protein, 262 aa, synthetic construct
  REGION 1..262: note = source = /note="Description of Artificial Sequence: Synthetic polypeptide"
MAWVWTLLFLMAAAQSIQADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSS

SEQ ID NO: 19 - protein, 260 aa, synthetic construct
  REGION 1..260: note = source = /note="Description of Artificial Sequence: Synthetic polypeptide"
MAWVWTLLFLMAAAQSIQADIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGTEGKSSGSGSESKSTQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 20 - protein, 265 aa, synthetic construct
  REGION 1..265: note = source = /note="Description of Artificial Sequence: Synthetic polypeptide"
MAWVWTLLFLMAAAQSIQAEVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 21 - protein, 262 aa, synthetic construct
  REGION 1..262: note = source = /note="Description of Artificial Sequence: Synthetic polypeptide"
MAWVWTLLFLMAAAQSIQAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 22 - protein, 260 aa, synthetic construct
  REGION 1..260: note = source = /note="Description of Artificial Sequence: Synthetic polypeptide"
MAWVWTLLFLMAAAQSIQAQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGTEGKSSGSGSESKSTDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 23 - skipped

SEQ ID NO: 24 - protein, 246 aa, synthetic construct
  REGION 1..246: note = source = /note="Description of Artificial Sequence: Synthetic polypeptide"
EVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 25 - protein, 243 aa, synthetic construct
  REGION 1..243: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 26 - protein, 241 aa, synthetic construct
  REGION 1..241: note = source = /note="Description of Artificial Sequence: Synthetic polypeptide"
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGTEGKSSGSGSESKSTDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 27 - protein, 488 aa, synthetic construct
  REGION 1..488: note = source = /note="Description of Artificial Sequence: Synthetic polypeptide"
MAWVWTLLFLMAAAQSIQAEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKGTEGKSSGSGSESKSTEVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 485 aa, synthetic construct
  REGION 1..485: note = source = /note="Description of Artificial Sequence: Synthetic polypeptide"
MAWVWTLLFLMAAAQSIQADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGTEGKSSGSGSESKSTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 483 aa, synthetic construct
  REGION 1..483: note = source = /note="Description of Artificial Sequence: Synthetic polypeptide"
MAWVWTLLFLMAAAQSIQADIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGTEGKSSGSGSESKSTQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 488 aa, synthetic construct
  REGION 1..488: note = source = /note="Description of Artificial Sequence: Synthetic polypeptide"
MAWVWTLLFLMAAAQSIQAEVQLVQSGAEVKKPGESLKISCKGSGYSFTSYFIGWVRQMPGKGLEWMGIIYPGKSDTRYSPSFQGQVTISADKSISTAYLQWSSLKASDTAMYYCARVYSFGGRHKALFDYWGQGTLVTVSSGTEGKSSGSGSESKSTEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 485 aa, synthetic construct
  REGION 1..485: note = source = /note="Description of Artificial Sequence: Synthetic polypeptide"
MAWVWTLLFLMAAAQSIQAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESRWRGYKLDYWGQGTLVTVSSGTEGKSSGSGSESKSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 483 aa, synthetic construct
  REGION 1..483: note = source = /note="Description of Artificial Sequence: Synthetic polypeptide"
MAWVWTLLFLMAAAQSIQAQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGTEGKSSGSGSESKSTDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1407 bp, synthetic construct
  misc_feature 1..1407: note = source = /note="Description of Artificial Sequence: Synthetic polynucleotide"
  source 1..1407: mol_type = other DNA
gagatcgtgctgacccagagcccagccaccctgagcctgagcccaggcgagcgcgccaccctgagctgccgcgcctctcagagcgtgagcagctacctggcttggtatcagcagaagcccggacaggccccacgcctgctgatctacgacgccagcaaccgcgccaccggcatcccagcccgcttcagcggcagcggcagcggcaccgacttcaccctgaccatcagcagcctggagccagaggacttcgccgtgtactactgccagcagcgcagcaactggccactgaccttcggccagggcaccaaggtggagatcaagggtacagagggcaagtctagtggaagtggtagcgaaagtaagagtaccgaggtgcagctggtgcagagcggcgctgaggtcaaaaagccaggcgaaagccttaaaatatcatgcaaaggaagtggatattcctttaccagctacttcatcggctgggtgcgccagatgccaggcaagggcctggagtggatgggcatcatctacccaggcaagagcgacacccgctacagcccaagcttccagggccaggtgaccatcagcgccgacaagagcatcagcaccgcctacctgcagtggagcagcctgaaggccagcgacaccgccatgtactactgcgcccgcgtgtacagcttcggcggccgccacaaggccctgttcgactactggggccagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 34 - DNA, 1398 bp, synthetic construct
  misc_feature 1..1398: note = source = /note="Description of Artificial Sequence: Synthetic polynucleotide"
  source 1..1398: mol_type = other DNA
gacatccagatgacccagagcccaagcagcctgagcgccagcgtgggcgaccgcgtaaccattacttgtcgggcttcccaaagcattagtagctatttgaattggtatcaacaaaaaccaggcaaggccccaaagctgctgatctacgccgcctctagcctgcagagcggagtgccaagccgcttcagcggcagcggcagcggcaccgacttcaccctgaccatcagcagcctgcagccagaggacttcgccacctactactgccagcagagctacagcaccccactgaccttcggccagggcaccaaggtggagatcaagggtacagagggcaagtctagtggaagtggtagcgaaagtaagagtacccaggtgcagctggtgcagagcggcgccgaggtgaagaagccaggcagcagcgtgaaggtgagctgcaaggccagcggcggcaccttcagcagctacgccatcagctgggtgcgccaggccccaggccagggactggagtggatgggcggcatcatcccaatcttcggcaccgccaactacgcccagaagttccagggccgcgtgaccatcaccgccgacgagagcaccagcaccgcctacatggagctgagcagcctgcgcagcgaggacaccgccgtgtactactgcgcacgcgagagccgctggcgcggctacaagctggactactggggccagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 35 - DNA, 1392 bp, synthetic construct
  misc_feature 1..1392: note = source = /note="Description of Artificial Sequence: Synthetic polynucleotide"
  source 1..1392: mol_type = other DNA
gacatccagatgacccagagcccaagcagcctgagcgccagcgtgggcgaccgcgttacaataacttgtaaagcaagccaaaatgttgctactcacgtcggatggtatcagcaaaagccaggcaaggccccaaagcgcctgatctacagcgccagctaccgctacagcggagtgccaagccgcttcagcggcagcggcagcggcaccgagttcaccctgaccatcagcaacctgcagccagaggacttcgccacctactactgccagcagtacaaccgctacccatacaccttcggccagggcaccaagctggagatcaagggtacagagggcaagtctagtggaagtggtagcgaaagtaagagtacccaggtgcagctggtgcagagcggagccgaggtgaagaagccaggcgccagcgtgaaggtgagctgcaaggccagcggctacagcttcaccggctacaccatgaactgggtgcgccaggccccaggccagggactggagtggatgggcctgatcaacccatacaacagcgacaccaactacgcccagaagctgcagggccgcgtgaccatgaccaccgacaccagcaccagcaccgcctacatggagctgcgcagcctgcgcagcgacgacaccgccgtgtactactgcgcccgcgtggccctgcgcgtggccctggactactggggacagggcaccctggtgaccgtgagcagcactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 36 - DNA, 1407 bp, synthetic construct
  misc_feature 1..1407: note = source = /note="Description of Artificial Sequence: Synthetic polynucleotide"
  source 1..1407: mol_type = other DNA
gaggtgcagctggtgcagagcggcgctgaggtcaaaaagccaggcgaaagccttaaaatatcatgcaaaggaagtggatattcctttaccagctacttcatcggctgggtgcgccagatgccaggcaagggcctggagtggatgggcatcatctacccaggcaagagcgacacccgctacagcccaagcttccagggccaggtgaccatcagcgccgacaagagcatcagcaccgcctacctgcagtggagcagcctgaaggccagcgacaccgccatgtactactgcgcccgcgtgtacagcttcggcggccgccacaaggccctgttcgactactggggccagggcaccctggtgaccgtgagcagcggtacagagggcaagtctagtggaagtggtagcgaaagtaagagtaccgagatcgtgctgacccagagcccagccaccctgagcctgagcccaggcgagcgcgccaccctgagctgccgcgcctctcagagcgtgagcagctacctggcttggtatcagcagaagcccggacaggccccacgcctgctgatctacgacgccagcaaccgcgccaccggcatcccagcccgcttcagcggcagcggcagcggcaccgacttcaccctgaccatcagcagcctggagccagaggacttcgccgtgtactactgccagcagcgcagcaactggccactgaccttcggccagggcaccaaggtggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 37 - DNA, 1398 bp, synthetic construct
  misc_feature 1..1398: note = source = /note="Description of Artificial Sequence: Synthetic polynucleotide"
  source 1..1398: mol_type = other DNA
caggtgcagctggtgcagagcggcgccgaggtgaagaagccaggcagcagcgtgaaggtgagctgcaaggccagcggcggcaccttcagcagctacgccatcagctgggtgcgccaggccccaggccagggactggagtggatgggcggcatcatcccaatcttcggcaccgccaactacgcccagaagttccagggccgcgtgaccatcaccgccgacgagagcaccagcaccgcctacatggagctgagcagcctgcgcagcgaggacaccgccgtgtactactgcgcacgcgagagccgctggcgcggctacaagctggactactggggccagggcaccctggtgaccgtgagcagcggtacagagggcaagtctagtggaagtggtagcgaaagtaagagtaccgacatccagatgacccagagcccaagcagcctgagcgccagcgtgggcgaccgcgtaaccattacttgtcgggcttcccaaagcattagtagctatttgaattggtatcaacaaaaaccaggcaaggccccaaagctgctgatctacgccgcctctagcctgcagagcggagtgccaagccgcttcagcggcagcggcagcggcaccgacttcaccctgaccatcagcagcctgcagccagaggacttcgccacctactactgccagcagagctacagcaccccactgaccttcggccagggcaccaaggtggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 38 - DNA, 1392 bp, synthetic construct
  misc_feature 1..1392: note = source = /note="Description of Artificial Sequence: Synthetic polynucleotide"
  source 1..1392: mol_type = other DNA
caggtgcagctggtgcagagcggagccgaggtgaagaagccaggcgccagcgtgaaggtgagctgcaaggccagcggctacagcttcaccggctacaccatgaactgggtgcgccaggccccaggccagggactggagtggatgggcctgatcaacccatacaacagcgacaccaactacgcccagaagctgcagggccgcgtgaccatgaccaccgacaccagcaccagcaccgcctacatggagctgcgcagcctgcgcagcgacgacaccgccgtgtactactgcgcccgcgtggccctgcgcgtggccctggactactggggacagggcaccctggtgaccgtgagcagcggtacagagggcaagtctagtggaagtggtagcgaaagtaagagtaccgacatccagatgacccagagcccaagcagcctgagcgccagcgtgggcgaccgcgttacaataacttgtaaagcaagccaaaatgttgctactcacgtcggatggtatcagcaaaagccaggcaaggccccaaagcgcctgatctacagcgccagctaccgctacagcggagtgccaagccgcttcagcggcagcggcagcggcaccgagttcaccctgaccatcagcaacctgcagccagaggacttcgccacctactactgccagcagtacaaccgctacccatacaccttcggccagggcaccaagctggagatcaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgc

SEQ ID NO: 39 - protein, 7 aa, synthetic construct
  REGION 1..7: note = source = /note="Description of Artificial Sequence: Synthetic peptide"
GGTFSSY

SEQ ID NO: 40 - protein, 11 aa, synthetic construct
  REGION 1..11: note = source = /note="Description of Artificial Sequence: Synthetic peptide"
ESRWRGYKLDY

SEQ ID NO: 41 - protein, 6 aa, synthetic construct
  REGION 1..6: note = source = /note="Description of Artificial Sequence: Synthetic peptide"
IPIFGT

SEQ ID NO: 42 - protein, 7 aa, synthetic construct
  REGION 1..7: note = source = /note="Description of Artificial Sequence: Synthetic peptide"
GYSFTSY

SEQ ID NO: 43 - protein, 6 aa, synthetic construct
  REGION 1..6: note = source = /note="Description of Artificial Sequence: Synthetic peptide"
YPGKSD

SEQ ID NO: 44 - protein, 14 aa, synthetic construct
  REGION 1..14: note = source = /note="Description of Artificial Sequence: Synthetic peptide"
VYSFGGRHKALFDY

SEQ ID NO: 45 - protein, 7 aa, synthetic construct
  REGION 1..7: note = source = /note="Description of Artificial Sequence: Synthetic peptide"
GYSFTGY

SEQ ID NO: 46 - protein, 6 aa, synthetic construct
  REGION 1..6: note = source = /note="Description of Artificial Sequence: Synthetic peptide"
NPYNSD

SEQ ID NO: 47 - protein, 9 aa, synthetic construct
  REGION 1..9: note = source = /note="Description of Artificial Sequence: Synthetic peptide"
VALRVALDY

SEQ ID NO: 48 - protein, 11 aa, synthetic construct
  REGION 1..11: note = source = /note="Description of Artificial Sequence: Synthetic peptide"
RASQSISSYLN

SEQ ID NO: 49 - protein, 7 aa, synthetic construct
  REGION 1..7: note = source = /note="Description of Artificial Sequence: Synthetic peptide"
AASSLQS

SEQ ID NO: 50 - protein, 9 aa, synthetic construct
  REGION 1..9: note = source = /note="Description of Artificial Sequence: Synthetic peptide"
QQSYSTPLT

SEQ ID NO: 51 - protein, 11 aa, synthetic construct
  REGION 1..11: note = source = /note="Description of Artificial Sequence: Synthetic peptide"
RASQSVSSYLA

SEQ ID NO: 52 - protein, 7 aa, synthetic construct
  REGION 1..7: note = source = /note="Description of Artificial Sequence: Synthetic peptide"
DASNRAT

SEQ ID NO: 53 - protein, 9 aa, synthetic construct
  REGION 1..9: note = source = /note="Description of Artificial Sequence: Synthetic peptide"
QQRSNWPLT

SEQ ID NO: 54 - protein, 11 aa, synthetic construct
  REGION 1..11: note = source = /note="Description of Artificial Sequence: Synthetic peptide"
KASQNVATHVG

SEQ ID NO: 55 - protein, 7 aa, synthetic construct
  REGION 1..7: note = source = /note="Description of Artificial Sequence: Synthetic peptide"
SASYRYS

SEQ ID NO: 56 - protein, 9 aa, synthetic construct
  REGION 1..9: note = source = /note="Description of Artificial Sequence: Synthetic peptide"
QQYNRYPYT

SEQ ID NO: 57 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 58 - protein, 9 aa, synthetic construct
  REGION 1..9: note = source = /note="Description of Artificial Sequence: Synthetic peptide"
GGSLSSSSY

SEQ ID NO: 59 - protein, 5 aa, synthetic construct
  REGION 1..5: note = source = /note="Description of Artificial Sequence: Synthetic peptide"
YYSGN

SEQ ID NO: 60 - protein, 15 aa, synthetic construct
  REGION 1..15: note = source = /note="Description of Artificial Sequence: Synthetic peptide"
HVGYSYGRRFWYFDL

SEQ ID NO: 61 - protein, 11 aa, synthetic construct
  REGION 1..11: note = source = /note="Description of Artificial Sequence: Synthetic peptide"
RASQSVSSYLA

SEQ ID NO: 62 - protein, 7 aa, synthetic construct
  REGION 1..7: note = source = /note="Description of Artificial Sequence: Synthetic peptide"
DASNRAT

SEQ ID NO: 63 - protein, 9 aa, synthetic construct
  REGION 1..9: note = source = /note="Description of Artificial Sequence: Synthetic peptide"
QQRSNWPPT

SEQ ID NO: 64 - protein, 125 aa, synthetic construct
  REGION 1..125: note = source = /note="Description of Artificial Sequence: Synthetic polypeptide"
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSS

SEQ ID NO: 65 - protein, 107 aa, synthetic construct [sequence omitted]

SEQ ID NO: 66 - protein, 9 aa, synthetic construct
  REGION 1..9: note = source = /note="Description of Artificial Sequence: Synthetic peptide"
GFSLTNIRM

SEQ ID NO: 67 - protein, 5 aa, synthetic construct
  REGION 1..5: note = source = /note="Description of Artificial Sequence: Synthetic peptide"
FSNDE

SEQ ID NO: 68 - protein, 9 aa, synthetic construct
  REGION 1..9: note = source = /note="Description of Artificial Sequence: Synthetic peptide"
MRLPYGMDV

SEQ ID NO: 69 - protein, 16 aa, synthetic construct
  REGION 1..16: note = source = /note="Description of Artificial Sequence: Synthetic peptide"
RSSQSLVHSDGNTYLS

SEQ ID NO: 70 - protein, 7 aa, synthetic construct
  REGION 1..7: note = source = /note="Description of Artificial Sequence: Synthetic peptide"
KISNRFF

SEQ ID NO: 71 - protein, 9 aa, synthetic construct
  REGION 1..9: note = source = /note="Description of Artificial Sequence: Synthetic peptide"
MQATQFPHT

SEQ ID NO: 72 - protein, 119 aa, synthetic construct
  REGION 1..119: note = source = /note="Description of Artificial Sequence: Synthetic polypeptide"
QVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSS

SEQ ID NO: 73 - protein, 112 aa, synthetic construct
  REGION 1..112: note = source = /note="Description of Artificial Sequence: Synthetic polypeptide"
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIK

SEQ ID NO: 74 - protein, 20 aa, synthetic construct
  REGION 1..20: note = source = /note="Description of Artificial Sequence: Synthetic peptide"
GGSEGKSSGSGSESKSTGGS

SEQ ID NO: 75 - protein, 252 aa, synthetic construct
  REGION 1..252: note = source = /note="Description of Artificial Sequence: Synthetic polypeptide"
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSGGSEGKSSGSGSESKSTGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 76 - protein, 252 aa, synthetic construct
  REGION 1..252: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGGSEGKSSGSGSESKSTGGSQLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSS

SEQ ID NO: 77 - protein, 251 aa, synthetic construct
  REGION 1..251: note = source = /note="Description of Artificial Sequence: Synthetic polypeptide"
QVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSSGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIK

SEQ ID NO: 78 - protein, 251 aa, synthetic construct
  REGION 1..251: note = source = /note="Description of Artificial Sequence: Synthetic polypeptide"
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSS

SEQ ID NO: 79 - protein, 271 aa, synthetic construct
  REGION 1..271: note = source = /note="Description of Artificial Sequence: Synthetic polypeptide"
MAWVWTLLFLMAAAQSIQAQLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSGGSEGKSSGSGSESKSTGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 80 - protein, 271 aa, synthetic construct
  REGION 1..271: note = source = /note="Description of Artificial Sequence: Synthetic polypeptide"
MAWVWTLLFLMAAAQSIQAEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGGSEGKSSGSGSESKSTGGSQLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSS

SEQ ID NO: 81 - protein, 270 aa, synthetic construct
  REGION 1..270: note = source = /note="Description of Artificial Sequence: Synthetic polypeptide"
MAWVWTLLFLMAAAQSIQAQVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSSGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIK

SEQ ID NO: 82 - protein, 270 aa, synthetic construct
  REGION 1..270: note = source = /note="Description of Artificial Sequence: Synthetic polypeptide"
MAWVWTLLFLMAAAQSIQADIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSS

SEQ ID NO: 83 - protein, 475 aa, synthetic construct
  REGION 1..475: note = source = /note="Description of Artificial Sequence: Synthetic polypeptide"
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSGGSEGKSSGSGSESKSTGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 475 aa, synthetic construct
  REGION 1..475: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGGSEGKSSGSGSESKSTGGSQLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 474 aa, synthetic construct
  REGION 1..474: note = source = /note="Description of Artificial Sequence: Synthetic polypeptide"
QVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSSGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 474 aa, synthetic construct
  REGION 1..474: note = source = /note="Description of Artificial Sequence: Synthetic polypeptide"
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 1485 bp, synthetic construct
  misc_feature 1..1485: note = source = /note="Description of Artificial Sequence: Synthetic polynucleotide"
  source 1..1485: mol_type = other DNA
atggcttgggtgtggaccttgctattcctgatggcagctgcccaaagtatacaggcccaactccagctgcaagaatctggtcctggactcgtaaaaccatcagaaaccctctcattgacctgcacagtgagtggtggatcattgtcttccagcagctattggtgggggtggactcgacagccaccaggtcgcggtctcgagtggataggtacaatgtattacagtggtaacatttattacaaccctagcctccaaagccgggcaaccatctctgttgacacatccaagaatcaatttagcctgaaattgtctagtgtgactgctgctgatacagctgtttattattgcgctaggcatgtcggatactcttatggtcgtagattctggtacttcgatttgtggggtcgcgggaccttggtaacagtctcctccggaggatcagaggggaaatcttccggtagcggcagtgaatcaaagtcaactggtggttccgaaatcgtgctgactcagtcacccgcaactctttcactgagtcctggagaacgtgctactctgtcatgtcgggcttctcagtcagtaagttcttatttggcatggtaccagcaaaagcccggccaagccccccgactcttgatatacgatgcatcaaaccgtgccactggaatcccagcacggttttccggaagtggttccggaaccgacttcaccctcaccatatccagtttggagcccgaggacttcgcagtttactattgtcaacaacggtccaactggccccccacatttggacaaggcaccaaagtcgaaataaagactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgctga

SEQ ID NO: 88 - DNA, 1485 bp, synthetic construct
  misc_feature 1..1485: note = source = /note="Description of Artificial Sequence: Synthetic polynucleotide"
  source 1..1485: mol_type = other DNA
atggcttgggtgtggaccttgctattcctgatggcagctgcccaaagtatacaggccgaaatcgtactgactcaaagtcccgctactctcagtctgtcacccggcgagagagccacactgtcatgccgcgccagccaatcagtcagttcctaccttgcttggtatcagcagaaacctggccaagcacctcggctgcttatctacgacgccagcaatcgcgccactggtatcccagctcggttttcaggtagcggcagtgggacagactttaccttgactattagctctcttgaacccgaagactttgccgtttattactgccagcaacggtcaaactggcctcccacttttggccaaggaaccaaagtagagataaaaggtggttcagaaggtaaatcaagtgggtccggttccgaaagtaagtccaccggaggctctcaactccagttgcaggaaagtgggcctgggcttgtaaaaccaagcgagacattgtctctcacatgcactgtatcagggggatctctttcaagttcctcttattggtgggggtggactcgtcaaccccccggtaggggtctcgaatggatcggtaccatgtattattctgggaatatatactataatccaagccttcaaagtagagctactatatccgtggacacatccaagaatcagttctccttgaaactttctagcgtgaccgccgccgatactgctgtctactactgcgcacggcatgtgggatactcctacgggagacggttctggtatttcgacttgtggggtcgcggtacactcgttacagtgtcctctactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgctga

SEQ ID NO: 89 - DNA, 1482 bp, synthetic construct
  misc_feature 1..1482: note = source = /note="Description of Artificial Sequence: Synthetic polynucleotide"
  source 1..1482: mol_type = other DNA
atggcttgggtgtggaccttgctattcctgatggcagctgcccaaagtatacaggcccaagtaacactcaaggagagcggaccagtcttggtgaaaccaactgagaccttgactttgacatgtactgtaagtggcttcagccttaccaacatcaggatgtcagtatcttggataaggcaaccacctggcaaggcactcgaatggctggcacacatcttttctaacgacgaaaaatcctattcttccagtctcaaaagtcgccttaccatcagccgagataccagtaagagtcaagtagttcttacattgaccaatgtagatccagttgatacagccacatactactgcgcacgaatgcggcttccatacggcatggatgtatggggacagggaactactgttaccgttagttccggcggctccgagggcaagagcagcggcagcggcagcgagagcaagagcaccggcggcagcgacattgtgatgacccaaacacctcttagtagtcctgtaactctcggacagccagcttcaatatcttgtcgctcaagtcaatccctcgtccattccgacggcaacacctacctctcttggctccaacagagacccggccagcctcccagacttctcatctacaaaatcagtaacaggttcttcggcgtccctgacaggttcagtggatctggagcaggtacagatttcaccttgaagataagtagagtggaggctgaggacgtaggcgtctattattgtatgcaagctacccaattcccacatacattcggccaaggcactaaattggaaataaaaactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgctga

SEQ ID NO: 90 - DNA, 1482 bp, synthetic construct
  misc_feature 1..1482: note = source = /note="Description of Artificial Sequence: Synthetic polynucleotide"
  source 1..1482: mol_type = other DNA
atggcttgggtgtggaccttgctattcctgatggcagctgcccaaagtatacaggccgacattgtgatgacccaaacacctcttagtagtcctgtaactctcggacagccagcttcaatatcttgtcgctcaagtcaatccctcgtccattccgacggcaacacctacctctcttggctccaacagagacccggccagcctcccagacttctcatctacaaaatcagtaacaggttcttcggcgtccctgacaggttcagtggatctggagcaggtacagatttcaccttgaagataagtagagtggaggctgaggacgtaggcgtctattattgtatgcaagctacccaattcccacatacattcggccaaggcactaaattggaaataaaaggcggctccgagggcaagagcagcggcagcggcagcgagagcaagagcaccggcggcagccaagtaacactcaaggagagcggaccagtcttggtgaaaccaactgagaccttgactttgacatgtactgtaagtggcttcagccttaccaacatcaggatgtcagtatcttggataaggcaaccacctggcaaggcactcgaatggctggcacacatcttttctaacgacgaaaaatcctattcttccagtctcaaaagtcgccttaccatcagccgagataccagtaagagtcaagtagttcttacattgaccaatgtagatccagttgatacagccacatactactgcgcacgaatgcggcttccatacggcatggatgtatggggacagggaactactgttaccgttagttccactagtaccccagccccacgccctcccacccctgctcctacaatagcatcccagcccttgtcacttcgccccgaagcatgcagaccagccgcaggcggtgctgtgcatacccgaggactggacttcgcctgcgacatctacatctgggccccactggccggcacctgcggcgtgctgctgctgagcctggtgatcaccctgtactgcaagcgcggccgcaagaagctgctgtacatcttcaagcagccattcatgcgcccagtgcagaccacccaggaggaggacggctgcagctgccgcttcccagaggaggaggagggcggctgcgagctgcgcgtgaagttcagccgcagcgccgacgccccagcctacaagcagggccagaaccagctgtacaacgagctgaacctgggccgccgcgaggagtacgacgtgctggacaagcgccgcggccgcgacccagagatgggcggcaagccacgccgcaagaacccacaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgccaccacgctga

SEQ ID NO: 91 - protein, 20 aa, synthetic construct
  REGION 1..20: note = source = /note="Description of Artificial Sequence: Synthetic peptide"
GGSEGKSSGSGSESKSTGGS

SEQ ID NO: 92 - protein, 8 aa, synthetic construct
  REGION 1..8: note = source = /note="Description of Artificial Sequence: Synthetic peptide"
GGGSGGGS

SEQ ID NO: 93 - protein, 12 aa, synthetic construct
  REGION 1..12: note = source = /note="Description of Artificial Sequence: Synthetic peptide"
GGGSGGGSGGGS

SEQ ID NO: 94 - protein, 16 aa, synthetic construct
  REGION 1..16: note = source = /note="Description of Artificial Sequence: Synthetic peptide"
GGGSGGGSGGGSGGGS

SEQ ID NO: 95 - protein, 20 aa, synthetic construct
  REGION 1..20: note = source = /note="Description of Artificial Sequence: Synthetic peptide"
GGGSGGGSGGGSGGGSGGGS

SEQ ID NO: 96 - protein, 15 aa, synthetic construct
  REGION 1..15: note = source = /note="Description of Artificial Sequence: Synthetic peptide"
GGGGSGGGGSGGGGS

SEQ ID NO: 97 - protein, 20 aa, synthetic construct
  REGION 1..20: note = source = /note="Description of Artificial Sequence: Synthetic peptide"
GGGGSGGGGSGGGGSGGGGS

SEQ ID NO: 98 - protein, 25 aa, synthetic construct
  REGION 1..25: note = source = /note="Description of Artificial Sequence: Synthetic peptide"
GGGGSGGGGSGGGGSGGGGSGGGGS

SEQ ID NO: 99 - protein, 18 aa, synthetic construct
  REGION 1..18: note = source = /note="Description of Artificial Sequence: Synthetic peptide"
GSTSGSGKPGSGEGSTKG

SEQ ID NO: 100 - protein, 14 aa, synthetic construct
  REGION 1..14: note = source = /note="Description of Artificial Sequence: Synthetic peptide"
IRPRAIGGSKPRVA

SEQ ID NO: 101 - protein, 15 aa, synthetic construct
  REGION 1..15: note = source = /note="Description of Artificial Sequence: Synthetic peptide"
GKGGSGKGGSGKGGS

SEQ ID NO: 102 - protein, 15 aa, synthetic construct
  REGION 1..15: note = source = /note="Description of Artificial Sequence: Synthetic peptide"
GGKGSGGKGSGGKGS

SEQ ID NO: 103 - protein, 15 aa, synthetic construct
  REGION 1..15: note = source = /note="Description of Artificial Sequence: Synthetic peptide"
GGGKSGGGKSGGGKS

SEQ ID NO: 104 - protein, 15 aa, synthetic construct
  REGION 1..15: note = source = /note="Description of Artificial Sequence: Synthetic peptide"
GKGKSGKGKSGKGKS

SEQ ID NO: 105 - protein, 15 aa, synthetic construct
  REGION 1..15: note = source = /note="Description of Artificial Sequence: Synthetic peptide"
GGGKSGGKGSGKGGS

SEQ ID NO: 106 - protein, 15 aa, synthetic construct
  REGION 1..15: note = source = /note="Description of Artificial Sequence: Synthetic peptide"
GKPGSGKPGSGKPGS

SEQ ID NO: 107 - protein, 20 aa, synthetic construct
  REGION 1..20: note = source = /note="Description of Artificial Sequence: Synthetic peptide"
GKPGSGKPGSGKPGSGKPGS

SEQ ID NO: 108 - protein, 20 aa, synthetic construct
  REGION 1..20: note = source = /note="Description of Artificial Sequence: Synthetic peptide"
GKGKSGKGKSGKGKSGKGKS

SEQ ID NO: 109 - protein, 14 aa, synthetic construct
  REGION 1..14: note = source = /note="Description of Artificial Sequence: Synthetic peptide"
STAGDTHLGGEDFD

SEQ ID NO: 110 - protein, 15 aa, synthetic construct
  REGION 1..15: note = source = /note="Description of Artificial Sequence: Synthetic peptide"
GEGGSGEGGSGEGGS

SEQ ID NO: 111 - protein, 15 aa, synthetic construct
  REGION 1..15: note = source = /note="Description of Artificial Sequence: Synthetic peptide"
GGEGSGGEGSGGEGS

SEQ ID NO: 112 - protein, 15 aa, synthetic construct
  REGION 1..15: note = source = /note="Description of Artificial Sequence: Synthetic peptide"
GEGESGEGESGEGES

SEQ ID NO: 113 - protein, 15 aa, synthetic construct
  REGION 1..15: note = source = /note="Description of Artificial Sequence: Synthetic peptide"
GGGESGGEGSGEGGS

SEQ ID NO: 114 - protein, 20 aa, synthetic construct
  REGION 1..20: note = source = /note="Description of Artificial Sequence: Synthetic peptide"
GEGESGEGESGEGESGEGES

SEQ ID NO: 115 - protein, 18 aa, synthetic construct
  REGION 1..18: note = source = /note="Description of Artificial Sequence: Synthetic peptide"
GSTSGSGKPGSGEGSTKG

SEQ ID NO: 116 - protein, 19 aa, synthetic construct
  REGION 1..19: note = source = /note="Description of Artificial Sequence: Synthetic peptide"
PRGASKSGSASQTGSAPGS

SEQ ID NO: 117 - protein, 19 aa, synthetic construct
  REGION 1..19: note = source = /note="Description of Artificial Sequence: Synthetic peptide"
GTAAAGAGAAGGAAAGAAG

SEQ ID NO: 118 - protein, 19 aa, synthetic construct
  REGION 1..19: note = source = /note="Description of Artificial Sequence: Synthetic peptide"
GTSGSSGSGSGGSGSGGGG

SEQ ID NO: 119 - protein, 20 aa, synthetic construct
  REGION 1..20: note = source = /note="Description of Artificial Sequence: Synthetic peptide"
GKPGSGKPGSGKPGSGKPGS

SEQ ID NO: 120 - protein, 4 aa, synthetic construct
  REGION 1..4: note = source = /note="Description of Artificial Sequence: Synthetic peptide"
GSGS

SEQ ID NO: 121 - protein, 10 aa, synthetic construct
  REGION 1..10: note = source = /note="Description of Artificial Sequence: Synthetic peptide"
APAPAPAPAP

SEQ ID NO: 122 - protein, 20 aa, synthetic construct
  REGION 1..20: note = source = /note="Description of Artificial Sequence: Synthetic peptide"
APAPAPAPAPAPAPAPAPAP

SEQ ID NO: 123 - protein, 32 aa, synthetic construct
  REGION 1..32: note = source = /note="Description of Artificial Sequence: Synthetic polypeptide"
AEAAAKEAAAKEAAAAKEAAAAKEAAAAKAAA

SEQ ID NO: 124 - protein, 15 aa, synthetic construct
  REGION 1..15: note = source = /note="Description of Artificial Sequence: Synthetic peptide"
EPKSCDKTHTCPPCP

SEQ ID NO: 125 - protein, 12 aa, synthetic construct
  REGION 1..12: note = source = /note="Description of Artificial Sequence: Synthetic peptide"
ERKCCVECPPCP

SEQ ID NO: 126 - protein, 62 aa, synthetic construct
  REGION 1..62: note = source = /note="Description of Artificial Sequence: Synthetic polypeptide"
ELKTPLGDTTHTCPRCPEPKSCDTPPPCPRCPEPKSCDTPPPCPRCPEPKSCDTPPPCPRCP

SEQ ID NO: 127 - protein, 12 aa, synthetic construct
  REGION 1..12: note = source = /note="Description of Artificial Sequence: Synthetic peptide"
ESKYGPPCPSCP

(19) United States
(12) Patent Application Publication
(10) Pub. No.: US 2024/0262921 A1
(43) Pub. Date: Aug. 8, 2024

(54) GPRC5D CHIMERIC ANTIGEN RECEPTORS AND CELLS EXPRESSING THE SAME

(71) Applicant: Janssen Biotech, Inc., Horsham, PA (US)

(72) Inventors: Ricardo ATTAR, Lawrenceville, NJ (US); Rajkumar Ganesan, Blue Bell, PA (US); Francois Gaudet, Princeton, NJ (US); Bradley J. Heidrich, Gilbertsville, PA (US); Carmen Baca Jones, Ambler, PA (US); John Lee, North Wales, PA (US); Yingzhe Li, Dresher, PA (US); Sanjaya Singh, Blue Bell, PA (US); Sathya Venkataramani, Blue Bell, PA (US)

(21) Appl. No.: 18/643,356

(22) Filed: Apr. 23, 2024

Related U.S. Application Data
(62) Division of application No. 16/743,188, filed on Jan. 15, 2020.
(60) Provisional application No. 62/793,973, filed on Jan. 18, 2019.

(51) Int. Cl. C07K 16/28; A61K 9/00; A61K 39/00; A61P 35/00

(52) U.S. Cl. CPC C07K 16/2866; A61K 9/0019; A61P 35/00; A61K 2039/505; A61K 2039/54; A61K 2039/545; C07K 2317/24; C07K 2317/53; C07K 2317/565; C07K 2317/622; C07K 2317/73; C07K 2317/74; C07K 2319/02; C07K 2319/03; C07K 2319/30; C07K 2319/33

(57) ABSTRACT

The present disclosure provides for chimeric antigen receptors (CARs) that specifically target a G-protein coupled receptor, G-protein coupled receptor family C group 5 member D (GPRC5D), and immunoresponsive cells comprising such CARs, for the treatment of cancer.

### CROSS-REFERENCE TO RELATED APPLICATIONS

This application claims the benefit of U.S. Provisional Application Ser. No. 62/793,973, filed 18 Jan. 2019. The entire content of the aforementioned application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 9, 2020, is named JBI6043WOPCT1_SL.txt and is 150,002 bytes in size.

### TECHNICAL FIELD

The invention relates to GPRC5D-targeting chimeric antigen receptors (CARs) comprising GPRC5D single-chain variable fragments and engineered GPRC5D-targeting immune cells expressing the CARs. Also provided are nucleic acids and expression vectors encoding the CARs, recombinant cells containing the vectors, and compositions comprising the engineered immune cells expressing the GPRC5D-targeting CARs. Methods of making the CARs, and engineered immune cells, and methods of using the engineered immune cells to treat conditions including cancer are also provided.

### BACKGROUND

T cell therapy utilizes isolated T cells that have been genetically modified to enhance their specificity for a specific tumor associated antigen. Genetic modification may involve the expression of a chimeric antigen receptor (CAR) or an exogenous T cell receptor to provide new antigen specificity onto the T cell. T cells expressing chimeric antigen receptors (CAR-T cells) can induce tumor immunoreactivity. There is a need for better cancer therapies utilizing CAR-T cells.

### SUMMARY

Disclosed herein are chimeric antigen receptors (CARs), e.g., CARs that target a G-protein coupled receptor, G-protein coupled receptor family C group 5 member D (GPRC5D), cells comprising the CARs, vectors encoding the CARs, e.g., recombinant expression vectors, and nucleic acid molecules encoding the CARs, methods of making the CARs, compositions, polypeptides, proteins, nucleic acids, host cells, populations of cells and methods of treating disorders, e.g., cancer, using the disclosed CARs.

In one aspect, the disclosure provides a CAR, comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 66, a heavy chain CDR2 having the amino acid sequence of SEQ ID NO: 67, and a heavy chain CDR3 having the amino acid sequence of SEQ ID NO: 68;
a heavy chain complementarity determining region 1 (CDR1) having the amino acid sequence of SEQ ID NO: 58, a heavy chain CDR2 having the amino acid sequence of SEQ ID NO: 59, and a heavy chain CDR3 having the amino acid sequence of SEQ ID NO: 60;
a heavy chain complementarity determining region 1 (CDR1) having the amino acid sequence of SEQ ID NO: 39, a heavy chain CDR2 having the amino acid sequence of SEQ ID NO: 40, and a heavy chain CDR3 having the amino acid sequence of SEQ ID NO: 41;
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44; or
a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47;
wherein the extracellular antigen-binding domain binds the GPRC5D antigen.

In one embodiment:
the extracellular antigen-binding domain comprises the heavy chain CDR1 having the amino acid sequence of SEQ ID NO: 66, the heavy chain CDR2 having the amino acid sequence of SEQ ID NO: 67, and the heavy chain CDR3 having the amino acid sequence of SEQ ID NO: 68, and further comprises a light chain CDR1 having the amino acid sequence of SEQ ID NO: 69, a light chain CDR2 having the amino acid sequence of SEQ ID NO: 70, and a light chain CDR3 having the amino acid sequence of SEQ ID NO: 71;
the extracellular antigen-binding domain comprises the heavy chain CDR1 having the amino acid sequence of SEQ ID NO: 58, the heavy chain CDR2 having the amino acid sequence of SEQ ID NO: 59, and the heavy chain CDR3 having the amino acid sequence of SEQ ID NO: 60, and further comprises a light chain CDR1 having the amino acid sequence of SEQ ID NO: 61, a light chain CDR2 having the amino acid sequence of SEQ ID NO: 62, and a light chain CDR3 having the amino acid sequence of SEQ ID NO: 63
the extracellular antigen-binding domain comprises the heavy chain CDR1 having the amino acid sequence of SEQ ID NO: 39, the heavy chain CDR2 having the amino acid sequence of SEQ ID NO: 40, and the heavy chain CDR3 having the amino acid sequence of SEQ ID NO: 41, and further comprises a light chain CDR1 having the amino acid sequence of SEQ ID NO: 48, a light chain CDR2 having the amino acid sequence of SEQ ID NO: 49, and a light chain CDR3 having the amino acid sequence of SEQ ID NO: 50;
the extracellular antigen-binding domain comprises the heavy chain CDR1 having the amino acid sequence of SEQ ID NO: 42, the heavy chain CDR2 having the amino acid sequence of SEQ ID NO: 43, and the heavy chain CDR3 having the amino acid sequence of SEQ ID NO: 44, and further comprises a light chain CDR1 having the amino acid sequence of SEQ ID NO: 51, a light chain CDR2 having the amino acid sequence of SEQ ID NO: 52, and a light chain CDR3 having the amino acid sequence of SEQ ID NO: 53; or
the extracellular antigen-binding domain comprises the heavy chain CDR1 having the amino acid sequence of SEQ ID NO: 45, the heavy chain CDR2 having the amino acid sequence of SEQ ID NO: 46, and the heavy chain CDR3 having the amino acid sequence of SEQ ID NO: 47, and further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56.

In one embodiment, the extracellular antigen-binding domain comprises a light chain variable region (LCVR) comprising an amino acid sequence selected from the group consisting of SEQ ID NOS: 73, 65, 1, 3 and 5, or a heavy chain variable region (HCVR) comprising an amino acid sequence selected from the group consisting of SEQ ID NOS: 72, 64, 2, 4, and 6, or a combination of a LCVR comprising an amino acid sequence selected from the group consisting of SEQ ID NOS: 73, 65, 1, 3 and 5, and a HCVR comprising an amino acid sequence selected from the group consisting of SEQ ID NOS: 72, 64, 2, 4, and 6.

In one embodiment, the extracellular antigen-binding domain comprises:
a light chain variable region comprising an amino acid sequence of SEQ ID NO. 73 and a heavy chain variable region comprising an amino acid sequence of SEQ ID NO: 72;
a light chain variable region comprising an amino acid sequence of SEQ ID NO: 65 and a heavy chain variable region comprising an amino acid sequence of SEQ ID NO: 64;
a light chain variable region comprising an amino acid sequence of SEQ ID NO: 1 and a heavy chain variable region comprising an amino acid sequence of SEQ ID NO: 2;
a light chain variable region comprising an amino acid sequence of SEQ ID NO: 3 and a heavy chain variable region comprising an amino acid sequence of SEQ ID NO: 4; or
a light chain variable region comprising an amino acid sequence of SEQ ID NO: 5 and a heavy chain variable region comprising an amino acid sequence of SEQ ID NO: 6.

In one embodiment, the extracellular antigen-binding domain comprises a single-chain variable fragment (scFv). In some embodiments, the scFv comprises a linker polypeptide between the light chain variable region and the heavy chain variable region.

In one embodiment, the linker polypeptide comprises an amino acid sequence of SEQ ID NO: 7.

In one embodiment, the scFv comprises an amino acid sequence selected from the group consisting of SEQ ID NOS: 78, 77, 75, 76, 8, 9, 10, 24, 25, and 26.

In one embodiment, the extracellular antigen-binding domain comprises a signal polypeptide. In some embodiments, the signal polypeptide comprises an amino acid sequence of SEQ ID NO: 11.

In one embodiment, the intracellular signaling domain comprises a polypeptide component selected from the group consisting of a TNF receptor superfamily member 9 (CD137) component, a T-cell surface glycoprotein CD3 zeta chain (CD3z) component, a cluster of differentiation (CD27) component, a cluster of differentiation superfamily member (such as, e.g., CD28 or inducible T-cell co-stimulator (ICOS)) component, and a combination thereof.

In one embodiment, the CD137 component comprises an amino acid sequence of SEQ ID NO: 12.

In one embodiment, the CD3z component comprises an amino acid sequence of SEQ ID NO: 13.

In one embodiment, the intracellular signaling domain comprises an amino acid sequence of SEQ ID NO: 14.

In one embodiment, the transmembrane domain comprises a CD8a transmembrane region (CD8a-TM) polypeptide. In some embodiments, the CD8a-TM polypeptide comprises an amino acid sequence of SEQ ID NO. 15.

In one embodiment, the CAR further comprises a hinge region linking the transmembrane domain to the extracellular antigen-binding domain. In some embodiments, the hinge region is a CD8a-hinge region. In some embodiments, CD8a-hinge region comprises an amino acid sequence of SEQ ID NO: 16.

In one embodiment, the extracellular antigen-binding domain comprises an amino acid sequence selected from the group consisting of SEQ ID NOS: 82, 81, 80, 79, 17, 18, 19, 20, 21, and 22.

In one embodiment, a CAR of the present disclosure comprises an amino acid sequence selected from the group consisting of SEQ ID NOS: 86, 85, 83, 84, 27, 28, 29, 30, 31 and 32.

In one aspect, the disclosure provides isolated lymphocytes expressing the CARs as described herein. In some embodiments, the lymphocyte is a T lymphocyte. In some embodiments, the T lymphocyte is a naïve T cell. In some embodiments, the T lymphocyte is a memory stem T cell. In some embodiments, the T lymphocyte is a central memory T cell. In some embodiments, the T lymphocyte is CD4+. In some embodiments, the T lymphocyte is CD8+. In some embodiments, the T lymphocyte is CD4+ and CD8+.

In one aspect, the disclosure provides isolated nucleic acid molecules encoding any of the CARs described herein. In some embodiments, the nucleic acid molecule comprises a nucleic acid sequence selected from the group consisting of SEQ ID NOS: 90, 89, 87, 88, 33, 34, 35, 36, 37, and 38. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NOS: 90, 89, 87, 88, 33, 34, 35, 36, 37, or 38. In one embodiment, the present disclosure provides a vector comprising the nucleic acid molecule. In one embodiment, the present disclosure provides a cell expressing the nucleic acid molecule.

In one aspect, the present disclosure provides compositions, e.g., pharmaceutical compositions, comprising an effective amount of the lymphocyte expressing one or more of the CARs described and a pharmaceutically acceptable excipient.

In one aspect, the present disclosure provides a CAR according to the present disclosure for use in a method of therapy.

In one aspect, the present disclosure provides a lymphocyte according to the present disclosure for use in a method of therapy. In one aspect, the present disclosure provides a composition, e.g. a pharmaceutic composition, according to the present disclosure for use in a method of therapy.

In one aspect, the present disclosure provides a CAR according to the present disclosure for use in a method of treating cancer. In one aspect, the present disclosure provides a lymphocyte according to the present disclosure for use in a method of treating cancer. In one aspect, the present disclosure provides a composition, e.g. a pharmaceutic composition, according to the present disclosure for use in a method of treating cancer. In one embodiment, the cancer is selected from the group consisting of a bladder cancer, a metastatic bladder cancer, a esophageal cancer, a non-small-cell lung adenocarcinoma, a non-small cell lung squamous cell carcinoma, a prostate cancer, a urothelial carcinoma, a small cell lung cancer, an endometrial cancer, a cholangiocarcinoma, a hepatocellular carcinoma, sarcomas, solid tumors of squamous origin, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a non-Hodgkin's lymphoma (NHL), an acute lymphocytic leukemia (ALL), a chronic lymphocytic leukemia (CLL), a chronic myelogenous leukemia (CML), a smoldering multiple myeloma (SMM), a multiple myeloma (MM), an acute myeloid leukemia (AML), and combinations thereof. In one embodiment, the cancer being treated is multiple myeloma.

In one aspect, the present disclosure provides methods of treating a subject having cancer, the methods comprising administering a therapeutically effective amount of a lymphocyte expressing one or more of the CARs described to a subject in need thereof, whereby the lymphocyte induces killing of cancer cells in the subject. In one embodiment, the cancer is selected from the group consisting of a bladder cancer, a metastatic bladder cancer, a esophageal cancer, a non-small-cell lung adenocarcinoma, a non-small cell lung squamous cell carcinoma, a prostate cancer, a urothelial carcinoma, a small cell lung cancer, an endometrial cancer, a cholangiocarcinoma, a hepatocellular carcinoma, sarcomas, solid tumors of squamous origin, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, a non-Hodgkin's lymphoma (NHL), an acute lymphocytic leukemia (ALL), a chronic lymphocytic leukemia (CLL), a chronic myelogenous leukemia (CML), a smoldering multiple myeloma (SMM), a multiple myeloma (MM), an acute myeloid leukemia (AML), and combinations thereof. In one embodiment, the cancer being treated in a subject is multiple myeloma.

In one aspect, a method of targeted killing of a cancer cell is disclosed, the method comprising contacting the cancer cell with a lymphocyte expressing one or more of the CARs described, whereby the lymphocyte induces killing of the cancer cell. In some embodiments, the cancer cell is selected from the group consisting of a lung cancer cell, a gastric cancer cell, a colon cancer cell, a hepatocellular carcinoma cell, a renal cell carcinoma cell, a bladder urothelial carcinoma cell, a metastatic melanoma cell, a breast cancer cell, an ovarian cancer cell, a cervical cancer cell, a head and neck cancer cell, a pancreatic cancer cell, a glioma cell, a glioblastoma cell, and a non-Hodgkin's lymphoma (NHL) cell, an acute lymphocytic leukemia (ALL) cell, a chronic lymphocytic leukemia (CLL) cell, a chronic myelogenous leukemia (CML) cell, a smoldering multiple myeloma (SMM) cell, a multiple myeloma (MM) cell, an acute myeloid leukemia (AML) cell, and combinations thereof. In one embodiment, the cancer cell is a multiple myeloma cell.

### BRIEF DESCRIPTION OF THE DRAWINGS

The foregoing will be apparent from the following more particular description of example embodiments, as illustrated in the accompanying drawings.

FIG. 1 shows flow cytometric analyses of primary human pan T cells electroporated with no mRNA (mock) or 10 μg of mRNA expressing either an α-GPRC5D scFv CAR or isotype control CAR. 24 hours post-electroporation, CAR surface expression was measured by flow cytometry following stain with biotinylated L-protein and streptavidin-conjugated PE. Open histogram is mock, filled grey histogram is CAR-T population.

FIG. 2 shows flow cytometric analyses of transiently transfected pan T cells co-cultured with a multiple myeloma cell line. Twenty-four hours after transient transfection, primary pan T cells were labeled with Cell Trace Violet (CTV) fluorescent proliferation dye and then co-cultured with a multiple myeloma cell line, H929. Four days post co-culture, cells were pre-gated on the CD8+CD4− population and surface expression of activation markers CD25 and CD71 on CAR-T were compared to T cells cultured alone or in the presence of α-CD3/CD28 beads.

FIG. 3 shows a cytokine profile analysis for primary pan T cells transiently transfected with CAR-expressing mRNA and co-cultured with various myeloma cell lines. Primary pan T cells transiently transfected 24 hours prior with CAR-expressing mRNA were co-cultured at a 1:1 ratio with various myeloma cell lines expressing target antigen, GPRC5D, at high (+++), medium (++), low (+) or negative (−) levels. Sixteen hours post co-culture, supernatants were harvested and cytokine profile analysis by Meso Scale Discovery (MSD) of supernatants from co-cultures was performed.

FIG. 4 shows a cytokine profile analysis for primary pan T cells transiently transfected with CAR-expressing mRNA and co-cultured with autologous peripheral blood mononuclear cells (PBMCs) and a multiple myeloma cell line. Twenty-four hours after transient transfection with CAR-expressing mRNA, primary pan T cells were co-cultured (at a 1:1:1 ratio) with autologous PBMCs and the multiple myeloma cell line, H929. Sixteen hours post co-culture, supernatants were harvested. Cytokine profile analysis by MSD of supernatants from co-cultures containing all three populations (CAR-T, H929, autologous PBMC) were compared to control co-cultures lacking CAR-T cells, PBMC or H929, to each population of cells cultured in isolation.

FIG. 5 shows flow cytometric analyses of cytotoxic potential of CAR-T cells on GPRC5D-expressing myeloma cells. Primary pan T cells (transiently expressing one of three CARs as described previously) were co-cultured at the indicated Effector:Target (E:T) ratios with fluorescently labeled myeloma cell lines, MM1R, H929 and K562, for eight hours, at which time co-cultures were stained with viability dye. Percent killing is the ratio of the absolute number of live (viability dye negative) target (CTV positive) cells remaining in the co-culture relative to the number of live targets cultured without CAR-T cells.

FIG. 6 shows flow cytometric analyses of cytotoxic potential of CAR-T cells on GPRC5D-expressing myeloma cells. Primary multiple myeloma patient T cells (transiently expressing one of two CARs as described previously) were co-cultured at the indicated Effector:Target (E:T) ratios with fluorescently labeled myeloma cell line MM.1S or control GPRC5d-negative cell line KG-1 for 48 hours, at which time co-cultures were stained with viability dye. Percent cytotoxicity was determined as percent fluorescently-labeled cells that stained positive for viability dye, indicating cell death.

FIG. 7 shows results of flow cytometric analyses of the proliferative capacity of α-GPRC5D-CAR-T cells. Fluorescently labeled pan T cells (transiently expressing an α-GPRC5D CAR or isotype CAR control) were co-cultured at an E:T ratio of 1 with H929 for four days. Proliferation was measured as the absolute number of live (viability dye negative) CAR-T cells which had diluted fluorescent label (CTV). Proliferation in response to H929 was compared to CAR-T proliferation after four days of α-CD3/CD28 bead stimulation and CAR-T cultured alone (no stimulation control).

FIG. 8 shows a bar graph depicting the percentage of GPRC5D-CAR expression on healthy donor T cells, as measured by flow cytometry. The shaded gray bars represent background CAR detection in untransduced (mock) cells. The black bars show GPRC5D GC5B680-N68S-LH in transduced CAR-T cells. The values shown represent the mean±SD with 6 healthy donors.

FIGS. 9A-9F depict data showing expression of CD4, CD8, and memory markers on GPRC5D CAR-T cells. FIGS. 9A-9C show bar graphs, with the data collectively demonstrating that GPRC5D GC5B680-N68S-LH CAR was detected by flow cytometry in both CD4 and CD8 T cells, and that the CD4/CD8 proportion was not very different from an untransduced population. FIG. 9A shows the percentage of a mock (untransduced) cell population comprised by each of CD8⁺ and CD4⁺ cells. FIGS. 9B-9C each, respectively, show the percentage of CAR-cells (transduced cells not expressing GPRC5D CAR) comprised by each of CD8° and CD4⁺ cells and the percentage of CAR⁺ cells (transduced cells expressing GPRC5D CAR) comprised by each of CD8⁺ and CD4⁺ cells. FIG. 9D illustrates a flow cytogram prepared from one donor illustrating a gating strategy for selection of different indicated memory populations in a CAR-T population using surface markers (CD45RA and CD62L). FIGS. 9E and 9F depict bar plots showing that GPRC5D GC5B680-N68S-LH was expressed mostly on T_{N/SCM} and T_{CM}, which have been shown to have increased proliferative capacity, survival, and therapeutic efficacy. The values shown in FIGS. 9A-9C and 9E-9F represent the mean±SD with 6 healthy donors.

FIGS. 10A-10B show scatter plots demonstrating GPRC5D CAR-T toxicity. GPRC5D GC5B680-N68S-LH or untransduced (Mock) T cells were added at various E:T ratios to GPRC5D⁺ MM.1S cells or GPRC5D⁻ K562 cells containing a luciferase transgene. CAR-Ts were made from six healthy donors. The values are expressed as the mean±SD for 6 hours (FIG. 10A) or 24 hours (FIG. 10B). The percent specific lysis was calculated (i) by measuring luciferase signal in tumor cells in the presence of CAR-Ts at 6 or 24 hours divided by luciferase signal in tumor cells alone at the same time points and multiplied by 100 and (ii) by subtracting that number from 100. The equation is: 100−[(CAR-T+tumor luminescence/average tumor alone luminescence)×100]. The dotted line represents a percent specific lysis equal to zero. Negative lysis is indicative of cell growth.

FIGS. 11A-11F illustrate scatter plots demonstrating cytotoxicity of GPRC5D CAR-T cells added at indicated E:T ratios was assessed using GPRC5D⁺ H929, MM.1S, MM.1R, MOLP-2, and EJM cells or GPRC5D-K562 cells. The GPRC5D CAR-T cells showed cytotoxicity against all the GPRC5D⁺ cell lines.

FIG. 12 shows a bar graph depicting levels of surface CD25 expression (% CD25) by untransduced (mock) or GC5B680-N68S-LH cells in the presence of K562 cells at an E:T ratio of 3:1. CAR-Ts were made from six healthy donors. The data is expressed as the mean±SD.

FIG. 13 illustrates flow cytograms demonstrating that GC5B680-N68S-LH proliferates specifically in the presence of GPRC5D+ target cells (H929 or MM.1S) or a positive control (agonist CD3/CD28 beads) and not in the absence of target cells or in the presence of GPRC5D− cells (K562). Cell proliferation was measured using CELLTRACE Violet (CTV) (X-axis). CAR⁺ events were detected using an anti-idiotype antibody (Y-axis).

FIG. 14 shows a bar graph demonstrating that levels of cleaved caspase 3/7 levels in CAR-T (GC5B680-N68S-LH) cells incubated with the GPRC5D⁺ cell line H929 for 24 hours remain unchanged compared to controls (staurosporine, GPRC5D− K562 cells, or alone). E:T ratio 1:2 was used for H929 and K562 cells. CAR⁻ represents transduced cells not expressing GPRC5D CAR. CAR⁺ represents transduced cells expressing GPRC5D CAR.

FIG. 15 shows a graph demonstrating the effect of GPRC5D directed CAR-T cells on established MM.1S Human MM Xenografts in NSG-B2M Mice according to Example 15. GC5B680-N68S-LH, GC5B680-N68S-HL, GC5B83-N24T-N31S-LH, and GC5B83-N24T-N31S-HL, GPRC5D-directed CAR-Ts, Mock, untransduced CAR-Ts, PBS, Phosphate-buffered Saline, NSG, non-obese diabetic severe combined immunodeficiency gamma, B2M, Beta-2 microglobulin, MM, Multiple Myeloma. Group tumor volumes are graphed as mean±SEM. Tumor cells were implanted on Day 0, 1×10⁶ CAR+ T cells were implanted on Day 13. The symbol “*” denotes a significant difference on Day 26 vs. PBS control (p≤0.05, n=10/group).

FIG. 16 shows a graph demonstrating the effect of GC5B680-N68S-LH on Survival of NSG Mice Bearing Disseminated H929 human MM Xenografts according to Example 16. GC5B680-N68S-LH, GC5B680-N68S-HL, GC5B83-N24T-N31 S-LH, and GC5B83-N24T-N31S-HL, GPRC5D-directed CAR-T, Mock, untransduced CAR-Ts, PBS, Phosphate-buffered Saline, NSG, non-obese diabetic severe combined immunodeficiency gamma, MM, Multiple Myeloma. Tumor cells were implanted on Day 0; 1×10⁶ CAR+ T cells were implanted on Day 9. The symbol “*” denotes a significant difference on Day 111 vs. PBS control.

### DETAILED DESCRIPTION

A description of example embodiments follows.

The present disclosure provides for chimeric antigen receptors (CARs) that target a G-protein coupled receptor G-protein coupled receptor family C group 5 member D (GPRC5D), cells comprising such CARs, and methods of treating cancer (e.g., hematologic malignancies and solid tumors) using the CARs described herein.

The CARs of the invention have antigen specificity for GPRC5D (e.g., a human GPRC5D polypeptide having the amino acid sequence of SEQ ID NO: 57, or fragments thereof). The phrases “have antigen specificity” and “elicit antigen-specific response” as used herein mean that the CAR can specifically bind to and immunologically recognize an antigen, such that binding of the CAR to the GPRC5D antigen elicits an immune response. Methods of testing the CARs for antigen specificity and for the ability to recognize target cells are known in the art.

The disclosure also provides related nucleic acids, recombinant expression vectors, host cells, populations of cells, antibodies, or antigen binding portions thereof, and pharmaceutical compositions relating to the CARs of the invention.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting. As used herein, the indefinite articles “a”, “an” and “the” should be understood to include plural reference unless the context clearly indicates otherwise.

### Chimeric Antigen Receptors

The present invention relates generally to the use of T cells genetically modified to stably express a desired chimeric antigen receptor. A chimeric antigen receptor (CAR) is an artificially constructed hybrid protein or polypeptide containing the antigen binding domains of an antibody (scFv) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, CARs advantageously do not dimerize with endogenous T cell receptor (TCR) alpha and beta chains.

The CARs described herein provide recombinant polypeptide constructs comprising at least an extracellular antigen binding domain, a transmembrane domain and an intracellular signaling domain (also referred to herein as “a cytoplasmic signaling domain”) comprising a functional signaling domain derived from a stimulatory molecule as defined below. T cells expressing a CAR are referred to herein as CAR T cells, CAR-T cells or CAR modified T cells, and these terms are used interchangeably herein. The cell can be genetically modified to stably express an antibody binding domain on its surface, conferring novel antigen specificity that is MHC independent.

In some instances, the T cell is genetically modified to stably express a CAR that combines an antigen recognition domain of a specific antibody with an intracellular domain of the CD3-zeta chain or FcγRI protein into a single chimeric protein. In one embodiment, the stimulatory molecule is the zeta chain associated with the T cell receptor complex.

An “intracellular signaling domain,” as the term is used herein, refers to an intracellular portion of a molecule. It is the functional portion of the protein which acts by transmitting information within the cell to regulate cellular activity via defined signaling pathways by generating second messengers or functioning as effectors by responding to such messengers. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines.

In an embodiment, the intracellular signaling domain can comprise a primary intracellular signaling domain. Example primary intracellular signaling domains include those derived from the molecules responsible for primary stimulation, or antigen dependent simulation. In an embodiment, the intracellular signaling domain can comprise a costimulatory intracellular domain. Example costimulatory intracellular signaling domains include those derived from molecules responsible for costimulatory signals, or antigen independent stimulation. For example, in the case of a CAR-T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3-zeta, FcR gamma, FcR beta, CD3 gamma, CD3 delta, CD3 epsilon, CD5, CD22, CD79a, CD79b, and CD66d DAP10 and DAP12.

The primary intracellular signaling domain can be derived from the signaling domains of for example CD3-zeta, CD3 epsilon, CD22, CD79a, CD66d, CD39 DAP10, DAP12, Fc epsilon receptor I gamma chain (FCER1G), FcR beta, CD3 delta, CD3 gamma, CD5, CD226, or CD79B.

The term “zeta” or alternatively “zeta chain”, “CD3-zeta” or “TCR-zeta” is defined as the protein provided as GenBank Acc. No. BAG36664.1, or the equivalent residues from a non-human species, e.g., murine, rabbit, primate, mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

In a preferred embodiment, the intracellular signaling domain comprises a CD3-zeta stimulatory domain. In one aspect, the “zeta stimulatory domain” or a “CD3-zeta stimulatory domain” is the sequence provided as SEQ ID NO: 13.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as OX40, CD2, CD27, CD28, CDS, ICAM-1, LFA-1 (CD11a/CD18), 4-1BB (CD137), CD30, CD40, CD54 (ICAM), CD83, CD134 (OX40), CD150 (SLAMF1), CDJ152 (CTLA4), CD223 (LAG3), CD270 (HVEM), CD278 (ICOS), DAP10, LAT, NKD2C SLP76, TRIM, BTLA, GITR, CD226, HVEM, and ZAP70.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank accession no. AAA62478.2, or the equivalent residues from a non-human species, e.g., mouse, rodent, monkey, ape and the like. In one aspect, the “4-1BB costimulatory domain” is the sequence provided as SEQ ID NO: 12 or the equivalent residues from a non-human species, e.g., mouse, rodent, monkey, ape and the like.

In some embodiments, the cytoplasmic signaling domain further comprises one or more functional signaling domains derived from at least one costimulatory molecule as defined herein. In one embodiment, the costimulatory molecule is chosen from 4-1 BB (i.e., CD137), CD27, CD3-zeta and/or CD28. CD28 is a T cell marker important in T cell co-stimulation. CD27 is a member of the tumor necrosis factor receptor superfamily and acts as a co-stimulatory immune checkpoint molecule. 4-1BB transmits a potent costimulatory signal to T cells, promoting differentiation and enhancing long-term survival of T lymphocytes. CD3-zeta associates with TCRs to produce a signal and contains immunoreceptor tyrosine-based activation motifs (ITAMs).

In a preferred embodiment, the intracellular signaling domain comprises a costimulatory intracellular signaling domain, wherein the costimulatory intracellular signaling domain is a 4-1BB costimulatory domain. In a preferred embodiment the 4-1BB costimulatory domain is the sequence provided as SEQ ID NO: 12.

In one embodiment, a transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. In one example embodiment, the transmembrane domain is the CD8α transmembrane domain.

In one embodiment, the CAR comprises a hinge domain comprising a CD8α hinge domain.

In one embodiment, the CAR comprises a CD8α hinge domain and a CD8α transmembrane domain.

In one embodiment, the CAR comprises a hinge domain comprising a CD8α hinge domain and an intracellular signaling domain comprising CD28, 4-1BB, and CD3-zeta.

CARs described herein provide recombinant polypeptide constructs comprising at least an extracellular antigen binding domain, a transmembrane domain and an intracellular signaling domain (also referred to herein as “a cytoplasmic signaling domain”) comprising, e.g., a functional signaling domain derived from a stimulatory molecule as defined below

In one embodiment, the CAR comprises a chimeric fusion protein comprising an extracellular antigen recognition domain, a transmembrane domain and an intracellular signaling domain comprising a functional signaling domain derived from a stimulatory molecule. In one embodiment, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one embodiment, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule.

The CARs of the invention can be designed to comprise the CD28 and/or 4-1 BB signaling domain by itself or be combined with any other desired cytoplasmic domain(s) useful in the context of the CARs of the invention. In one embodiment, the cytoplasmic domain of the CAR can further comprise the signaling domain of CD3-zeta. For example, the cytoplasmic domain of the CAR can include but is not limited to CD3-zeta, 4-1BB and CD28 signaling modules and combinations thereof.

In a preferred embodiment, the CAR comprises a CD8α hinge domain, a CD8α transmembrane domain, and an intracellular signaling domain comprising the signaling domain of CD3-zeta.

In a preferred embodiment, the CAR comprises a CD8α hinge domain, a CD8α transmembrane domain, and an intracellular signaling domain comprising the signaling domain of CD3-zeta and a 4-1BB costimulatory domain.

Accordingly, the invention provides CAR T cells and methods of their use for adoptive therapy.

The disclosure further provides variants, e.g., functional variants, of the CARs, nucleic acids, polypeptides, and proteins described herein. “Variant” refers to a polypeptide or a polynucleotide that differs from a reference polypeptide or a reference polynucleotide by one or more modifications for example, substitutions, insertions or deletions. The term “functional variant” as used herein refers to a CAR, polypeptide, or protein having substantial or significant sequence identity or similarity to a parent CAR, polypeptide, or protein, which functional variant retains the biological activity of the CAR, polypeptide, or protein for which it is a variant. Functional variants encompass, e.g., those variants of the CAR, polypeptide, or protein described herein (the parent CAR, polypeptide, or protein) that retain the ability to recognize target cells to a similar extent, the same extent, or to a higher extent, as the parent CAR, polypeptide, or protein. In reference to the parent CAR, polypeptide, or protein, the functional variant can, for example, be at least about 30%, about 40%, about 50%, about 60%, about 75%, about 80%, about 85%, about 90%, about 91%, about 92%, about 93%, about 94%, about 95%, about 96%, about 97%, about 98%, about 99% or more identical in amino acid sequence to the parent CAR, polypeptide, or protein.

Herein, the structure of polypeptides is in places defined on the basis of % sequence identity with a recited reference sequence (with a given SEQ ID NO). In this context, % sequence identity between two amino acid sequences may be determined by comparing these two sequences aligned in an optimum manner and in which the amino acid sequence to be compared can comprise additions or deletions with respect to the reference sequence for an optimum alignment between these two sequences. The percentage of identity is calculated by determining the number of identical positions for which the amino acid residue is identical between the two sequences, by dividing this number of identical positions by the total number of positions in the comparison window and by multiplying the result obtained by 100 in order to obtain the percentage of identity between these two sequences. Typically, the comparison window with correspond to the full length of the sequence being compared. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/bl2.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program. Determining sequence identity of a query sequence to a reference sequence is within the ability of the skilled person and can be performed using commercially available analysis software such as BLAST™.

A functional variant can, for example, comprise the amino acid sequence of the parent CAR, polypeptide, or protein with at least one conservative amino acid substitution. In another embodiment, the functional variants can comprise the amino acid sequence of the parent CAR, polypeptide, or protein with at least one non-conservative amino acid substitution. In this case, the non-conservative amino acid substitution may not interfere with or inhibit the biological activity of the functional variant. The non-conservative amino acid substitution may enhance the biological activity of the functional variant such that the biological activity of the functional variant is increased as compared to the parent CAR, polypeptide, or protein.

Amino acid substitutions of the inventive CARs may be conservative amino acid substitutions. Conservative amino acid substitutions are known in the art, and include amino acid substitutions in which one amino acid having certain physical and/or chemical properties is exchanged for another amino acid that has the same or similar chemical or physical properties. For example, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gin, Ser, Thr, Tyr, etc.), etc.

The CAR, polypeptide, or protein can consist essentially of the specified amino acid sequence or sequences described herein, such that other components e.g., other amino acids, do not materially change the biological activity of the functional variant.

The CARs, polypeptides, and proteins of embodiments of the disclosure (including functional portions and functional variants) can be of any length, i.e., can comprise any number of amino acids, provided that the CARs, polypeptides, or proteins (or functional portions or functional variants thereof) retain their biological activity, e.g., the ability to specifically bind to an antigen, detect diseased cells (e.g., cancer cells) in a host, or treat or prevent disease in a host, etc. For example, the polypeptide can be about 50 to about 5000 amino acids long, such as about 50, about 70, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000 or more amino acids in length. The polypeptides of the invention also include oligopeptides.

The CARs, polypeptides, and proteins of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, α-(2-amino-2-norbornane)-carboxylic acid, α,γ-diaminobutyric acid, α,β-diaminopropionic acid, homophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, indoline-2-carboxylic acid, 1,2,3,4-tetrahydroisoquinoline-3-carboxylic acid, aminomalonic acid, aminomalonic acid monoamide, and α-tert-butylglycine.

The CARs, polypeptides, and proteins of embodiments of the invention (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The CARs, polypeptides, and/or proteins of embodiments of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the CARs, polypeptides, and proteins of the invention (including functional portions and functional variants thereof) can be isolated and/or purified from a source, such as a plant, a bacterium, an insect, a mammal, etc. Methods of isolation and purification are known in the art. Alternatively, the CARs, polypeptides, and/or proteins described herein (including functional portions and functional variants thereof) can be commercially synthesized. In this respect, the CARs, polypeptides, and proteins can be synthetic, recombinant, isolated, and/or purified.

Examples of modified nucleotides that can be used to generate the recombinant nucleic acids utilized to produce the polypeptides described herein include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N⁶-isopentenyladenine, uracil-5-oxyacetic acid (v), wybutoxosine, pseudouracil, queosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

The nucleic acid can comprise any isolated or purified nucleotide sequence which encodes any of the CARs, polypeptides, or proteins, or functional portions or functional variants thereof. Alternatively, the nucleotide sequence can comprise a nucleotide sequence which is degenerate to any of the sequences or a combination of degenerate sequences.

Some embodiments of the invention also provide an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

In an embodiment, the nucleic acids of the invention can be incorporated into a recombinant expression vector. The present disclosure provides recombinant expression vectors comprising any of the nucleic acids of the invention. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors of the invention are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence encoding the CAR, polypeptide, or protein (including functional portions and functional variants thereof), or to the nucleotide sequence which is complementary to or which hybridizes to the nucleotide sequence encoding the CAR, polypeptide, or protein. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Included in the scope of the invention are conjugates, e.g., bioconjugates, comprising any of the CARs, polypeptides, or proteins (including any of the functional portions or variants thereof), host cells, nucleic acids, recombinant expression vectors, populations of host cells, or 5 antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

An embodiment of the invention further provides an antibody, or antigen binding portion thereof, which binds, e.g., specifically binds, to an epitope of the CARs of the invention.

The antibody can be any type of immunoglobulin that is known in the art.

Immunoglobulins may be assigned to five major classes, IgA, IgD, IgE, IgG and IgM. IgA and IgG are further classified as the isotypes IgA1, IgA2, IgG1, IgG2, IgG3 and IgG4. Antibody light chains of vertebrate species can be assigned to one of two types, kappa (κ) and lambda (λ), based on the amino acid sequences of their constant domains. The antibody can be of any class or isotype.

The antibodies include immunoglobulin molecules including monoclonal antibodies including murine, human, humanized and chimeric monoclonal antibodies, polyclonal, antigen-binding fragments, bispecific or multispecific antibodies, monomeric, dimeric, tetrameric or multimeric antibodies, single chain antibodies, domain antibodies and any other modified configuration of the immunoglobulin molecule that comprises an antigen binding site of the required specificity. The antibody can be a naturally-occurring antibody, e.g., an antibody isolated and/or purified from a mammal, e.g., a murine, primate, mouse, rabbit, goat, horse, chicken, hamster, human, etc. Alternatively, the antibody can be an engineered (e.g., genetically-engineered) antibody.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

Typically, humanized antibodies have antigen binding sites derived from non-human species and the variable region frameworks are derived from human immunoglobulin sequences. Human antibodies have heavy and light chain variable regions in which both the framework and the antigen binding site are derived from sequences of human origin.

Also, the antibody can have any level of affinity or avidity for the functional portion of the CAR. In some embodiments, the antibody may bind the GPRC5D antigen with a range of affinities (K_{D}). In one embodiment according to the invention, and in some embodiments of each and every one of the numbered embodiments listed below, the antibody binds to the GPRC5D antigen with high affinity, for example, with a K_{D} equal to or less than about 10⁻⁷ M, such as but not limited to, 1-9.9 (or any range or value therein, such as 1, 2, 3, 4, 5, 6, 7, 8, or 9)×10⁻⁸M, 10⁻⁹M, 10⁻¹⁰M, 10⁻¹¹M, 10⁻¹²M, 10⁻¹³M, 10⁻¹⁴M, 10⁻¹⁵M or any range or value therein, as determined by surface plasmon resonance or the Kinexa method, as practiced by those of skill in the art. One example affinity is equal to or less than 1×10⁻⁸ M. Another example affinity is equal to or less than 1×10⁻⁹ M.

Methods of testing antibodies for the ability to bind to any functional portion of the CARs are known in the art and include any antibody-antigen binding assay, such as, for example, radioimmunoassay (RIA), Western blot, enzyme-linked immunosorbent assay (ELISA), immunoprecipitation, and competitive inhibition assays.

Suitable methods of making antibodies are known in the art. For instance, standard hybridoma methods are described in, e.g., Kohler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen (i.e., GPRC5D), and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are known in the art and are described in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

Antibodies, as utilized herein, can be multiple or single chain, or intact immunoglobulins, and may be derived from natural sources or from recombinant sources. Antibodies can be tetramers of immunoglobulin molecules.

In some embodiments, the antibody is a bispecific antibody. “Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens. The VL and/or the VH regions of existing antibodies or the VL and VH regions identified de novo as described herein may be engineered into bispecific full-length antibodies. Such bispecific antibodies may be made by modulating the CH3 interactions in antibody Fc to form bispecific antibodies using technologies such as those described in U.S. Pat. No. 7,695,936; Int. Pat. Publ. No. WO04/111233; U.S. Pat. Publ. No. U52010/0015133; U.S. Pat. Publ. No. US2007/0287170; Int. Pat. Publ. No. WO2008/119353; U.S. Pat. Publ. No. US2009/0182127; U.S. Pat. PubI. No. US52010/0286374; U.S. Pat. Publ. No. US2011/0123532; Int. Pat. Publ. No. WO2011/131746; Int. Pat. Publ. No. WO2011/143545; or U.S. Pat. Publ. No. US2012/0149876. For example, bispecific antibodies of the invention may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Example reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

The term “antibody fragment” refers to at least one portion of an intact antibody, or recombinant variants thereof, that retains the antigen binding properties of the parental full length antibody. It refers to, for example, the antigen binding domain, e.g., an antigenic determining variable region of an intact antibody, that is sufficient to confer recognition and binding, e.g., specific binding of the antibody fragment to a target, such as an antigen. “Antigen-binding fragment” refers to a portion of an immunoglobulin molecule Examples of antibody fragments include, but are not limited to, Fab, Fab′, F(ab′)₂, and Fv fragments, single chain antibodies (scFv), linear antibodies, single domain antibodies such as sdAb (either VL or VH), camelid VHH domains, and multi-specific antibodies formed from antibody fragments.

The term “scFv” refers to a protein comprising at least one antibody fragment comprising a variable region of a light chain and at least one antibody fragment comprising a variable region of a heavy chain. In some embodiments, the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An embodiment of the invention also provides antigen binding portions of any of the antibodies described herein. The antigen binding portion can be any portion that has at least one antigen binding site, such as Fab, F(ab′)₂, dsFv, sFv, diabodies, and triabodies.

In some embodiments, antigen-binding fragments are heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab′)₂, Fd and Fv fragments and domain antibodies (dAb) comprising (e.g., consisting of) either one VH domain or one VL domain. VH and VL domains may be linked together via a linker, e.g., a synthetic linker.

“Complementarity determining regions (CDR)” are antigen binding sites in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J Exp Med 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol Biol 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Also provided by the present disclosure is a nucleic acid comprising a nucleotide sequence encoding any of the CARs, polypeptides, or proteins described herein (including functional portions and functional variants thereof).

The portion of the CAR comprising an antibody or antibody fragment thereof may exist in a variety of forms where the antigen binding domain is expressed as part of a contiguous polypeptide chain including, for example, a single domain antibody fragment (sdAb), a scFv and a human chimeric or humanized antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In one aspect, the CAR comprises an antibody fragment that comprises a scFv.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and known in the art.

The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is apparent that the present disclosure includes, but is not limited to, the use of partial nucleotide sequences of more than one gene and that these nucleotide sequences are arranged in various combinations to encode polypeptides that elicit the desired immune response.

In one aspect, the disclosure provides a CAR, comprising an extracellular antigen-binding domain, a transmembrane domain and an intracellular signaling domain, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 66, a heavy chain CDR2 having the amino acid sequence of SEQ ID NO: 67, and a heavy chain CDR3 having the amino acid sequence of SEQ ID NO: 68;
a heavy chain complementarity determining region 1 (CDR1) having the amino acid sequence of SEQ ID NO: 58, a heavy chain CDR2 having the amino acid sequence of SEQ ID NO: 59, and a heavy chain CDR3 having the amino acid sequence of SEQ ID NO: 60;
a heavy chain complementarity determining region 1 (CDR1) having the amino acid sequence of SEQ ID NO: 39, a heavy chain CDR2 having the amino acid sequence of SEQ ID NO: 40, and a heavy chain CDR3 having the amino acid sequence of SEQ ID NO: 41;
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44; or
a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47;
   wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 66, a heavy chain CDR2 having the amino acid sequence of SEQ ID NO: 67, and a heavy chain CDR3 having the amino acid sequence of SEQ ID NO: 68; wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 58, a heavy chain CDR2 having the amino acid sequence of SEQ ID NO: 59, and a heavy chain CDR3 having the amino acid sequence of SEQ ID NO: 60; wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 39, a heavy chain CDR2 having the amino acid sequence of SEQ ID NO: 40, and a heavy chain CDR3 having the amino acid sequence of SEQ ID NO: 41; wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44 wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises: a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47;
wherein the extracellular antigen-binding domain binds the GPRC5D antigen.

In one embodiment, the extracellular antigen-binding domain comprises
the extracellular antigen-binding domain comprising the heavy chain CDR1 having the amino acid sequence of SEQ ID NO: 66, the heavy chain CDR2 having the amino acid sequence of SEQ ID NO: 67, and the heavy chain CDR3 having the amino acid sequence of SEQ ID NO: 68, and further comprises a light chain CDR1 having the amino acid sequence of SEQ ID NO: 69, a light chain CDR2 having the amino acid sequence of SEQ ID NO: 70, and a light chain CDR3 having the amino acid sequence of SEQ ID NO: 71;
the extracellular antigen-binding domain comprising the heavy chain CDR1 having the amino acid sequence of SEQ ID NO: 58, the heavy chain CDR2 having the amino acid sequence of SEQ ID NO: 59, and the heavy chain CDR3 having the amino acid sequence of SEQ ID NO: 60, and further comprises a light chain CDR1 having the amino acid sequence of SEQ ID NO: 61, a light chain CDR2 having the amino acid sequence of SEQ ID NO: 62, and a light chain CDR3 having the amino acid sequence of SEQ ID NO: 63
the heavy chain CDR1 having the amino acid sequence of SEQ ID NO: 39, the heavy chain CDR2 having the amino acid sequence of SEQ ID NO: 40, and the heavy chain CDR3 having the amino acid sequence of SEQ ID NO: 41, and further comprises a light chain CDR1 having the amino acid sequence of SEQ ID NO: 48, a light chain CDR2 having the amino acid sequence of SEQ ID NO: 49, and a light chain CDR3 having the amino acid sequence of SEQ ID NO: 50;
the extracellular antigen-binding domain comprises the heavy chain CDR1 having the amino acid sequence of SEQ ID NO: 42, the heavy chain CDR2 having the amino acid sequence of SEQ ID NO: 43, and the heavy chain CDR3 having the amino acid sequence of SEQ ID NO: 44, and further comprises a light chain CDR1 having the amino acid sequence of SEQ ID NO: 51, a light chain CDR2 having the amino acid sequence of SEQ ID NO: 52, and a light chain CDR3 having the amino acid sequence of SEQ ID NO: 53; or
the extracellular antigen-binding domain comprises the heavy chain CDR1 having the amino acid sequence of SEQ ID NO: 45, the heavy chain CDR2 having the amino acid sequence of SEQ ID NO: 46, and the heavy chain CDR3 having the amino acid sequence of SEQ ID NO: 47, and further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having an amino acid sequence selected from the group consisting of SEQ ID NO: 66, SEQ ID NO: 58, SEQ ID NO: 39, SEQ ID NO: 42, and SEQ ID NO: 45, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR2 having an amino acid sequence selected from the group consisting of SEQ ID NO: 67, SEQ ID NO: 59, SEQ ID NO: 40, SEQ ID NO: 43, and SEQ ID NO: 46, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR3 having an amino acid sequence selected from the group consisting of SEQ ID NO: 68, SEQ ID NO: 60, SEQ ID NO: 41, SEQ ID NO: 44, and SEQ ID NO: 47, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having an amino acid sequence selected from the group consisting of SEQ ID NO: 66, SEQ ID NO: 58, SEQ ID NO: 39, SEQ ID NO: 42, and SEQ ID NO: 45, and conservative modifications thereof;
a heavy chain CDR2 having an amino acid sequence selected from the group consisting of SEQ ID NO: 67, SEQ ID NO: 59, SEQ ID NO: 40, SEQ ID NO: 43, and SEQ ID NO: 46, and conservative modifications thereof; and
a heavy chain CDR3 having an amino acid sequence selected from the group consisting of SEQ ID NO: 68, SEQ ID NO: 60, SEQ ID NO: 41, SEQ ID NO: 44, and SEQ ID NO: 47, and conservative modifications thereof;
wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises;
a light chain CDR1 having an amino acid sequence selected from the group consisting of SEQ ID NO: 69, SEQ ID NO: 54, SEQ ID NO: 48, SEQ ID NO: 51, and SEQ ID NO: 54, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a light chain CDR2 having an amino acid sequence selected from the group consisting of SEQ ID NO: 70, SEQ ID NO: 55, SEQ ID NO: 49, SEQ ID NO: 52, and SEQ ID NO: 55, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a light chain CDR3 having an amino acid sequence selected from the group consisting of SEQ ID NO: 71, SEQ ID NO: 56, SEQ ID NO: 50, SEQ ID NO: 53, and SEQ ID NO: 56, and conservative modifications thereof, wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a light chain CDR1 having an amino acid sequence selected from the group consisting of SEQ ID NO: 69, SEQ ID NO: 54, SEQ ID NO: 48, SEQ ID NO: 51, and SEQ ID NO: 54, and conservative modifications thereof;
a light chain CDR2 having an amino acid sequence selected from the group consisting of SEQ ID NO: 70, SEQ ID NO: 55, SEQ ID NO: 49, SEQ ID NO: 52, and SEQ ID NO: 55, and conservative modifications thereof; and
a light chain CDR3 having an amino acid sequence selected from the group consisting of SEQ ID NO: 71, SEQ ID NO: 56, SEQ ID NO: 50, SEQ ID NO: 53, and SEQ ID NO: 56, and conservative modifications thereof; wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having an amino acid sequence selected from the group consisting of SEQ ID NO: 66, SEQ ID NO: 58, SEQ ID NO: 39, SEQ ID NO: 42, and SEQ ID NO: 45, and conservative modifications thereof;
a heavy chain CDR2 having an amino acid sequence selected from the group consisting of SEQ ID NO: 67, SEQ ID NO: 59, SEQ ID NO: 40, SEQ ID NO: 43, and SEQ ID NO: 46, and conservative modifications thereof; and
a heavy chain CDR3 having an amino acid sequence selected from the group consisting of SEQ ID NO: 68, SEQ ID NO: 60, SEQ ID NO: 41, SEQ ID NO: 44, and SEQ ID NO: 47, and conservative modifications thereof; and
a light chain CDR1 having an amino acid sequence selected from the group consisting of SEQ ID NO: 69, SEQ ID NO: 54, SEQ ID NO: 48, SEQ ID NO: 51, and SEQ ID NO: 54, and conservative modifications thereof;
a light chain CDR2 having an amino acid sequence selected from the group consisting of SEQ ID NO: 70, SEQ ID NO: 55, SEQ ID NO: 49, SEQ ID NO: 52, and SEQ ID NO: 55, and conservative modifications thereof; and
a light chain CDR3 having an amino acid sequence selected from the group consisting of SEQ ID NO: 71, SEQ ID NO: 56, SEQ ID NO: 50, SEQ ID NO: 53, and SEQ ID NO: 56, and conservative modifications thereof;
wherein the extracellular antigen-binding domain binds the GPRC5D antigen.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 66, a heavy chain CDR2 having the amino acid sequence of SEQ ID NO: 67, and a heavy chain CDR3 having the amino acid sequence of SEQ ID NO: 68, and further comprises a light chain CDR1 having the amino acid sequence of SEQ ID NO: 69, a light chain CDR2 having the amino acid sequence of SEQ ID NO: 70, and a light chain CDR3 having the amino acid sequence of SEQ ID NO: 71

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 58, a heavy chain CDR2 having the amino acid sequence of SEQ ID NO: 59, and a heavy chain CDR3 having the amino acid sequence of SEQ ID NO: 60, and further comprises a light chain CDR1 having the amino acid sequence of SEQ ID NO: 61, a light chain CDR2 having the amino acid sequence of SEQ ID NO: 62, and a light chain CDR3 having the amino acid sequence of SEQ ID NO: 63.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 39, a heavy chain CDR2 having the amino acid sequence of SEQ ID NO: 40, and a heavy chain CDR3 having the amino acid sequence of SEQ ID NO: 41, and further comprises a light chain CDR1 having the amino acid sequence of SEQ ID NO: 48, a light chain CDR2 having the amino acid sequence of SEQ ID NO: 49, and a light chain CDR3 having the amino acid sequence of SEQ ID NO: 50.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44, and further comprises a light chain CDR1 having the amino acid sequence of SEQ ID NO: 51, a light chain CDR2 having the amino acid sequence of SEQ ID NO: 52, and a light chain CDR3 having the amino acid sequence of SEQ ID NO: 53.

In one aspect, the disclosure provides a CAR, comprising an extracellular antigen-binding domain, a transmembrane domain and an intracellular signaling domain, wherein the extracellular antigen-binding domain comprises:
a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47, and further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56.

In one embodiment, the extracellular antigen-binding domain comprises a light chain variable region (LCVR) comprising an amino acid sequence selected from the group consisting of SEQ ID NOS: 73, 65, 1, 3 and 5, or a heavy chain variable region (HCVR) comprising an amino acid sequence selected from the group consisting of SEQ ID NOS: 72, 64, 2, 4, and 6, or a combination of a LCVR comprising an amino acid sequence selected from the group consisting of SEQ ID NOS: 73, 65, 1, 3 and 5, and a HCVR comprising an amino acid sequence selected from the group consisting of SEQ ID NOS: 72, 64, 2, 4, and 6.

In some embodiments, the extracellular antigen-binding domain comprises a light chain variable region (LCVR) comprising an amino acid sequence of any one of SEQ ID NOS: 73, 65, 1, 3 or 5. In some embodiments, the extracellular antigen-binding domain comprises a light chain variable region (LCVR) comprising an amino acid sequence having at least 50%, at least 55%, at least 60%6, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 73, 65, 1, 3 or 5. In some embodiments, the extracellular antigen-binding domain comprises a light chain variable region (LCVR) comprising an amino acid sequence of SEQ ID NO: 73. In some embodiments, the extracellular antigen-binding domain comprises a light chain variable region (LCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 73. In some embodiments, the extracellular antigen-binding domain comprises a light chain variable region (LCVR) comprising an amino acid sequence of SEQ ID NO: 65. In some embodiments, the extracellular antigen-binding domain comprises a light chain variable region (LCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 65. In some embodiments, the extracellular antigen-binding domain comprises a light chain variable region (LCVR) comprising an amino acid sequence of SEQ ID NO: 1. In some embodiments, the extracellular antigen-binding domain comprises a light chain variable region (LCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 1. In some embodiments, the extracellular antigen-binding domain comprises a light chain variable region (LCVR) comprising an amino acid sequence of SEQ ID NO: 3. In some embodiments, the extracellular antigen-binding domain comprises a light chain variable region (LCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 3. In some embodiments, the extracellular antigen-binding domain comprises a light chain variable region (LCVR) comprising an amino acid sequence of SEQ ID NO: 5. In some embodiments, the extracellular antigen-binding domain comprises a light chain variable region (LCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 5.

In some embodiments, the extracellular antigen-binding domain comprises a light chain variable region (LCVR) comprising an amino acid sequence of any one of SEQ ID NOS: 72, 64, 2, 4, or 6. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 72, 64, 2, 4, or 6. In some embodiments, the extracellular antigen-binding domain comprises a light chain variable region (LCVR) comprising an amino acid sequence of SEQ ID NO: 72. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 72. In some embodiments, the extracellular antigen-binding domain comprises a light chain variable region (LCVR) comprising an amino 5 acid sequence of SEQ ID NO: 2. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 64. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 2. In some embodiments, the extracellular antigen-binding domain comprises a light chain variable region (LCVR) comprising an amino acid sequence of SEQ ID NO: 6. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 4. In some embodiments, the extracellular antigen-binding domain comprises a heavy chain variable region (HCVR) comprising an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 6.

In one embodiment, the extracellular antigen-binding domain comprises: a light chain variable region comprising an amino acid sequence of SEQ ID NO: 73, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto, and a heavy chain variable region comprising an amino acid sequence of SEQ ID NO: 72, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto.

In one embodiment, the extracellular antigen-binding domain comprises: a light chain variable region comprising an amino acid sequence of SEQ ID NO: 65 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto, and a heavy chain variable region comprising an amino acid sequence of SEQ ID NO: 64 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto.

In one embodiment, the extracellular antigen-binding domain comprises: a light chain variable region comprising an amino acid sequence of SEQ ID NO: 1, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto, and a heavy chain variable region comprising an amino acid sequence of SEQ ID NO: 2, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto.

In one embodiment, the extracellular antigen-binding domain comprises: a light chain variable region comprising an amino acid sequence of SEQ ID NO: 3 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto, and a heavy chain variable region comprising an amino acid sequence of SEQ ID NO: 4, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto.

In one embodiment, the extracellular antigen-binding domain comprises: a light chain variable region comprising an amino acid sequence of SEQ ID NO: 5 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto, and a heavy chain variable region comprising an amino acid sequence of SEQ ID NO: 6 or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto.

In one embodiment, the extracellular antigen-binding domain comprises: a light chain variable region comprising an amino acid sequence of SEQ ID NO: 73 and a heavy chain variable region comprising an amino acid sequence of SEQ ID NO: 72.

In one embodiment, the extracellular antigen-binding domain comprises: a light chain variable region comprising an amino acid sequence of SEQ ID NO: 65 and a heavy chain variable region comprising an amino acid sequence of SEQ ID NO: 64.

In one embodiment, the extracellular antigen-binding domain comprises: a light chain variable region comprising an amino acid sequence of SEQ ID NO: 1 and a heavy chain variable region comprising an amino acid sequence of SEQ ID NO: 2.

In one embodiment, the extracellular antigen-binding domain comprises: a light chain variable region comprising an amino acid sequence of SEQ ID NO: 3 and a heavy chain variable region comprising an amino acid sequence of SEQ ID NO: 4.

In one embodiment, the extracellular antigen-binding domain comprises: a light chain variable region comprising an amino acid sequence of SEQ ID NO: 5 and a heavy chain variable region comprising an amino acid sequence of SEQ ID NO: 6.

Herein, in embodiments wherein the amino acid sequence of the VH domain exhibits less than 100% sequence identity with a given reference VH sequence may nevertheless comprise heavy chain CDRs which are identical to HCDR1, HCDR2 and HCDR3 of the reference sequence whilst exhibiting amino acid sequence variation within the framework regions. Likewise, embodiments wherein the amino acid sequence of the VL domain exhibits less than 100% sequence identity with a given reference sequence may nevertheless comprise light chain CDRs which are identical to LCDR1, LCDR2 and LCDR3 of the reference sequence whilst exhibiting amino acid sequence variation within the framework regions. Similarly, where a scFv or extracellular domain of a CAR of the disclosure exhibits less than 100% sequence identity with a given reference VH sequence may nevertheless comprise heavy chain CDRs which are identical to HCDR1, HCDR2 and HCDR3 of the reference sequence and light chain CDRs which are identical to LCDR1, LCDR2 and LCDR3 of the reference sequence, whilst exhibiting amino acid sequence variation within the framework regions.

In one embodiment, the extracellular antigen-binding domain comprises a scFv. In some embodiments, the scFv comprises a linker polypeptide between the light chain variable region and the heavy chain variable region. In certain embodiments, the extracellular antigen-binding domain is a scFv which comprises an amino acid sequence selected from the group consisting of SEQ ID NOS: 78, 77, 75, 76, 8, 9, 10, 24, 25, and 26, and specifically binds to a GPRC5D polypeptide (e.g., a human GPRC5D polypeptide having the amino acid sequence of SEQ ID NO. 57, or fragments thereof).

In some embodiments, the scFv comprises, from the N- to C-terminus, a VH, a linker and a VL (VH-Linker-VL).

In some embodiments, the scFv comprises, from the N- to C-terminus, the VL, the linker and the VH (VL-Linker-VH).

In recombinant expression systems, the linker is a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to GPRC5D.

The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some 5 embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

In one embodiment, the linker polypeptide comprises an amino acid sequence of SEQ ID NO: 7.

Other linker sequences may include portions of immunoglobulin hinge area, CL or CHI derived from any immunoglobulin heavy or light chain isotype. Exemplary linkers that may be used are shown in Table 1. Additional linkers are described for example in Int. Pat. Publ. No. WO2019/060695, incorporated by reference herein in its entirety.

In one embodiment, the linker polypeptide comprises an amino acid sequence of any one of SEQ ID NOs: 91-123.

TABLE 1

| | | SEQ |
|---|---|---|
| Linker | | ID |
| name | Amino acid sequence | NO: |
| Linker 1 | GGSEGKSSGSGSESKSTGGS | 91 |
| Linker 2 | GGGSGGGS | 92 |
| Linker 3 | GGGSGGGSGGGS | 93 |
| Linker 4 | GGGSGGGSGGGSGGGS | 94 |
| Linker 5 | GGGSGGGSGGGSGGGSGGGS | 95 |
| Linker 6 | GGGGSGGGGSGGGGS | 96 |
| Linker 7 | GGGGSGGGGSGGGGSGGGGS | 97 |
| Linker 8 | GGGGSGGGGSGGGGSGGGGSGGGGS | 98 |
| Linker 9 | GSTSGSGKPGSGEGSTKG | 99 |
| Linker 10 | IRPRAIGGSKPRVA | 100 |
| Linker 11 | GKGGSGKGGSGKGGS | 101 |
| Linker 12 | GGKGSGGKGSGGKGS | 102 |
| Linker 13 | GGGKSGGGKSGGGKS | 103 |
| Linker 14 | GKGKSGKGKSGKGKS | 104 |
| Linker 15 | GGGKSGGKGSGKGGS | 105 |
| Linker 16 | GKPGSGKPGSGKPGS | 106 |
| Linker 17 | GKPGSGKPGSGKPGSGKPGS | 107 |
| Linker 18 | GKGKSGKGKSGKGKSGKGKS | 108 |
| Linker 19 | STAGDTHLGGEDFD | 109 |
| Linker 20 | GEGGSGEGGSGEGGS | 110 |
| Linker 21 | GGEGSGGEGSGGEGS | 111 |
| Linker 22 | GEGESGEGESGEGES | 112 |
| Linker 23 | GGGESGGEGSGEGGS | 113 |
| Linker 24 | GEGESGEGESGEGESGEGES | 114 |
| Linker 25 | GSTSGSGKPGSGEGSTKG | 115 |
| Linker 26 | PRGASKSGSASQTGSAPGS | 116 |
| Linker 27 | GTAAAGAGAAGGAAAGAAG | 117 |
| Linker 28 | GTSGSSGSGSGGSGSGGGG | 118 |
| Linker 29 | GKPGSGKPGSGKPGSGKPGS | 119 |
| Linker 30 | GSGS | 120 |
| Linker 31 | APAPAPAPAP | 121 |
| Linker 32 | APAPAPAPAPAPAPAPAPAP | 122 |
| Linker 33 | AEAAAKEAAAKEAAAAKEAAAAKEAAAAKAAA | 123 |

In one embodiment, the scFv comprises an amino acid sequence selected from the group consisting of SEQ ID NOS: 78, 77, 75, 76, 8, 9, 10, 24, 25, and 26. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 78, 77, 75, 76, 8, 9, 10, 24, 25, and 26. In some embodiments, the scFv comprises an amino acid sequence of SEQ ID NO:78. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 78. In some embodiments, the scFv comprises an amino acid sequence of SEQ ID NO:77. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 77. In some embodiments, the scFv comprises an amino acid sequence of SEQ ID NO:75. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 75. In some embodiments, the scFv comprises an amino acid sequence of SEQ ID NO:76. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 76. In some embodiments, the scFv comprises an amino acid sequence of SEQ ID NO: 8. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 8. In some embodiments, the scFv comprises an amino acid sequence of SEQ ID NO: 9. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 9. In some embodiments, the scFv comprises an amino acid sequence of SEQ ID NO:10. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 10. In some embodiments, the scFv comprises an amino acid sequence of SEQ ID NO: 24. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 24. In some embodiments, the scFv comprises an amino acid sequence of SEQ ID NO:25. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 25. In some embodiments, the scFv comprises an amino acid sequence of SEQ ID NO: 26. In some embodiments, the scFv comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 26.

In one embodiment, the extracellular antigen-binding domain comprises a signal polypeptide. In some embodiments, the signal polypeptide comprises an amino acid sequence of SEQ ID NO: 11. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 11.

In one aspect, the disclosure provides a CAR, comprising an extracellular antigen-binding domain, a transmembrane domain and an intracellular signaling domain, wherein the extracellular antigen-binding domain comprises an amino acid sequence selected from the group consisting of SEQ ID NOS: 82, 81, 80, 79, 17, 18, 19, 20, 21, and 22, or a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity thereto. Another feature of the CAR having an extracellular antigen-binding domain comprising an amino acid sequence selected from the group consisting of SEQ ID NOS: 82, 81, 80, 79, 17, 18, 19, 20, 21, and 22 is that the extracellular antigen-binding domain binds the GPRC5D antigen.

In one embodiment, the intracellular signaling domain comprises a polypeptide component selected from the group consisting of a TNF receptor superfamily member 9 (CD137) component, a T-cell surface glycoprotein CD3 zeta chain (CD3z) component, a cluster of differentiation (CD27) component, a cluster of differentiation superfamily member (such as, e.g., CD28 or inducible T-cell co-stimulator (ICOS)) component, and a combination thereof.

In one embodiment, the CD137 component comprises an amino acid sequence of SEQ ID NO: 12. In some embodiments, the CD137 component comprises an amino acid sequence having at least 50%, at least 55%, at least 60%, at least 65%, at least 70%, at least 75%, at least 80%, at least 85%, at least 90%, at least 95%, at least 98% or at least 99%, sequence identity with SEQ ID NO: 12.

In one embodiment, the CD3z component comprises an amino acid sequence of SEQ ID NO: 13. In some embodiments, the CD3z component comprises an amino acid sequence having at least 50%, at least 55%, at least 60%, at least 65%, at least 70%, at least 75%, at least 80%, at least 85%, at least 90%, at least 95%, at least 98% or at least 99%, sequence identity with SEQ ID NO: 13.

In some embodiments, the intracellular signaling domain comprises a CD137 component and a CD3z component.

In one embodiment, the intracellular signaling domain comprises an amino acid sequence of SEQ ID NO: 14. In some embodiments, the intracellular signaling domain comprises an amino acid sequence having at least 50%, at least 55%, at least 60%, at least 65%, at least 70%, at least 75%, at least 80%, at least 85%, at least 90%, at least 95%, at least 98% or at least 99%, sequence identity with SEQ ID NO: 14.

The transmembrane domain of the CAR may be derived from the transmembrane domain of CD8, an α, β or ζ chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI la, CD18), ICOS (CD278), 4-1 BB (CD137), 4-1 BBL, GITR, CD40, BAFFR, HVEM (LIGHTR), SLAMF7, NKp80 (KLRFI), CD160, CD1 9, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI la, LFA-1, ITGAM, CDI 1b, ITGAX, CDI 1c, ITGB1, CD29, ITGB2, CDI 8, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In one embodiment, the transmembrane domain comprises a CD8α transmembrane region (CD8a-TM) polypeptide. In some embodiments, the CD8a-TM polypeptide comprises an amino acid sequence of SEQ ID NO: 15. In some embodiments, the CD8a-TM polypeptide comprises an amino acid sequence having at least 50%, at least 55%, at least 60%, at least 65%, at least 70%, at least 75%, at least 80%, at least 85%, at least 90%, at least 95%, at least 98% or at least 99%, sequence identity with SEQ ID NO: 15.

In one embodiment, the transmembrane domain comprises at least the transmembrane region(s) of the α, β or ζ chain of the T-cell receptor, CD28, CD3 epsilon, CD45, CD4, CD5, CD8, CD8α, CD9, CD16, CD22, CD33, CD37, CD40, CD64, CD80, CD86, CD134, CD137, or CD154. In another embodiment, the transmembrane domain comprises at least the transmembrane domain of ζ, q or FcεRIγ and -β, MB1 (Igα.), B29 or CD3-γ, ζ, or η. In another embodiment, the transmembrane domain is synthetic, e.g., comprising predominantly hydrophobic residues such as leucine and valine, a triplet of phenylalanine, or tryptophan.

In one embodiment, the CAR further comprises a hinge region linking the transmembrane domain to the extracellular antigen-binding domain. In some embodiments, the hinge region is a CD8a-hinge region. In some embodiments, CD8a-hinge region comprises an amino acid sequence of SEQ ID NO: 16. In some embodiments, the CD8a-hinge region comprises an amino acid sequence having at least 50, at least 55, at least 60, at least 65, at least 70, at least 75, at least 80, at least 85, at least 90, at least 95, at least 98 or at least 99%, sequence identity with SEQ ID NO: 16. In some embodiments, the hinge region comprises the sequence EPKSCDKTHTCPPCP (SEQ ID NO: 124), or comprises an amino acid sequence having at least 50, at least 55, at least 60, at least 65, at least 70, at least 75, at least 80, at least 85, at least 90, at least 95, at least 98 or at least 99%, sequence identity with EPKSCDKTHTCPPCP (SEQ ID NO: 124). In some embodiments, the hinge region comprises the sequence ERKCCVECPPCP (SEQ ID NO: 125), or comprises an amino acid sequence having at least 50, at least 55, at least 60, at least 65, at least 70, at least 75, at least 80, at least 85, at least 90, at least 95, at least 98 or at least 99%, sequence identity with ERKCCVECPPCP (SEQ ID NO: 125). In some embodiments, the hinge region comprises the sequence ELKTPLGDTTHTCPRCP(EPKSCDTPPPCPRCP)₃ SEQ ID NO: 126), or comprises an amino acid sequence having at least 50, at least 55, at least 60, at least 65, at least 70, at least 75, at least 80, at least 85, at least 90, at least 95, at least 98 or at least 99%, sequence identity with ELKTPLGDTTHTCPRCP(EPKSCDTPPPCPRCP)₃ SEQ ID NO: 126). In some embodiments, the hinge region comprises the sequence ESKYGPPCPSCP (SEQ ID NO: 127), or comprises an amino acid sequence having at least 50, at least 55, at least 60, at least 65, at least 70, at least 75, at least 80, at least 85, at least 90, at least 95, at least 98 or at least 99%, sequence identity with ESKYGPPCPSCP (SEQ ID NO: 127).

In one embodiment, the CAR comprises an extracellular antigen-binding domain, a hinge region, a transmembrane domain and an intracellular signaling domain. In one such embodiment, the hinge region is a CD8α hinge region, the transmembrane domain is a CD8a-TM domain, and the intracellular signaling domain comprises a CD3-zeta domain and a 4-1BB/CD137 domain. In one such embodiment, the hinge region is a CD8α hinge region comprising the amino acid sequence of SEQ ID No: 16, the transmembrane domain is a CD8a-TM domain comprising the amino acid sequence of SEQ ID NO: 15, and the intracellular signaling domain comprises a CD3-zeta domain and a 4-1BB/CD137 domain, wherein the intracellular signaling domain comprises the amino acid sequence of SEQ ID No: 14.

In one embodiment, the extracellular antigen-binding domain comprises an amino acid sequence selected from the group consisting of SEQ ID NOS: 82, 81, 80, 79, 17, 18, 19, 20, 21, and 22. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 82, 81, 80, 79, 17, 18, 19, 20, 21, and 22.

In some embodiments, the extracellular antigen-binding domain comprises an amino acid sequence of SEQ ID NO: 82. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 82.

In some embodiments, the extracellular antigen-binding domain comprises an amino acid sequence of SEQ ID NO: 81. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 81.

In some embodiments, the extracellular antigen-binding domain comprises an amino acid sequence of SEQ ID NO: 80. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 80.

In some embodiments, the extracellular antigen-binding domain comprises an amino acid sequence of SEQ ID NO: 79. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 79.

In some embodiments, the extracellular antigen-binding domain comprises an amino acid sequence of SEQ ID NO: 17. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 17.

In some embodiments, the extracellular antigen-binding domain comprises an amino acid sequence of SEQ ID NO: 18. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 18.

In some embodiments, the extracellular antigen-binding domain comprises an amino acid sequence of SEQ ID NO: 19. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 19.

In some embodiments, the extracellular antigen-binding domain comprises an amino acid sequence of SEQ ID NO: 20. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 20.

In some embodiments, the extracellular antigen-binding domain comprises an amino acid sequence of SEQ ID NO: 21. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 21.

In some embodiments, the extracellular antigen-binding domain comprises an amino acid sequence of SEQ ID NO: 22. In some embodiments, the extracellular antigen-binding domain comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 22.

In one embodiment, a CAR of the present disclosure comprises an amino acid sequence selected from the group consisting of SEQ ID NOS: 86, 85, 83, 84, 27, 28, 29, 30, 31 and 32. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 86, 85, 83, 84, 27, 28, 29, 30, 31 and 32.

In some embodiments, the CAR of the present disclosure comprises an amino acid sequence of SEQ ID NO: 86. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 86.

In some embodiments, the CAR of the present disclosure comprises an amino acid sequence of SEQ ID NO: 85. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 85.

In some embodiments, the CAR of the present disclosure comprises an amino acid sequence of SEQ ID NO: 83. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 83.

In some embodiments, the CAR of the present disclosure comprises an amino acid sequence of SEQ ID NO: 84. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 84.

In some embodiments, the CAR of the present disclosure comprises an amino acid sequence of SEQ ID NO: 27. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 27.

In some embodiments, the CAR of the present disclosure comprises an amino acid sequence of SEQ ID NO: 28. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 28.

In some embodiments, the CAR of the present disclosure comprises an amino acid sequence of SEQ ID NO: 29. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 29.

In some embodiments, the CAR of the present disclosure comprises an amino acid sequence of SEQ ID NO: 30. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 30.

In some embodiments, the CAR of the present disclosure comprises an amino acid sequence of SEQ ID NO: 31. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 31.

In some embodiments, the CAR of the present disclosure comprises an amino acid sequence of SEQ ID NO: 32. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 32.

In certain embodiments, the CAR may comprise a signal peptide. In certain embodiments the signal peptide is at the N-terminus of the CAR sequence. In certain embodiments the signal peptide is at the C-terminus of the CAR sequence. In certain embodiments, the signal peptide comprises or consists of the amino acid sequence of SEQ ID No: 11.

### CAR Constructs and Immunoresponsive Cells Expressing CARs

In one aspect, the disclosure provides isolated nucleic acid molecules encoding the CARs described herein. In some embodiments, the nucleic acid molecule comprises a nucleic acid sequence selected from the group consisting of SEQ ID NOS: 90, 89, 87, 88, 33, 34, 35, 36, 37, and 38. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NOS: 90, 89, 87, 88, 33, 34, 35, 36, 37, or 38.

In some embodiments, the nucleic acid molecule comprises a nucleic acid sequence of SEQ ID NO: 90. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 90. In some embodiments, the nucleic acid molecule comprises a nucleic acid sequence of SEQ ID NO: 89. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 89. In some embodiments, the nucleic acid molecule comprises a nucleic acid sequence of SEQ ID NO: 87. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 87. In some embodiments, the nucleic acid molecule comprises a nucleic acid sequence of SEQ ID NO: 88. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 88. In some embodiments, the nucleic acid molecule comprises a nucleic acid sequence of SEQ ID NO: 33. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 33. In some embodiments, the nucleic acid molecule comprises a nucleic acid sequence of SEQ ID NO: 34. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 34. In some embodiments, the nucleic acid molecule comprises a nucleic acid sequence of SEQ ID NO: 35. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 35. In some embodiments, the nucleic acid molecule comprises a nucleic acid sequence of SEQ ID NO: 36. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 36. In some embodiments, the nucleic acid molecule comprises a nucleic acid sequence of SEQ ID NO: 37. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 37. In some embodiments, the nucleic acid molecule comprises a nucleic acid sequence of SEQ ID NO: 38. In some embodiments, the isolated nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 38.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

In some embodiments, the present disclosure provides an expression vector comprising the nucleic acid molecules described (e.g., SEQ ID NOS: 90, 89, 87, 88, 33, 34, 35, 36, 37, or 38). In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NOS: 90, 89, 87, 88, 33, 34, 35, 36, 37, or 38.

In some embodiments, the expression vector comprises a nucleic acid molecule of SEQ ID NO: 90. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 90. In some embodiments, the expression vector comprises a nucleic acid molecule of SEQ ID NO: 89. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 89. In some embodiments, the expression vector comprises a nucleic acid molecule of SEQ ID NO: 87. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 87. In some embodiments, the expression vector comprises a nucleic acid molecule of SEQ ID NO: 88. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 88. In some embodiments, the expression vector comprises a nucleic acid molecule of SEQ ID NO: 33. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 33. In some embodiments, the expression vector comprises a nucleic acid molecule of SEQ ID NO: 34. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 34. In some embodiments, the expression vector comprises a nucleic acid molecule of SEQ ID NO: 35. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 35. In some embodiments, the expression vector comprises a nucleic acid molecule of SEQ ID NO: 36. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 36. In some embodiments, the expression vector comprises a nucleic acid molecule of SEQ ID NO: 37. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 37. In some embodiments, the expression vector comprises a nucleic acid molecule of SEQ ID NO: 38. In some embodiments, the expression vector comprises a nucleic acid molecule comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NO: 38.

The nucleic acid molecules described encode for amino acids with sequences selected from the group consisting of SEQ ID NOS: 86, 85, 83, 84, 27, 28, 29, 30, 31 and 32, or a variant thereof. In some embodiments, the expression vector comprises a nucleic acid molecule encoding the amino acid sequence of SEQ ID NO: 86. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 86. In some embodiments, the expression vector comprises a nucleic acid molecule encoding the amino acid sequence of SEQ ID NO: 85. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 85. In some embodiments, the expression vector comprises a nucleic acid molecule encoding the amino acid sequence of SEQ ID NO: 83. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 83. In some embodiments, the expression vector comprises a nucleic acid molecule encoding the amino acid sequence of SEQ ID NO: 84. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 84. In some embodiments, the expression vector comprises a nucleic acid molecule encoding the amino acid sequence of SEQ ID NO: 27. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 27. In some embodiments, the expression vector comprises a nucleic acid molecule encoding the amino acid sequence of SEQ ID NO: 28. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 28. In some embodiments, the expression vector comprises a nucleic acid molecule encoding the amino acid sequence of SEQ ID NO: 29. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 29. In some embodiments, the expression vector comprises a nucleic acid molecule encoding the amino acid sequence of SEQ ID NO: 30. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 30. In some embodiments, the expression vector comprises a nucleic acid molecule encoding the amino acid sequence of SEQ ID NO: 31. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 31. In some embodiments, the expression vector comprises a nucleic acid molecule encoding the amino acid sequence of SEQ ID NO: 32. In some embodiments, the expression vector comprises a nucleic acid molecule encoding an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 32.

Herein, the structure of nucleic acid molecules is in places defined in the basis of % sequence identity with a recited reference sequence (with a given SEQ ID NO). In this context, % sequence identity regarding nucleic acid molecules refers to the similarity between at least two different nucleic acid sequences. When a position in both of the two compared sequences is occupied by the same base e.g., if a position in each of two DNA molecules is occupied by adenine, then the molecules are identical at that position, The percent of identity between two sequences is a function of the number of matching or homologous positions shared by the two sequences divided by the number of positions compared and multiplied by 100. For example, if 6 of 10 of the positions in two sequences are matched or homologous then the two sequences are 60% identical (or homologous). By way of example, the DNA sequences ATTGCC and TATGGC share 50% identity (or homology). Generally, a comparison is made when two sequences are aligned to give maximum homology. The respective percent identity can be determined by standard alignment algorithms, for example, the Basic Local Alignment Search Tool (BLAST) described by Altshul et al. ((1990) J. MoI. Biol. 215:403-10); the algorithm of Needleman et al. ((1970) J. MoI. Biol. 48:444-53); or the algorithm of Meyers et al. ((1988) Comput. Appl. Biosci. 4:11-17). A set of parameters may be the Blosum 62 scoring matrix with a gap penalty of 12, a gap extend penalty of 4, and a frameshift gap penalty of 5. The percent identity between two nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12, and a gap penalty of 4. The percent identity is usually calculated by comparing sequences of similar length.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In one embodiment, the present disclosure provides a cell expressing the nucleic acid molecule described herein. In one embodiment, the present disclosure provides a cell expressing a CAR of the present disclosure. In one embodiment, the CAR of the present disclosure comprises an amino acid sequence selected from the group consisting of SEQ ID NOS: 86, 85, 83, 84, 27, 28, 29, 30, 31 and 32. In some embodiments, the CAR of the present disclosure comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with any one of SEQ ID NOS: 86, 85, 83, 84, 27, 28, 29, 30, 31 and 32.

In some embodiments, the CAR comprises an amino acid sequence of SEQ ID NO: 86. In some embodiments, the CAR comprises an amino acid sequence of SEQ ID NO: 85. In some embodiments, the CAR comprises an amino acid sequence of SEQ ID NO: 83. In some embodiments, the CAR comprises an amino acid sequence of SEQ ID NO: 84. In some embodiments, the CAR comprises an amino acid sequence of SEQ ID NO: 27. In some embodiments, the CAR comprises an amino acid sequence of SEQ ID NO: 28. In some embodiments, the CAR comprises an amino acid sequence of SEQ ID NO: 29. In some embodiments, the CAR comprises an amino acid sequence of SEQ ID NO: 30. In some embodiments, the CAR comprises an amino acid sequence of SEQ ID NO: 31. In some embodiments, the CAR comprises an amino acid sequence of SEQ ID NO: 32.

In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 86. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 85. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 83. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 84. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 27. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 28. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 29. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 30. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 31. In some embodiments, the CAR comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity with SEQ ID NO: 32.

In one aspect, the present disclosure provides isolated immunoresponsive cells comprising the CARs described herein. In some embodiments, the isolated immunoresponsive cell is transduced with the CAR, for example, the CAR is constitutively expressed on the surface of the immunoresponsive cell. In certain embodiments, the isolated immunoresponsive cell is further transduced with at least one co-stimulatory ligand such that the immunoresponsive cell expresses the at least one co-stimulatory ligand. In certain embodiments, the at least one co-stimulatory ligand is selected from the group consisting of 4-1BBL, CD48, CD70, CD80, CD86, OX40L, TNFRSF14, and combinations thereof. In certain embodiments, the isolated immunoresponsive cell is further transduced with at least one cytokine such that the immunoresponsive cell secretes the at least one cytokine. In certain embodiments, the at least cytokine is selected from the group consisting of IL-2, IL-3, IL-6, IL-7, IL-11, IL-12, IL-15, IL-17, IL-21, and combinations thereof. In some embodiments, the isolated immunoresponsive cell is selected from the group consisting of a T lymphocyte (T cell), a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated.

The inventive nucleic acid sequence encoding a CAR may be introduced into a cell by “transfection”, “transformation”, or “transduction”. “Transfection”, “transformation”, or “transduction”, as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

In one embodiment, the CAR T cells of the disclosure can be generated by introducing a lentiviral vector comprising a desired CAR, for example, a CAR comprising anti-GPRC5D, CD8α hinge and transmembrane domain, and human 4-1BB and CD3-zeta signaling domains, into the cells. The CAR T cells of the invention are able to replicate in vivo resulting in long-term persistence that can lead to sustained tumor control.

Embodiments of the invention further provide host cells comprising any of the recombinant expression vectors described herein. As used herein, the term “host cell” refers to any type of cell that can contain the recombinant expression vector. The host cell can be a eukaryotic cell, e.g., plant, animal, or algae, fungi, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). The host cell may be a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to bone marrow, blood, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD8⁺ T cells (e.g., cytotoxic T cells), CD4⁺ helper T cells, e.g., Th1 and Th2 cells, peripheral blood mononuclear cells (PBMCs), peripheral blood leukocytes (PBLs), tumor infiltrating cells, memory T cells, naïve T cells, and the like. The T cell may be a CD8⁺ T cell or a CD4⁺ T cell.

T cells may also include “NKT cells”, which refer to a specialized population of T cells that express a semi-invariant as T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs” which refer to T cells that suppress an abnormal or excessive immune response and play a role in immune tolerance. Tregs cells are typically transcription factor Foxp3-positive CD4+T cells and can also include transcription factor Foxp3-negative regulatory T cells that are IL-10-producing CD4+T cells.

T-cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-T1 cells (ATCC CRL-1942), RPMI 8402 cells (DSMZ ACC-290), Karpas 45 cells (DSMZ ACC-545), and derivatives thereof.

In another embodiment, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

Also provided are a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, an erythrocyte, a neutrophil, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

### Pharmaceutical Compositions/Administration

In embodiments of the present disclosure, the CAR-expressing cells may be provided in compositions, e.g., suitable pharmaceutical composition(s) comprising the CAR-expressing cells and a pharmaceutically acceptable carrier. In one aspect, the present disclosure provides pharmaceutical compositions comprising an effective amount of a lymphocyte expressing one or more of the CARs described and a pharmaceutically acceptable excipient. Pharmaceutical compositions of the present disclosure may comprise a CAR-expressing cell, e.g., a plurality of CAR-expressing cells, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation

Such compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

In one aspect, the disclosure relates to administering a genetically modified T cell expressing a CAR for the treatment of a subject having cancer or at risk of having cancer using lymphocyte infusion. In at least one embodiment, autologous lymphocyte infusion is used in the treatment. Autologous PBMCs are collected from a subject in need of treatment and T cells are activated and expanded using the methods described herein and known in the art and then infused back into the subject.

In one aspect, the disclosure relates generally to the treatment of a subject at risk of developing cancer. The invention also includes treating a malignancy or an autoimmune disease in which chemotherapy and/or immunotherapy in a subject results in significant immunosuppression, thereby increasing the risk of the subject developing cancer. In one aspect, the present disclosure provides methods of preventing cancer, the methods comprising administering an amount of a lymphocyte expressing one or more of the CARs described to a subject in need thereof.

In one aspect, the present disclosure provides methods of treating a subject having cancer, the methods comprising administering a therapeutically effective amount of a lymphocyte expressing one or more of the CARs described to a subject in need thereof, whereby the lymphocyte induces or modulates killing of cancer cells in the subject.

In another aspect, the present disclosure provides methods of reducing tumor burden in a subject having cancer, the methods comprising administering a therapeutically effective amount of a lymphocyte expressing one or more of the CARs described herein to a subject in need thereof, whereby the lymphocyte induces killing of cancer cells in the subject. In another aspect, the present disclosure provides methods of increasing survival of a subject having cancer, the methods comprising administering a therapeutically effective amount of a lymphocyte expressing one or more of the CARs described to a subject in need thereof, whereby the survival of the subject is lengthened. Generally, the lymphocytes expressing the CAR(s) induce killing of cancer cells in the subject and result in reduction or eradication of the tumors/cancer cells in the subject. A non-limiting list of cancers, inclusive of metastatic lesions, that can be targeted, includes lung cancer, gastric cancer, colon cancer, hepatocellular carcinoma, renal cell carcinoma, bladder urothelial carcinoma, metastatic melanoma, breast cancer, ovarian cancer, cervical cancer, head and neck cancer, pancreatic cancer, glioma, glioblastoma, non-Hodgkin's lymphoma (NHL), acute lymphocytic leukemia (ALL), chronic lymphocytic leukemia (CLL), chronic myelogenous leukemia (CML), smoldering multiple myeloma (SMM), multiple myeloma (MM), acute myeloid leukemia (AML), and combinations thereof. In one embodiment, the cancer being treated in a subject is multiple myeloma.

In one aspect, the methods described herein are applicable to treatment of non-cancerous conditions that are at risk of developing into a cancerous condition, such as, e.g., monoclonal gammopathy of undetermined significance (MGUS), which is at risk of developing into a blood cancer, such as MM or lymphoma, and myelodysplastic syndrome, which is at risk of developing into a blood cancer, such as leukemia.

In one aspect, methods of treating a subject having cancer are provided that comprise administering a therapeutically effective amount of a lymphocyte expressing a CAR, the CAR having an extracellular antigen-binding domain that binds the GPRC5D antigen, to a subject in need thereof, whereby the lymphocyte induces killing of cancer cells in the subject. In some embodiments, the at least one of the CARs comprises an amino acid sequence selected from the group consisting of SEQ ID NOS: 86, 85, 83, 84, 27, 28, 29, 30, 31 and 32.

In one aspect, a method of targeted killing of a cancer cell is disclosed, the method comprising contacting the cancer cell with a lymphocyte expressing one or more of the CARs described, whereby the lymphocyte induces killing of the cancer cell. A non-limiting list of cancer cells, inclusive of metastatic cancer cells, that can be targeted include a lung cancer cell, a gastric cancer cell, a colon cancer cell, a hepatocellular carcinoma cell, a renal cell carcinoma cell, a bladder urothelial carcinoma cell, a metastatic melanoma cell, a breast cancer cell, an ovarian cancer cell, a cervical cancer cell, a head and neck cancer cell, a pancreatic cancer cell, a glioma cell, a glioblastoma cell, and a non-Hodgkin's lymphoma (NHL) cell, an acute lymphocytic leukemia (ALL) cell, a chronic lymphocytic leukemia (CLL) cell, a chronic myelogenous leukemia (CML) cell, a smoldering multiple myeloma (SMM) cell, a multiple myeloma (MM) cell, an acute myeloid leukemia (AML) cell, and combinations thereof. In one embodiment, the cancer cell is a multiple myeloma cell.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

The terms “treat” or “treatment” refer to therapeutic treatment wherein the object is to slow down (lessen) an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to have the physiological change or disease.

A “therapeutically effective amount” or “effective amount”, used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body.

As used herein, the term “subject” refers to an animal. The terms “subject” and “patient” may be used interchangeably herein in reference to a subject. As such, a “subject” includes a human that is being treated for a disease, or prevention of a disease, as a patient.

The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). In one embodiment, the mammal is a human.

When a therapeutically effective amount is indicated, the precise amount of the compositions of the present disclosure to be administered can be determined by a physician with consideration of individual differences in age, weight, tumor size, extent of infection or metastasis, and condition of the subject. It can generally be stated that a pharmaceutical composition comprising the T cells described herein may be administered at a dosage of about 10⁴ to about 10¹⁰ cells/kg body weight, in some instances about 10⁵ to about 10⁶ cells/kg body weight, including all integer values within those ranges. In some embodiments, a pharmaceutical composition comprising the T cells described herein may be administered at a dosage of about 10⁶ cells/kg body weight. T cell compositions may also be administered multiple times at these dosages. The cells can be administered by using infusion techniques that are commonly known in immunotherapy (see, e.g., Rosenberg et al., New Eng. J. of Med. 319:1676, 1988).

Delivery systems useful in the context of embodiments of the invention may include time-released, delayed release, and sustained release delivery systems such that the delivery of the T cell compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

In certain aspects, it may be desirable to administer activated T cells to a subject and then subsequently redraw blood (or have an apheresis performed), activate the T cells according to the present disclosure, and reinfuse the subject with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the CAR-T cells and compositions may be carried out in any manner, e.g., by parenteral or nonparenteral administration, including by aerosol inhalation, injection, infusions, ingestion, transfusion, implantation or transplantation. For example, the CAR-T cells and compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection. The compositions of T cells may be injected, for instance, directly into a tumor, lymph node, tissue, organ, or site of infection.

Administration can be autologous or non-autologous. For example, immunoresponsive cells expressing a G-protein coupled receptor (e.g., GPRC5D)-specific CAR can be obtained from one subject, and administered to the same subject or a different, compatible subject. Peripheral blood derived T cells of the present disclosure, or expanded T cells (e.g., in vivo, ex vivo or in vitro derived) can be administered via, e.g., intravenous injection, localized injection, systemic injection, catheter administration, or parenteral administration.

In particular embodiments, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more CAR constructs of the present disclosure may be introduced, thereby creating a CAR-T cell. Subjects in need thereof may subsequently undergo standard treatment with high dose chemotherapy followed by peripheral blood stem cell transplantation. In certain aspects, following or concurrent with the transplant, subjects receive an infusion of the expanded CAR-T cells. In one aspect, expanded cells are administered before or following surgery.

The dosage administered to a patient having a malignancy is sufficient to alleviate or at least partially arrest the disease being treated (“therapeutically effective amount”). The dosage of the above treatments to be administered to a subject will vary with the precise nature of the condition being treated and the recipient of the treatment. The scaling of dosages for human administration can be performed according to practices generally accepted in the art.

The CART T cells of the invention can undergo in vivo T cell expansion and can establish GPRC5D-specific memory cells that persist at high levels for an extended amount of time in blood and bone marrow. In some instances, the CAR T cells of the invention infused into a subject can eliminate cancer cells, e.g., leukemia cells, in vivo in subjects with advanced chemotherapy-resistant cancer.

In one embodiment, a CAR of the present disclosure is introduced into T cells, e.g., using in vitro transcription, and the subject (e.g., human) receives an initial administration of CAR-T cells of the disclosure, and one or more subsequent administrations of the CAR-T cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-T cells are administered to the subject (e.g., human) per week, e.g., 2, 3, or 4 administrations of the CAR-T cells are administered per week. In one embodiment, the subject receives more than one administration of the CAR-T cells per week (e.g., 2, 3 or 4 administrations per week) (also referred to herein as a cycle), followed by a week of no CAR-T cell administrations, and then one or more additional administration of the CAR-T cells (e.g., more than one administration of the CAR-T cells per week) is administered to the subject. In another embodiment, the subject receives more than one cycle of CAR-T cells, and the time between each cycle is less than 10, 9, 8, 7, 6, 5, 4, or 3 days. In one embodiment, the CAR-T cells are administered every other day for 3 administrations per week. In one embodiment, the CAR-T cells are administered for at least two, three, four, five, six, seven, eight or more weeks.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The CAR-T cells may be administered in the methods of the invention by maintenance therapy, such as, e.g., once a week for a period of 6 months or more.

In one embodiment, CAR-T cells are generated using lentiviral viral vectors, such as lentivirus. CAR-T cells generated with such viral vectors will generally have stable CAR expression.

In one embodiment, CAR-T cells transiently express CAR vectors for 4, 5, 6, 7, 8, 9, 10, 11, 12, 13, 14, 15 days after transduction. Transient expression of CARs can be affected by RNA CAR vector delivery. In one embodiment, the CAR RNA is transduced into the T cell by electroporation.

If a patient is at high risk of generating an anti-CAR antibody response during the course of transient CAR therapy (such as those generated by RNA transductions), CAR-T infusion breaks should not last more than ten to fourteen days.

A CAR-expressing cell described herein may be used in combination with other known agents and therapies. Administered “in combination”, as used herein, means that two (or more) different treatments are delivered to the subject during the course of the subject's treatment e.g., the two or more treatments are delivered after the subject has been diagnosed with the cancer and before the cancer has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one embodiment, other therapeutic agents such as factors may be administered before, after, or at the same time (simultaneous with) as the CAR-T cells, including, but not limited to, interleukins, e.g. IL-2, IL-3, IL 6, IL-7, IL-11, IL-12, IL-15, IL-21, as well as the other interleukins, colony stimulating factors, such as G-, M- and GM-CSF, and interferons, e.g., y-interferon.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In further embodiments, a CAR-expressing cell described herein may be used in a treatment regimen in combination with surgery, radiation, chemotherapy, immunosuppressive agents, such as methotrexate, cyclosporin, azathioprine, mycophenolate, and FK506, antibodies, or other immunoablative agents such as anti-CD3 antibodies or other antibody therapies, cytoxin, 5 fludarabine, cyclosporin, FK506, rapamycin, mycophenolic acid, steroids, FR901228, cytokines, and irradiation.

In one embodiment, a CAR-expressing cell described herein can be used in combination with a chemotherapeutic agent. Example chemotherapeutic agents include, but are not limited to, an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

A non-exhaustive list of chemotherapeutic agents considered for use in combination therapies include anastrozole (Arimidex®), bicalutamide (Casodex®), bleomycin sulfate (Blenoxane®), busulfan (Myleran®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), tamoxifen citrate (Nolvadex®), teniposide (Vumon®), 6-thioguanine, thiotepa, tirapazamine (Tirazone®), topotecan 5 hydrochloride for injection (Hycamptin®), vinblastine (Velban®), vincristine (Oncovin®), and vinorelbine (Navelbine®).

Example alkylating agents include, without limitation, nitrogen mustards, ethylenimine derivatives, alkyl sulfonates, nitrosoureas and triazenes): uracil mustard (Aminouracil Mustard®, Chlorethaminacil®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), Demethyldopan®, Desmethyldopan®, triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional example alkylating agents include, without limitation, Oxaliplatin (Eloxatin®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Temozolomide (Temodar® and Temodal®); Dactinomycin (also known as actinomycin-D, Cosmegen®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of immunomodulators useful herein include, but are not limited to, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

In one embodiment, the subject can be administered an agent which enhances the activity of a CAR-expressing cell. For example, in one embodiment, the agent can be an agent which inhibits an inhibitory molecule. Inhibitory molecules, e.g., Programmed Death 1 (PD1), can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta.

A description of example embodiments follows.

Embodiment 1. A chimeric antigen receptor (CAR), comprising an extracellular antigen-binding domain, a transmembrane domain and an intracellular signaling domain, wherein the extracellular antigen-binding domain comprises:
a heavy chain complementarity determining region 1 (CDR1) having the amino acid sequence of SEQ ID NO: 66, a heavy chain CDR2 having the amino acid sequence of SEQ ID NO: 67, and a heavy chain CDR3 having the amino acid sequence of SEQ ID NO: 68;
a heavy chain complementarity determining region 1 (CDR1) having the amino acid sequence of SEQ ID NO: 58, a heavy chain CDR2 having the amino acid sequence of SEQ ID NO: 59, and a heavy chain CDR3 having the amino acid sequence of SEQ ID NO: 60;
a heavy chain complementarity determining region 1 (CDR1) having the amino acid sequence of SEQ ID NO: 39, a heavy chain CDR2 having the amino acid sequence of SEQ ID NO: 40, and a heavy chain CDR3 having the amino acid sequence of SEQ ID NO: 41;
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44; or
a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47;
wherein the extracellular antigen-binding domain binds the anti-G protein receptor family C group 5 member D (GPRC5D) antigen.

Embodiment 2. The CAR of Embodiment 1, wherein
the extracellular antigen-binding domain comprising the heavy chain CDR1 having the amino acid sequence of SEQ ID NO: 66, the heavy chain CDR2 having the amino acid sequence of SEQ ID NO: 67, and the heavy chain CDR3 having the amino acid sequence of SEQ ID NO: 68, and further comprises a light chain CDR1 having the amino acid sequence of SEQ ID NO: 69, a light chain CDR2 having the amino acid sequence of SEQ ID NO: 70, and a light chain CDR3 having the amino acid sequence of SEQ ID NO: 71;
the extracellular antigen-binding domain comprising the heavy chain CDR1 having the amino acid sequence of SEQ ID NO: 58, the heavy chain CDR2 having the amino acid sequence of SEQ ID NO: 59, and the heavy chain CDR3 having the amino acid sequence of SEQ ID NO: 60, and further comprises a light chain CDR1 having the amino acid sequence of SEQ ID NO: 61, a light chain CDR2 having the amino acid sequence of SEQ ID NO: 62, and a light chain CDR3 having the amino acid sequence of SEQ ID NO: 63;
the extracellular antigen-binding domain comprising the heavy chain CDR1 having the amino acid sequence of SEQ ID NO: 39, the heavy chain CDR2 having the amino acid sequence of SEQ ID NO: 40, and the heavy chain CDR3 having the amino acid sequence of SEQ ID NO: 41, and further comprises a light chain CDR1 having the amino acid sequence of SEQ ID NO: 48, a light chain CDR2 having the amino acid sequence of SEQ ID NO: 49, and a light chain CDR3 having the amino acid sequence of SEQ ID NO: 50;
the extracellular antigen-binding domain comprising the heavy chain CDR1 having the amino acid sequence of SEQ ID NO: 42, the heavy chain CDR2 having the amino acid sequence of SEQ ID NO: 43, and the heavy chain CDR3 having the amino acid sequence of SEQ ID NO: 44, and further comprises a light chain CDR1 having the amino acid sequence of SEQ ID NO: 51, a light chain CDR2 having the amino acid sequence of SEQ ID NO: 52, and a light chain CDR3 having the amino acid sequence of SEQ ID NO: 53; or
the extracellular antigen-binding domain comprising the heavy chain CDR1 having the amino acid sequence of SEQ ID NO: 45, the heavy chain CDR2 having the amino acid sequence of SEQ ID NO: 46, and the heavy chain CDR3 having the amino acid sequence of SEQ ID NO: 47, and further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56.

Embodiment 3. The CAR of Embodiment 1 or 2, wherein the extracellular antigen-binding domain comprises a light chain variable region (LCVR) comprising an amino acid sequence selected from the group consisting of SEQ ID NOS: 73, 65, 1, 3 and 5, or a heavy chain variable region (HCVR) comprising an amino acid sequence selected from the group consisting of SEQ ID NOS: 72, 64, 2, 4, and 6, or a combination of a LCVR comprising an amino acid sequence selected from the group consisting of SEQ ID NOS: 73, 65, 1, 3 and 5, and a HCVR comprising an amino acid sequence selected from the group consisting of SEQ ID NOS: 72, 64, 2, 4, and 6.

Embodiment 4. The CAR of Embodiments 1-3, wherein the extracellular antigen-binding domain comprises:
a light chain variable region comprising an amino acid sequence of SEQ ID NO: 73 and a heavy chain variable region comprising an amino acid sequence of SEQ ID NO: 72;
a light chain variable region comprising an amino acid sequence of SEQ ID NO: 65 and a heavy chain variable region comprising an amino acid sequence of SEQ ID NO: 64;
a light chain variable region comprising an amino acid sequence of SEQ ID NO: 1 and a heavy chain variable region comprising an amino acid sequence of SEQ ID NO: 2;
a light chain variable region comprising an amino acid sequence of SEQ ID NO: 3 and a heavy chain variable region comprising an amino acid sequence of SEQ ID NO: 4; or
a light chain variable region comprising an amino acid sequence of SEQ ID NO: 5 and a heavy chain variable region comprising an amino acid sequence of SEQ ID NO: 6.

Embodiment 5. The CAR of any of Embodiments 1-4, wherein the extracellular antigen-binding domain comprises a single-chain variable fragment (scFv).

Embodiment 6. The CAR of Embodiment 5, wherein the scFv comprises a linker polypeptide between the light chain variable region and the heavy chain variable region.

Embodiment 7. The CAR of Embodiment 6, wherein the linker polypeptide comprises an amino acid sequence of SEQ ID NO: 7.

Embodiment 8. The CAR of any of Embodiments 5-7, wherein the scFv comprises an amino acid sequence selected from the group consisting of SEQ ID NOS: 78, 77, 75, 76, 8, 9, 10, 24, 25, and 26.

Embodiment 9. The CAR of any of Embodiments 1-8, wherein the extracellular antigen-binding domain comprises a signal polypeptide.

Embodiment 10. The CAR of Embodiment 9, wherein the signal polypeptide comprises an amino acid sequence of SEQ ID NO: 11.

Embodiment 11. The CAR of any of Embodiments 1-10, wherein the intracellular signaling domain comprises a polypeptide component selected from the group consisting of a TNF receptor superfamily member 9 (CD137) component, a T-cell surface glycoprotein CD3 zeta chain (CD3z) component, a cluster of differentiation (CD27) component, a cluster of differentiation superfamily member component, and a combination thereof.

Embodiment 12. The CAR of Embodiment 11, wherein the CD137 component comprises an amino acid sequence of SEQ ID NO: 12.

Embodiment 13. The CAR of Embodiment 11, wherein the CD3z component comprises an amino acid sequence of SEQ ID NO: 13.

Embodiment 14. The CAR of Embodiment 11, wherein the intracellular signaling domain comprises an amino acid sequence of SEQ ID NO: 14.

Embodiment 15. The CAR of any of Embodiments 1-14, wherein the transmembrane domain comprises a CD8α transmembrane region (CD8a-TM) polypeptide.

Embodiment 16. The CAR of Embodiment 15, wherein the CD8a-TM polypeptide comprises an amino acid sequence of SEQ ID NO: 15.

Embodiment 17. The CAR of any of Embodiments 1-16, further comprising a hinge region linking the transmembrane domain to the extracellular antigen-binding domain.

Embodiment 18. The CAR of Embodiment 17, wherein the hinge region is a CD8a-hinge region.

Embodiment 19. The CAR of Embodiment 18, wherein the CD8a-hinge region comprises an amino acid sequence of SEQ ID NO: 16.

Embodiment 20. The CAR of any of Embodiments 1-19, wherein the extracellular antigen-binding domain comprises an amino acid sequence selected from the group consisting of SEQ ID NOS: 82, 81, 80, 79, 17, 18, 19, 20, 21, and 22.

Embodiment 21. The CAR of any of Embodiments 1-20, wherein the CAR comprises an amino acid sequence selected from the group consisting of SEQ ID NOS: 86, 85, 83, 84, 27, 28, 29, 30, 31 and 32.

Embodiment 22. An isolated lymphocyte expressing the CAR of any of Embodiments 1-21.

Embodiment 23. The isolated lymphocyte of Embodiment 22, wherein the lymphocyte is a T lymphocyte.

Embodiment 24. An isolated nucleic acid molecule encoding the CAR of any of Embodiments 1-21.

Embodiment 25. The isolated nucleic acid molecule of Embodiment 24, wherein the nucleic acid molecule comprises a nucleic acid sequence selected from the group consisting of SEQ ID NOS: 90, 89, 87, 88, 33, 34, 35, 36, 37, and 38.

Embodiment 26. The isolated nucleic acid molecule of Embodiment 24, wherein the nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NOS: 90, 89, 87, 88, 33, 34, 35, 36, 37, and 38.

Embodiment 27. A vector comprising the nucleic acid molecule of any of Embodiments 24-26.

Embodiment 28. A cell expressing the nucleic acid molecule of any of Embodiments 24-26.

Embodiment 29. A pharmaceutical composition, comprising an effective amount of the lymphocyte of any of Embodiments 22-23.

Embodiment 30. A pharmaceutical composition, comprising an effective amount of the lymphocyte of any of Embodiments 22-23 and a pharmaceutically acceptable excipient.

Embodiment 31. The CAR of any of Embodiments 1-21 or the pharmaceutical composition of Embodiments 29 or 30 for use in therapy.

Embodiment 32. The CAR of any of Embodiments 1-21 or the pharmaceutical composition of Embodiments 29 or 30 for use in a method of treating a subject having cancer.

Embodiment 33. A method of treating a subject having cancer, the method comprising:
administering a therapeutically effective amount of the lymphocyte of any of Embodiments 22-23 to a subject in need thereof, whereby the lymphocyte induces killing of cancer cells in the subject.

Embodiment 34. The method of Embodiment 33, or the CAR or the pharmaceutical composition for use in a method of Embodiment 32, wherein the cancer is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and a non-Hodgkin's lymphoma (NHL), an acute lymphocytic leukemia (ALL), a chronic lymphocytic leukemia (CLL), a chronic myelogenous leukemia (CML), a smoldering multiple myeloma (SMM), a multiple myeloma (MM), an acute myeloid leukemia (AML), and combinations thereof.

Embodiment 35. The method of Embodiment 33 or 34, or the CAR or the pharmaceutical composition for use in a method of Embodiment 32 or 34 wherein the cancer is multiple myeloma.

Embodiment 36. A method of targeted killing of a cancer cell, the method comprising:
contacting the cancer cell with the lymphocyte of any of Embodiments 22-23, whereby the lymphocyte induces killing of the cancer cell.

Embodiment 37. The method of Embodiment 36, wherein the cancer cell is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a smoldering multiple myeloma (SMM), a multiple myeloma (MM), an acute myeloid leukemia (AML), and other liquid tumors, and combinations thereof

Embodiment 38 The method of Embodiment 36 or 37, wherein the cancer cell is a multiple myeloma cell.

Embodiment 39. A method of detecting the presence of cancer in a subject, comprising:
(a) contacting a cell sample obtained from the subject with the CAR of claim 1, thereby forming a CAR-cell complex, and
(b) detecting the complex, wherein detection of the complex is indicative of the presence of cancer in the subject. Preferably the embodiment is performed in vitro.

### EXAMPLES

The following examples are provided to further describe some of the embodiments disclosed herein. The examples are intended to illustrate, not to limit, the disclosed embodiments.

### Example 1—Expression of α-GPRC5D scFv CARs

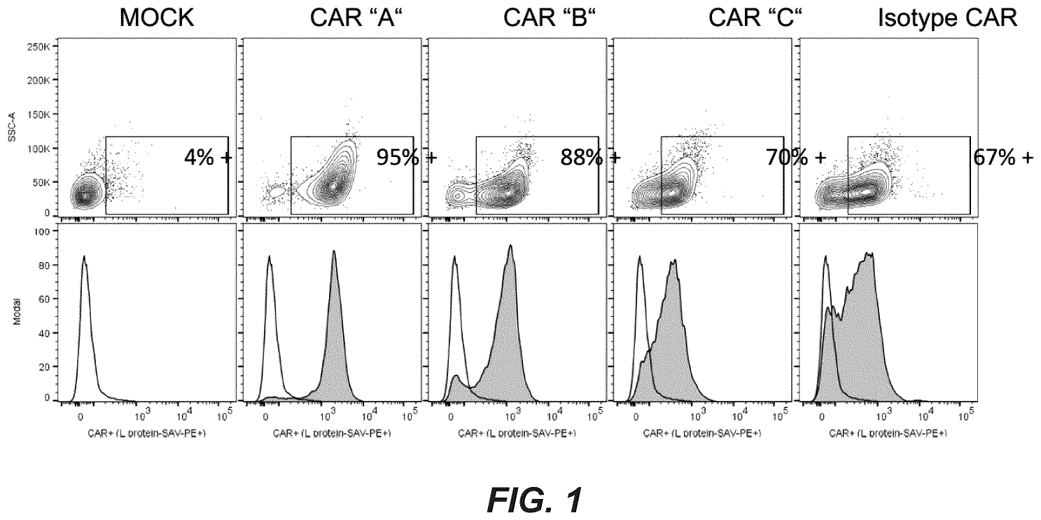

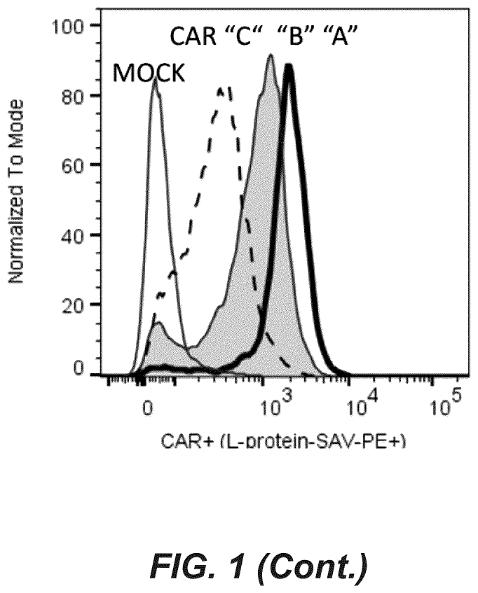

Primary human pan T cells were electroporated with no mRNA (mock) or 10 μg of mRNA expressing either an α-GPRC5D scFv CAR or isotype control CAR. 24 hours post-electroporation CAR surface expression was measured by flow cytometry following stain with biotinylated L-protein and streptavidin-conjugated PE. (FIG. 1). Percent CAR expression was determined by staining with Protein L. Expression was determined to be 95, 88, 70, and 67% positive for CAR A, CAR B, CAR C, and Isotype CAR, respectively. Mock T cells were electroporated only, but no mRNA was added to the cells. The Mock T cells did not express any CAR structure, as expected. These data indicated that the T cells expressed the CAR structure at a high degree. Open histogram is mock, filled grey histogram is CAR-T population.

The amino acid sequences for the components of the GPRC5D-targeting CAR constructs were as shown in Table 2.

TABLE 2

| Domain | Sequence |
|---|---|
| signal | MAWVWTLLFLMAAAQSIQA |
| sequence | (SEQ ID NO: 11) |
| extra- | EIVLTQSPATLSLSPGERATLSCRASQSVSSYLAWYQQKPG |
| cellular | QAPRLLIYDASNRATGIPARFSGSGSGTDFTLTISSLEPED |
| GPRC5D- | FAVYYCQQRSNWPLTFGQGTKVEIKGTEGKSSGSGSESKST |
| specific | EVQLVQSGAEVKKPGESLKISCKGSGYSFTSYFIGWVRQM |
| scFv | PGKGLEWMGIIYPGKSDTRYSPSFQGQVTISADKSISTAYL |
| | QWSSLKASDTAMYYCARVYSFGGRHKALFDYWGQGTLV |
| | TVSS |
| | (SEQ ID NO: 8) |
| | DIQMTQSPSSLSASVGDRVTITCRASQSISSYLNWYQQKPG |
| | KAPKLLIYAASSLQSGVPSRFSGSGSGTDFTLTISSLQPED |
| | FATYYCQQSYSTPLTFGQGTKVEIKGTEGKSSGSGSESKST |
| | QVQLVQSGAEVKKPGSSVKVSCKASGGTFSSYAISWVRQ |
| | APGQGLEWMGGIIPIFGTANYAQKFQGRVTITADESTSTAY |
| | MELSSLRSEDTAVYYCARESRWRGYKLDYWGQGTLVTV |
| | SS |
| | (SEQ ID NO: 9) |
| | DIQMTQSPSSLSASVGDRVTITCKASQNVATHVGWYQQKPG |
| | KAPKRLIYSASYRYSGVPSRFSGSGSGTEFTLTISNLQPED |
| | FATYYCQQYNRYPYTFGQGTKLEIKGTEGKSSGSGSESKS |
| | TQVQLVQSGAEVKKPGASVKVSCKASGYSFTGYTMNWV |
| | RQAPGQGLEWMGLINPYNSDTNYAQKLQGRVTMTTDTS |
| | TSTAYMELRSLRSDDTAVYYCARVALRVALDYWGQGTLV |
| | TVSS |
| | (SEQ ID NO: 10) |
| | EVQLVQSGAEVKKPGESLKISCKGSGYSFTSYFIGWVRQM |
| | PGKGLEWMGIIYPGKSDTRYSPSFQGQVTISADKSISTAYL |
| | QWSSLKASDTAMYYCARVYSFGGRHKALFDYWGQGTLVT |
| | VSSGTEGKSSGSGSESKSTEIVLTQSPATLSLSPGERATLS |
| | CRASQSVSSYLAWYQQKPGQAPRLLIYDASNRATGIPARF |
| | SGSGSGTDFTLTISSLEPEDFAVYYCQQRSNWPLTFGQGTK |
| | VEIK |
| | (SEQ ID NO: 24) |
| | QVQLVQSGAEVKKPGSSVKVSCKASGGTFSSYAISWVRQ |
| | APGQGLEWMGGIIPIFGTANYAQKFQGRVTITADESTSTAY |
| | MELSSLRSEDTAVYYCARESRWRGYKLDYWGQGTLVTV |
| | SSGTEGKSSGSGSESKSTDIQMTQSPSSLSASVGDRVTITC |
| | RASQSISSYLNWYQQKPGKAPKLLIYAASSLQSGVPSRFS |
| | GSGSGTDFTLTISSLQPEDFATYYCQQSYSTPLTFGQGTKV |
| | EIK |
| | (SEQ ID NO: 25) |
| | QVQLVQSGAEVKKPGASVKVSCKASGYSFTGYTMNWVR |
| | QAPGQGLEWMGLINPYNSDTNYAQKLQGRVTMTTDTSTS |
| | TAYMELRSLRSDDTAVYYCARVALRVALDYWGQGTLVTV |
| | SSGTEGKSSGSGSESKSTDIQMTQSPSSLSASVGDRVTITC |
| | KASQNVATHVGWYQQKPGKAPKRLIYSASYRYSGVPSRF |
| | SGSGSGTEFTLTISNLQPEDFATYYCQQYNRYPYTFGQGTK |
| | LEIK |
| | (SEQ ID NO: 26) |
| | QLQLQESGPGLVKPSETLSLTCTVSGGSLSSSSYWWGWTR |
| | QPPGRGLEWIGTMYYSGNIYYNPSLQSRATISVDTSKNQF |
| | SLKLSSVTAADTAVYYCARHVGYSYGRRFWYFDLWGRG |
| | TLVTVSSGGSEGKSSGSGSESKSTGGSEIVLTQSPATLSLS |
| | PGERATLSCRASQSVSSYLAWYQQKPGQAPRLLIYDASNR |
| | ATGIPARFSGSGSGTDFTLTISSLEPEDFAVYYCQQRSNWP |
| | PTFGQGTKVEIK |
| | (SEQ ID NO: 75) |
| | EIVLTQSPATLSLSPGERATLSCRASQSVSSYLAWYQQKPG |
| | QAPRLLIYDASNRATGIPARFSGSGSGTDFTLTISSLEPED |
| | FAVYYCQQRSNWPPTFGQGTKVEIKGGSEGKSSGSGSESKS |
| | TGGSQLQLQESGPGLVKPSETLSLTCTVSGGSLSSSSYWW |
| | GWTRQPPGRGLEWIGTMYYSGNIYYNPSLQSRATISVDTS |
| | KNQFSLKLSSVTAADTAVYYCARHVGYSYGRRFWYFDL |
| | WGRGTLVTVSS |
| | (SEQ ID NO: 76) |
| | QVTLKESGPVLVKPTETLTLTCTVSGFSLTNIRMSVSWIRQ |
| | PPGKALEWLAHIFSNDEKSYSSSLKSRLTISRDTSKSQVVL |
| | TLTNVDPVDTATYYCARMRLPYGMDVWGQGTTVTVSSG |
| | GSEGKSSGSGSESKSTGGSDIVMTQTPLSSPVTLGQPASIS |
| | CRSSQSLVHSDGNTYLSWLQQRPGQPPRLLIYKISNRFFG |
| | VPDRFSGSGAGTDFTLKISRVEAEDVGVYYCMQATQFPH |
| | TFGQGTKLEIK |
| | (SEQ ID NO: 77) |
| | DIVMTQTPLSSPVTLGQPASISCRSSQSLVHSDGNTYLSWL |
| | QQRPGQPPRLLIYKISNRFFGVPDRFSGSGAGTDFTLKISR |
| | VEAEDVGVYYCMQATQFPHTFGQGTKLEIKGGSEGKSSG |
| | SGSESKSTGGSQVTLKESGPVLVKPTETLTLTCTVSGFSLT |
| | NIRMSVSWIRQPPGKALEWLAHIFSNDEKSYSSSLKSRLTI |
| | SRDTSKSQVVLTLTNVDPVDTATYYCARMRLPYGMDVW |
| | GQGTTVTVSS |
| | (SEQ ID NO: 78) |
| human | TSTPAPRPPTPAPTIASQPLSLRPEACRPAAGGAVHTRGLD |
| CD8 | FACD |
| hinge | (SEQ ID NO: 16) |
| sequence | |
| human | IYIWAPLAGTCGVLLLSLVITLYC |
| CD8 | (SEQ ID NO: 15) |
| TM | |
| domain | |
| sequence | |
| human | KRGRKKLLYIFKQPFMRPVQTTQEEDGCSCRFPEEEEGGC |
| CD137 | EL |
| intra- | (SEQ ID NO: 12) |
| cellular | |
| sequence | |
| human | RVKFSRSADAPAYKQGQNQLYNELNLGRREEYDVLDKRR |
| CD3 | GRDPEMGGKPRRKNPQEGLYNELQKDKMAEAYSEIGMK |
| zeta | GERRRGKGHDGLYQGLSTATKDTYDALHMQALPPR |
| intra- | (SEQ ID NO: 13) |
| cellular | |
| domain | |

### Example 2—Induction of Activation Markers in Antigen-Stimulated CAR-T Cells

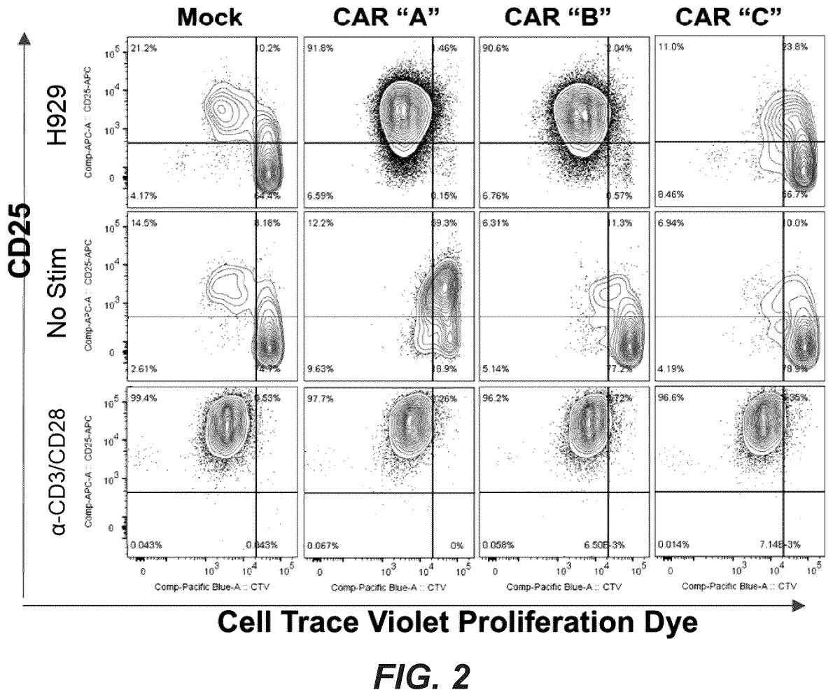

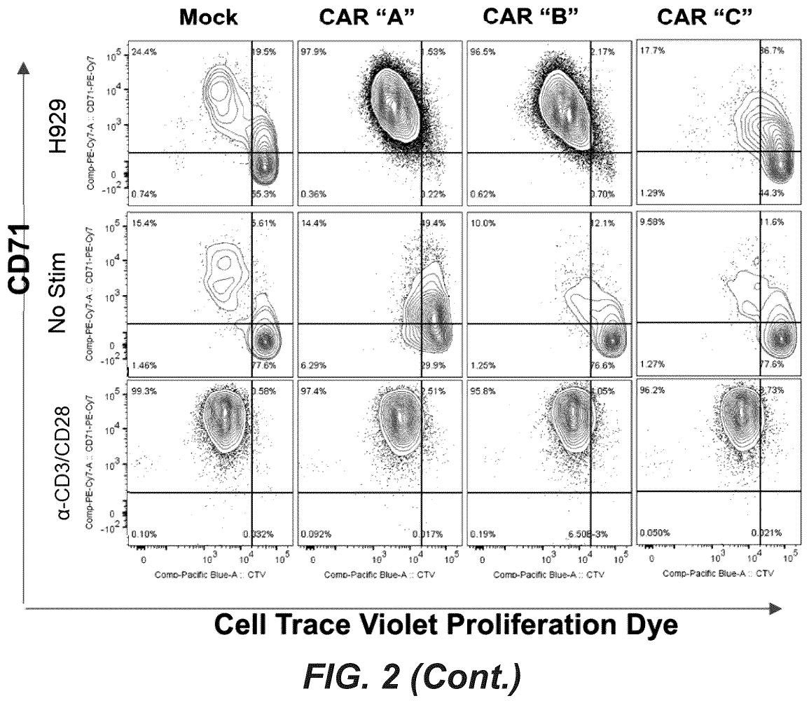

Twenty-four hours after transient transfection, primary pan T cells were labeled with Cell Trace Violet (CTV) fluorescent proliferation dye and then co-cultured with multiple myeloma cell line, H929. Four days post co-culture, cells were pre-gated on the CD8+CD4− population and surface expression of activation markers CD25 and CD71 on CAR-T were compared to T cells cultured alone or in the presence of α-CD3/CD28 beads. (FIG. 2). The same CAR T cells from FIG. 1 were analyzed for a T cell activation response to GPRC5D antigen expressing-H929 cells. Four days after electroporation, CAR A and CAR B showed an increase in T cell activation response, shown by an increase in CD25 and CD71 expression in response to culture with H929 cells. In comparison, the Mock and CAR C-expressing cells demonstrated diminished T cell activation in response to H929 cells. As a negative control, the same cells were grown in the absence of any GPRC5D or T cell antigen and showed minimal increases in CD25 or CD71. As a positive control, Mock, CAR A, CAR B, and CAR C cells were co-cultured with CD3/CD28 beads. Each of the four cell populations showed similar increases in CD25 and CD71 expression. In totality, these data show that CAR A and CAR B cells demonstrated the greatest T cell activation response to GPRC5D-expressing H929 cells.

### Example 3—Cytokine Profiling of Antigen-Stimulated CAR-T Cells

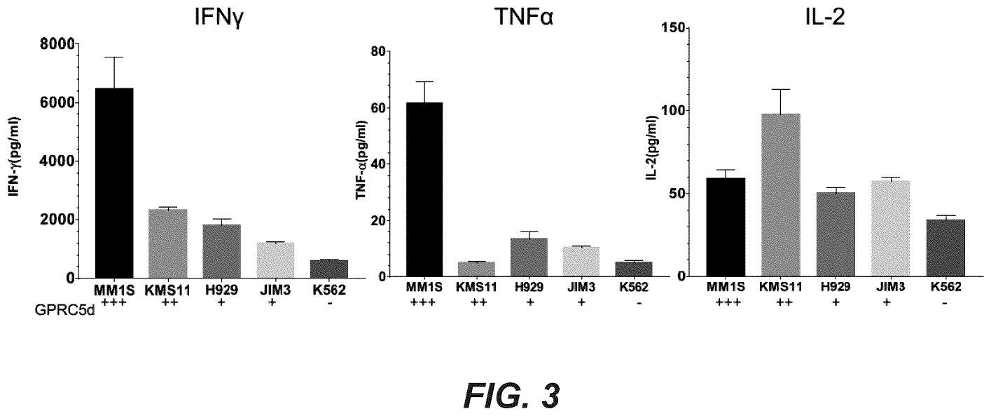

Primary pan T cells transiently transfected 24 hours prior with CAR-expressing mRNA were co-cultured at a 1:1 ratio with various myeloma cell lines, expressing target antigen, GPRC5D, at high (+++), medium (++), low (+) or negative (−) levels. Sixteen hours post co-culture, supernatants were harvested and cytokine profile analysis by Meso Scale Discovery (MSD) of supernatants from co-cultures was performed. (FIG. 3). Supernatants from GPRC5D CAR-T cells showed an increase in pro-inflammatory cytokines INF-γ, TNF-α, or IL-2. Increases in each of the pro-inflammatory cytokines was respective of GPRC5D expression the cell lines co-cultured with the GPRC5D CAR-T cells. Co-culture with MM.1 S resulted in the highest INF-γ and TNF-α expression, whereas co-culture with KMS11 cells resulted in highest IL-2 levels. The K562 cell line was used as negative control, demonstrating basal background cytokines levels with a non-GPRC5D-expressing cell line.

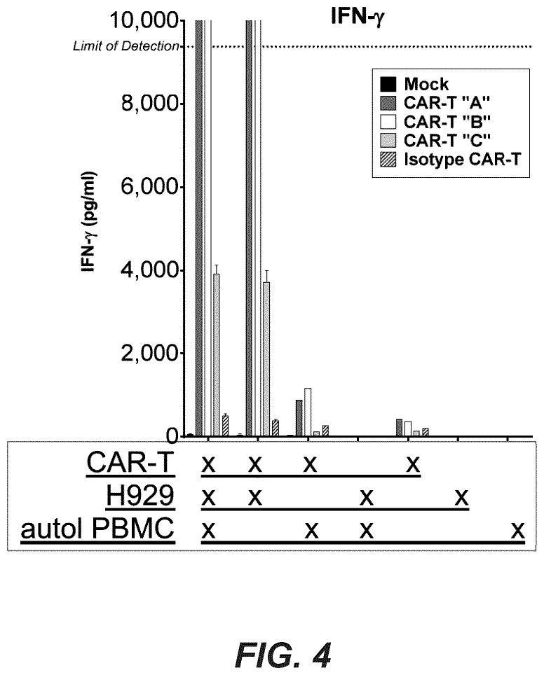

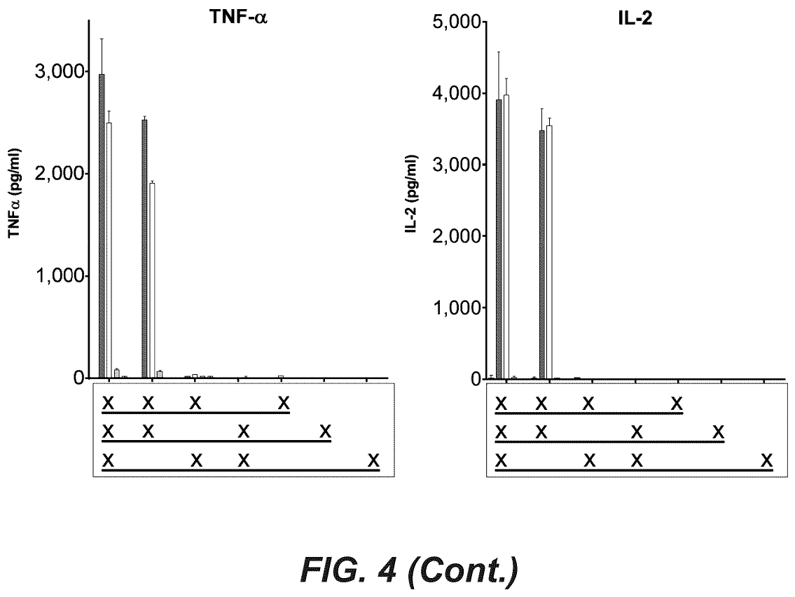

Twenty-four hours after transient transfection with CAR-expressing mRNA, primary pan T cells were co-cultured (at a 1:1:1 ratio) with autologous peripheral blood mononuclear cells (PBMC) and the multiple myeloma cell line, H929. Sixteen hours post co-culture, supernatants were harvested. Cytokine profile analysis by MSD of supernatants from co-cultures containing all three populations (CAR-T, H929, autologous PBMC) were compared to control co-cultures lacking CAR-T cells, PBMC or H929, to each population of cells cultured in isolation. (FIG. 4). Similar to FIG. 3, Mock, CAR-T A, CAR-T B, CAR-T C, or Isotype CAR-T cells were co-cultured with H929 and/or autologous PBMCs. Increases in TNF-α and IL-2 were observed with CAR-T A and CAR-T B cells when H929 cells were co-cultured with PBMCs. When the CAR-T C cells were co-cultured with H929 and PBMC, limited-to-no detectable levels of INF-γ release were observed. Overall, CAR-T C cells demonstrated a diminished cytokine release, consistent with reduced T cell activation markers in FIG. 2. CAR-T, H929, and PBMC cells were cultured alone as negative controls and showed background cytokine levels.

### Example 4—Targeted Cytotoxicity of GPRC5D-Expressing Tumor Cell Lines: High/Low/Non-Expressing GPRC5D Target Cell Lines

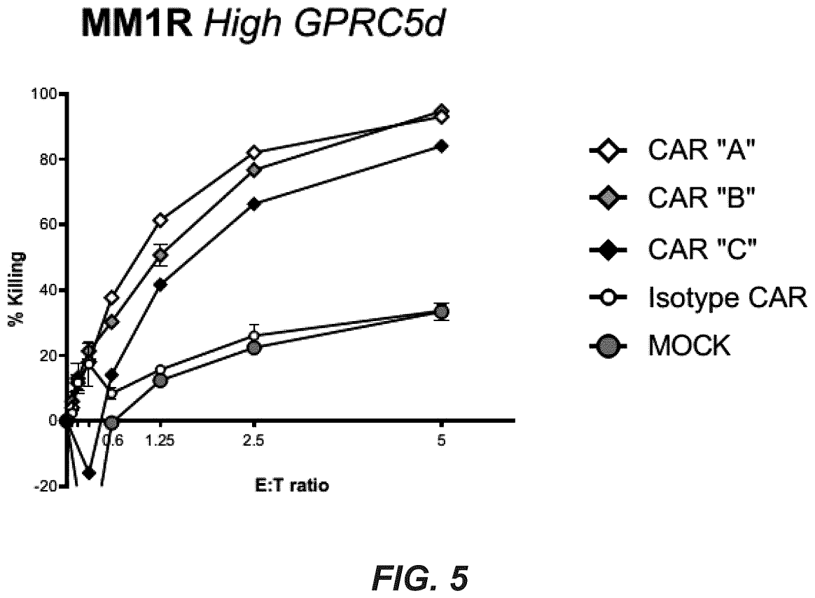

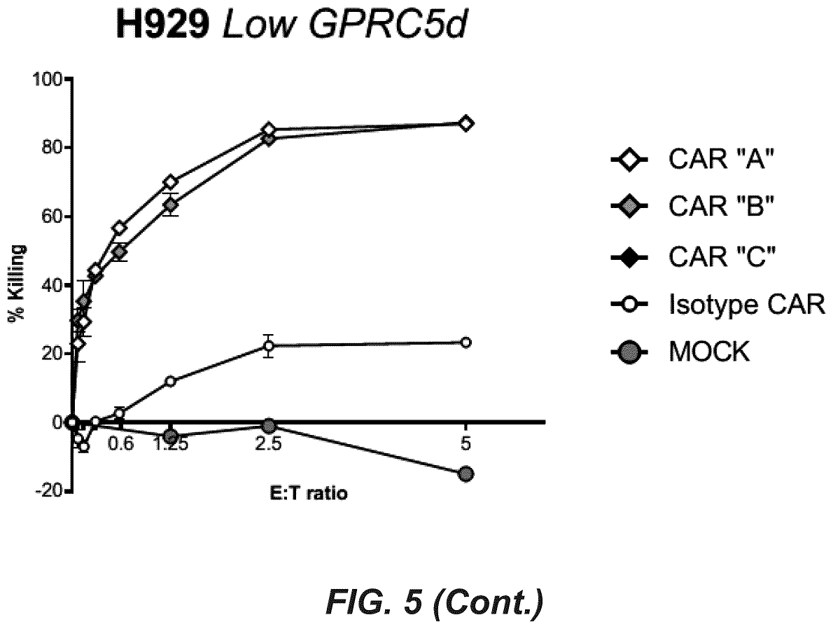

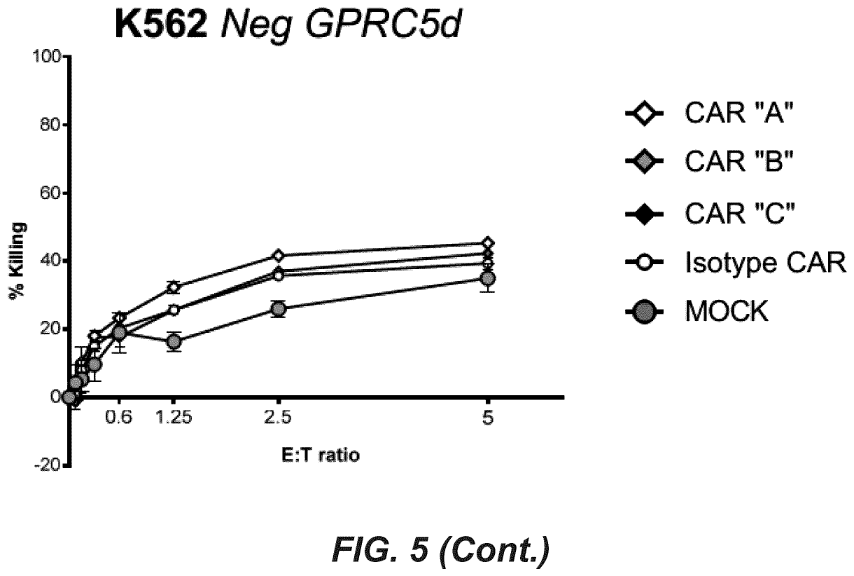

Cytotoxic potential of CAR-T cells on GPRC5D-expressing myeloma cells was assessed by flow-cytometry. Primary pan T cells (transiently expressing one of three CARs as described previously) were co-cultured at the indicated Effector:Target (E:T) ratios with fluorescently labeled myeloma cell lines, MM1R, H929 and K562 for eight hours, at which time co-cultures were stained with viability dye. Percent killing is the ratio of the absolute number of live (viability dye negative) target (CTV positive) cells remaining in the co-culture relative to the number of live targets cultured without CAR-T cells. (FIG. 5). CAR A, CAR B, CAR C, Isotype CAR and Mock T cells were co-cultured with GPRC5D+ cell lines MM1R and H929 or GPRC5D− K562 cells for eight hours. Various effector-to-target ratios were analyzed, showing that the CAR A, CAR B, and CAR C cells were able to induce cytotoxicity of MM1R cells. Increases in cytotoxicity correlated with higher effector-to-target ratios. The Isotype and Mock controls are included as controls and showed approximately 20-30% cytotoxicity. Similarly, CAR A and CAR B cells induced cytotoxicity of H929 cells. The CAR A and CAR B cells demonstrated similar cytotoxicity activity to Isotype and Mock cells. These data demonstrate the specificity of CAR A and CAR B cells to MM1R and H929 cells but not to K562 cells (GPRC5D).

### Example 5—Targeted Cytotoxicity of GPRC5D-Expressing Tumor Cell Lines: Multiple Myeloma Patient-Derived T-Cells

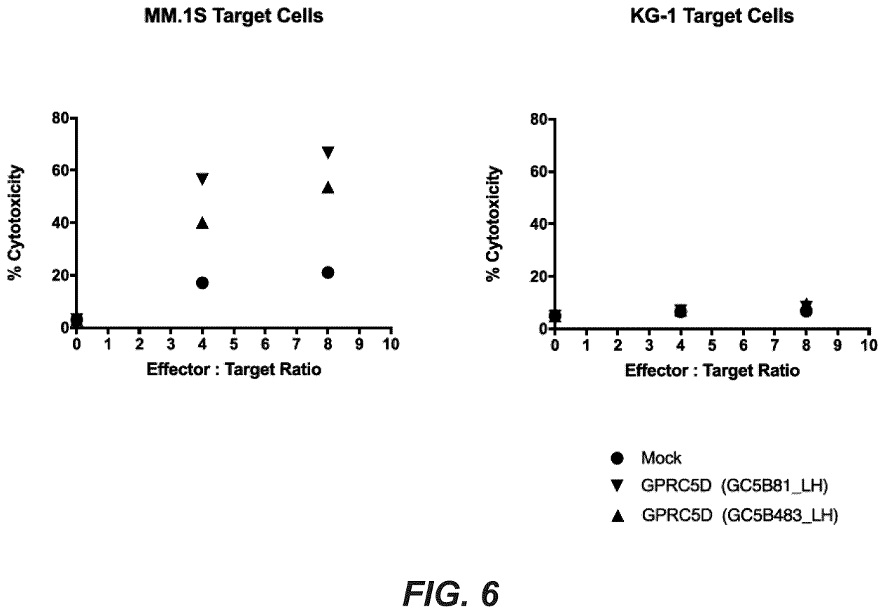

Cytotoxic potential of CAR-T cells on GPRC5D-expressing myeloma cells was assessed by flow-cytometry. Primary multiple myeloma patient T cells (transiently expressing one of two CARs as described previously) were co-cultured at the indicated Effector:Target (E:T) ratios with fluorescently labeled myeloma cell line MM.1S or control GPRC5D-negative cell line KG-1 for 48 hours, at which time co-cultures were stained with viability dye. Percent cytotoxicity was determined as percent fluorescently-labeled cells that stained positive for viability dye, indicating cell death. (FIG. 6). Primary multiple myeloma patient T cells transduced with either GC5B81_LH or GC5B483_LH both induced cytotoxicity of GPRC5D+MM.1 S cells with increasing amounts of effector cells compared to Mock T cells. There was no observable cytotoxicity with the GC5B81 LH or GC5B483_LH GPRC5D CAR-T cells when co-cultured with the GPRC5D− KG-1 cell line. These data show multiple myeloma T cells transduced with GPRC5D CAR's can elicit the killing of GPRC5D+ cells, but not GPRC5D cells.

### Example 6—Antigen-Stimulated Proliferative Response of α-GPRC5D CAR-T Cells

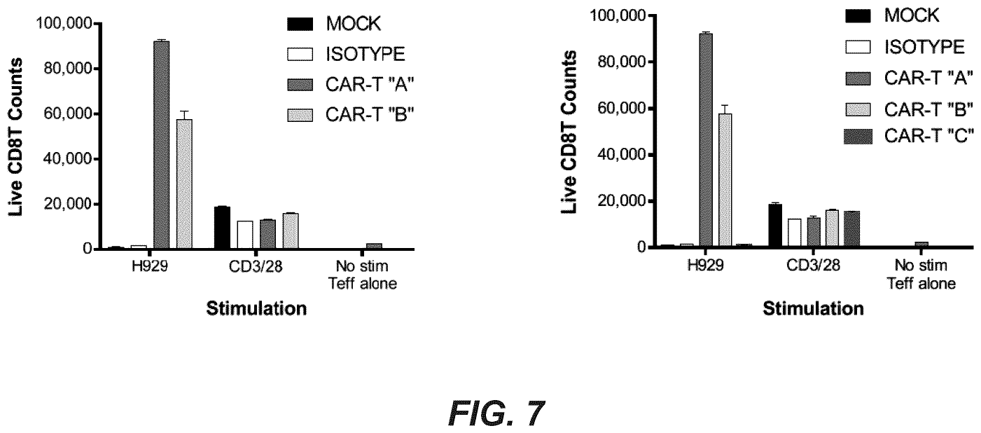

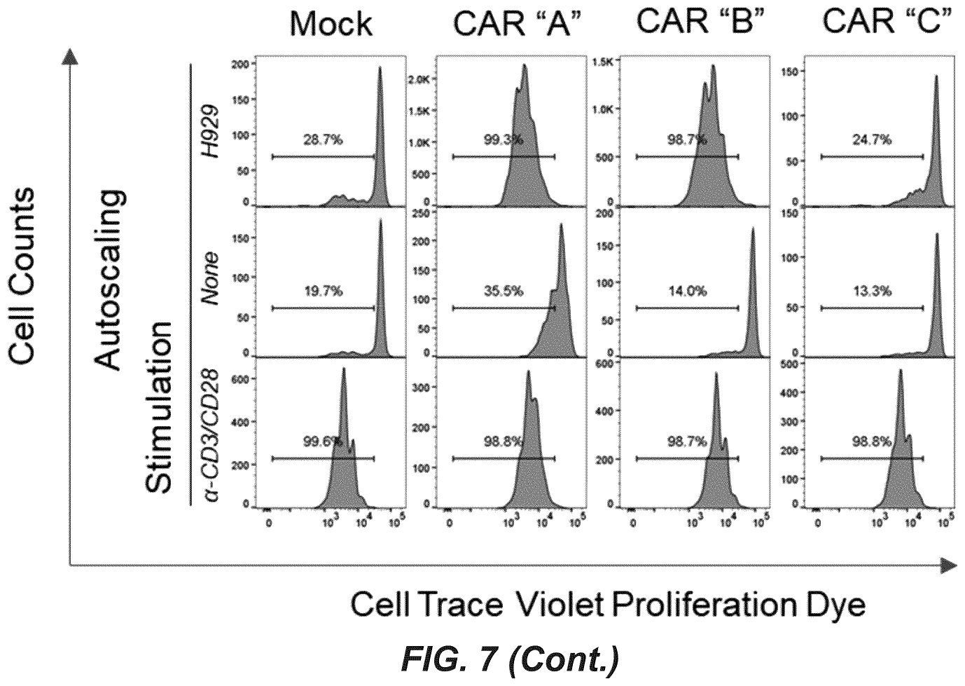

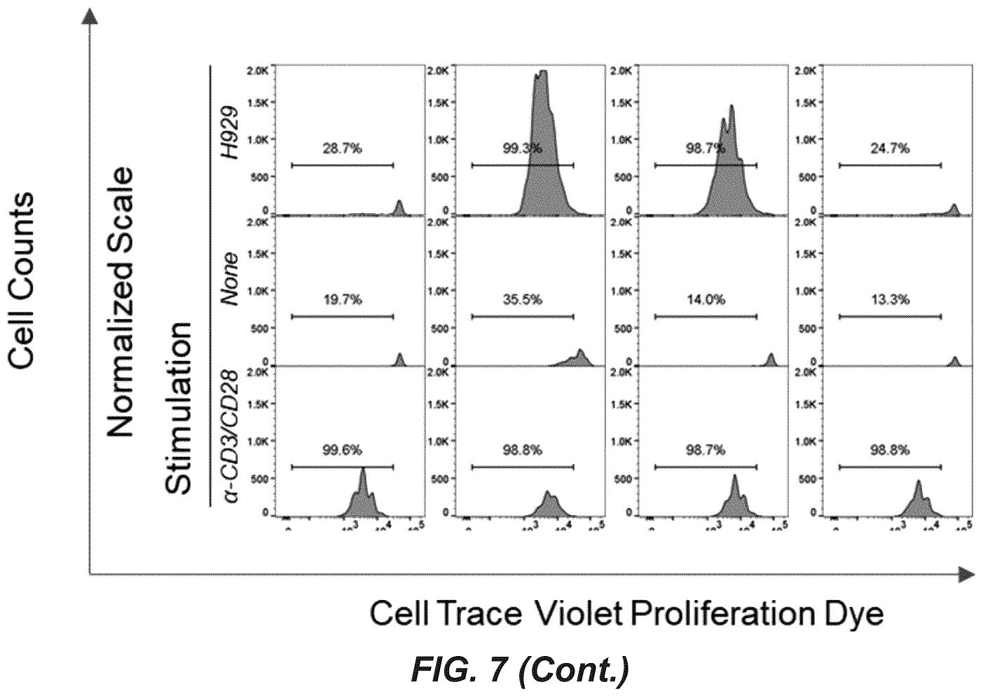

The proliferative capacity of α-GPRC5D CAR-T cells was assessed by flow-cytometry. Fluorescently labeled pan T cells (transiently expressing an α-GPRC5D CAR or isotype CAR control) were co-cultured at a 1 Effector: 1Target ratio with H929 for four days. Proliferation was measured as the absolute number of live (viability dye negative) CAR-T cells which had diluted fluorescent label (CTV). Proliferation in response to H929 was compared to CAR-T proliferation after four days of α-CD3/CD28 bead stimulation and CAR-T cultured alone (no stimulation control). (FIG. 7). Mock, Isotype CAR, CAR-T A, CAR-T B, and CAR-T C cells were co-cultured with GPRC5D+H929 cells, CD3/CD28 beads or without stimulations. After four days, CD4 and CD8 populations were analyzed for total live counts, reflecting the proliferative ability of the cells in response to GPRC5D antigen. In the CD4 population, CD3/CD28 beads were used as a positive control and all cell populations demonstrated an increase in CD4 events compared to no stimulation. Only the CAR-T A and CAR-T B cells showed a robust increase in counts in response to H929 cells compared to isotype and mock. Similar results were observed for the CD8 population; however, CAR-T C did not show a response in proliferation in response to H929 cells. Taken together, these data show that CAR-T A and CAR-T B cells proliferate in response to GPRC5D+H929 cells.

### Example 7—Expression of GPRC5D-CAR on Healthy Donor T Cells

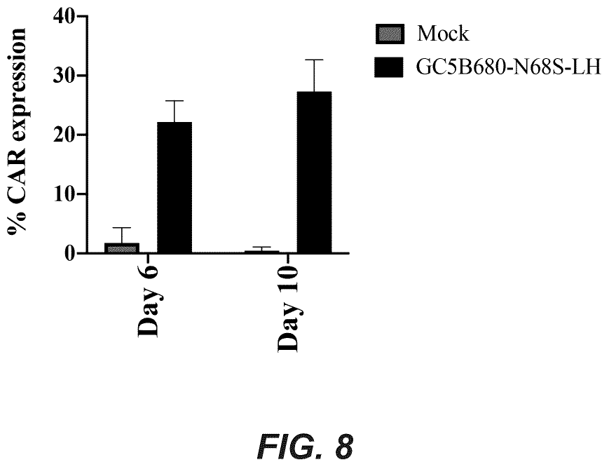

Six primary human pan T cells were activated and expanded using Miltenyi Biotec T cell TRANSACT system. 24 hours post-activation, T cells were transduced with lentivirus comprising a nucleotide sequence encoding the GPRC5D GC5B680-N68S-LH CAR (SEQ ID NO:90). The cells were allowed to expand for 12 days, with surface CAR expression measured by flow cytometry at Days 6 and 10 using a commercially available rabbit anti-human H+L detection antibody. The results are shown in FIG. 8, with the data representing the mean±SD from 6 healthy donors. The bars shaded gray (Mock) that are immediately to the left of the black bars (GCB6800-N68S-LH) represent background CAR detection using the commercial antibody in untransduced (mock) cells. The black bars represent GPRC5D GC5B680-N68S-LH transduced CAR-T cells.

### Example 8—Expression of CD4, CD8, and Memory Markers on GPRC5D CAR-T Cells

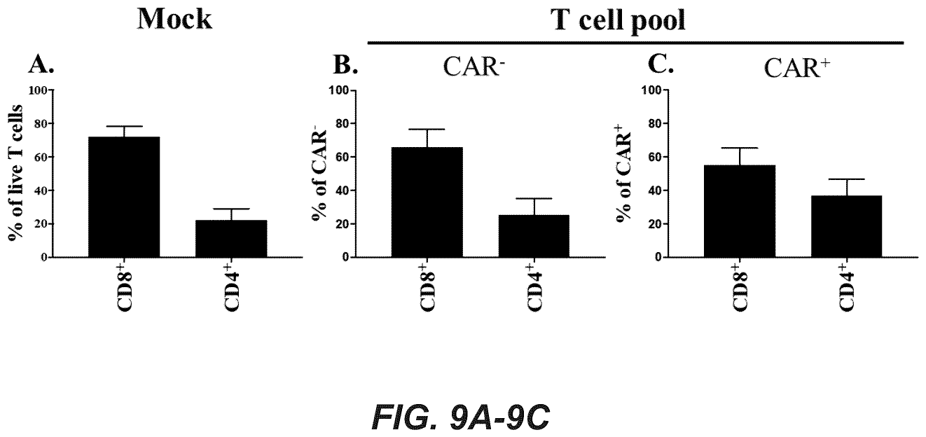

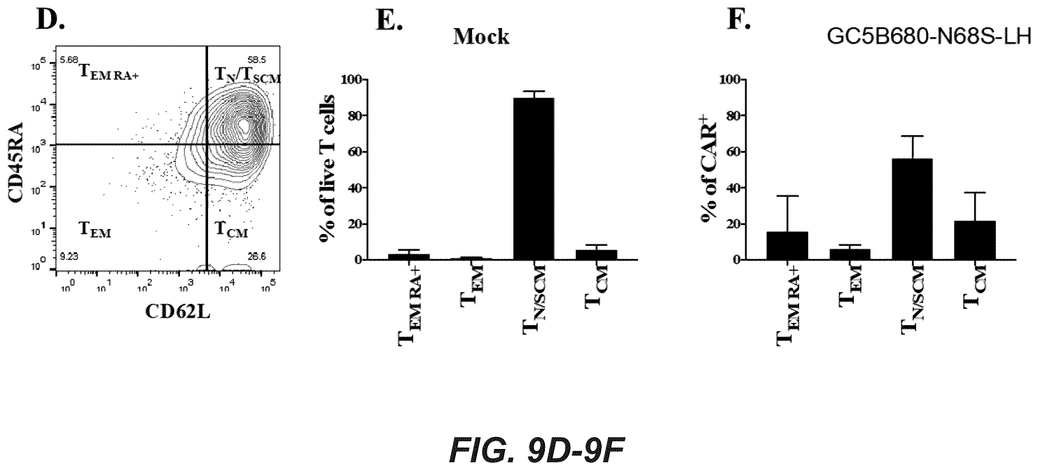

Pan T cells from six healthy donors were transduced with lentivirus comprising a nucleotide sequence encoding the GPRC5D GC5B680-N68S-LH CAR (GPRC5D CAR; SEQ ID NO:90). Mock (untransduced) cells and transduced cells (“T cell pool” including cells with surface CAR (CAR expressing or CAR⁺) and cells without surface CAR (CAR non-expressing or CAR⁻)] were evaluated for proportion of cells with surface CD4 (CD4⁺) and for proportion of cells with surface CD8 (CD8⁺). The data are shown in FIGS. 9A-9C. The data in FIG. 9C show that the GPRC5D CAR was detected in both CD4⁺ and CD8⁺ T cells, as expected. FIGS. 9A-9C also show that the CD4:CD8 ratio was similar among all cell populations evaluated, including CAR⁴ cells, CAR⁻ cells, and mock cells.

T cell subtypes in the CAR-T population were determined using two surface markers CD45RA and CD62L to differentiate between four memory cell populations: effector memory RA⁺ T cells (T_{EM RA+}), effector memory cells T_{EM}, naïve T cells and memory stem cells T_{N/SCM}, and central memory T cells (T_{CM}). The data are shown in FIGS. 9D-9F. FIG. 9D is a flow cytogram representative of one donor sample highlighting a gating strategy for distinguishing the four different memory populations using the two surface markers. FIGS. 9E and 9F show that GPRC5D GC5B680-N68S-LH was expressed mostly on T_{N/SCM} and T_{CM} cells, which are known to have high proliferative capacity, survival, and therapeutic efficacy.

### Example 9—Assay of GPRC5D CAR-T Cytotoxicity at 6 Hours and 24 Hours

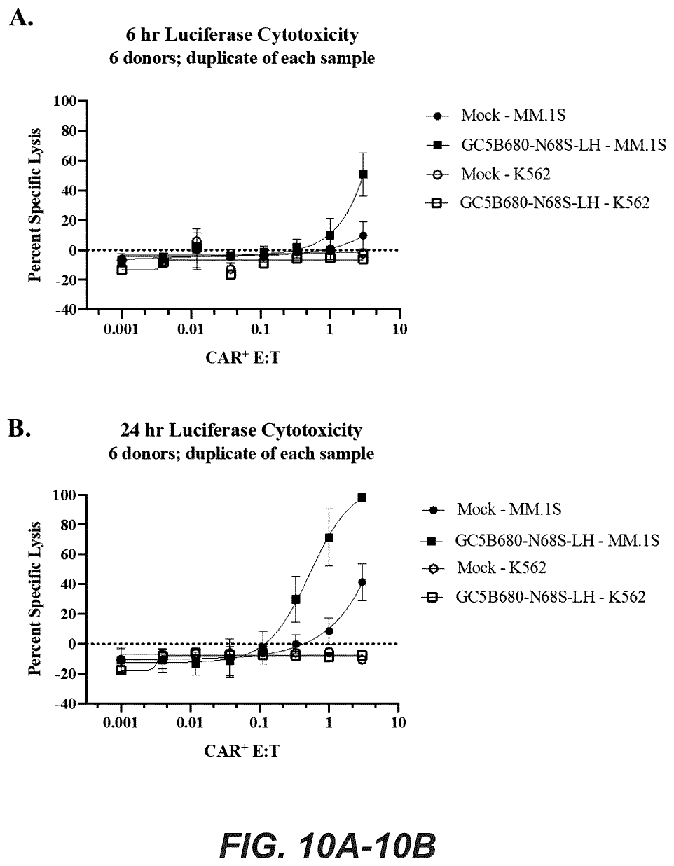

GPRC5D GC5B680-N68S-LH transduced T cells from one of six healthy donors, or untransduced (Mock) T cells, were added at various E:T ratios to (i) cultures of GPRC5D⁺ MM.1S tumor cells comprising a luciferase transgene or to (ii) cultures of GPRC5D⁻ K562 tumor cells comprising a luciferase transgene. Cytotoxicity was assessed at each E:T ratio after 6 hours or 24 hours of incubation. The data are shown in FIG. 10A for the 6 hour time point, and in FIG. 10B for the 24 hour time point. The percent specific lysis was calculated (i) by measuring luciferase signal in tumor cells incubated in the presence of CAR-Ts at 6 or 24 hours divided by luciferase signal in the tumor cells incubated alone at the same time points and multiplied by 100 and (ii) by subtracting that number from 100. The equation is: 100−[(CAR-T+tumor luminescence)/(average tumor alone luminescence)]×100. The dotted line highlights zero lysis. Negative lysis is indicative of cell growth.

### Example 10—Assay of GPRC5D CAR-T Cytotoxicity at 48 Hours

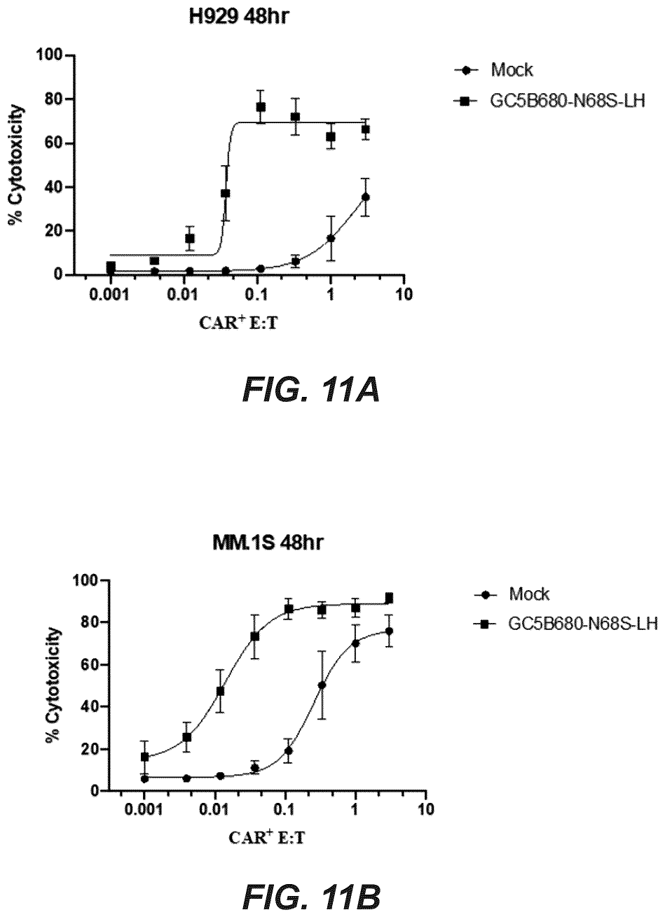

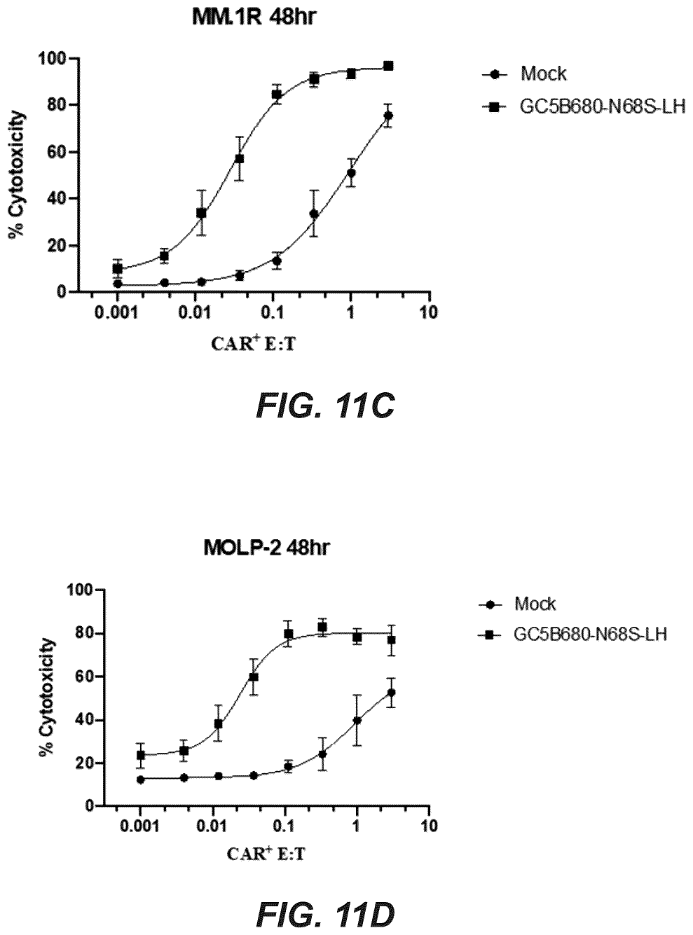

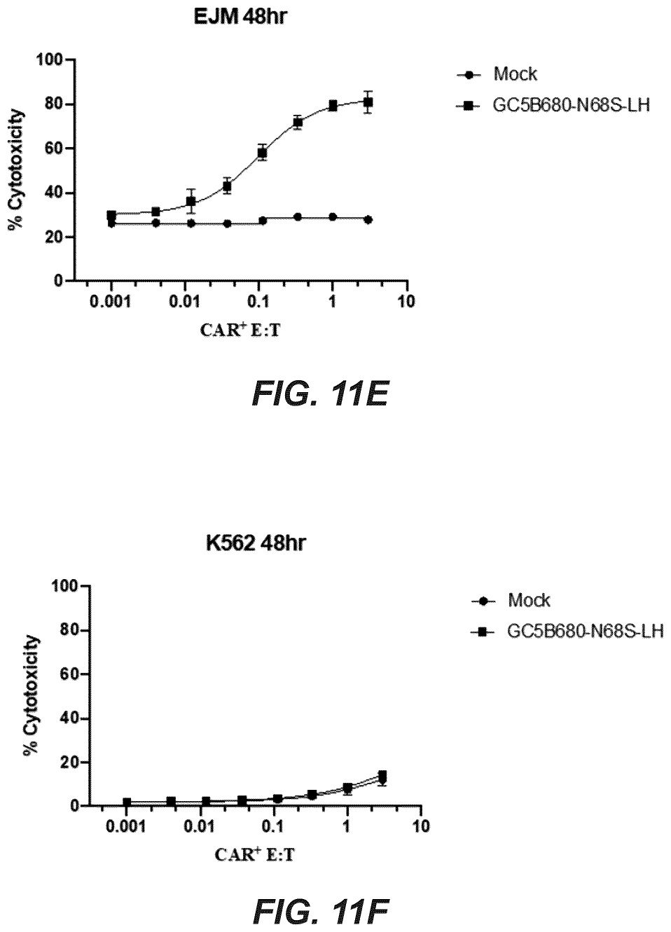

GPRC5D GC5B680-N68S-LH transduced and untransduced (Mock) T cells were prepared from healthy donors. Either (i) GPRC5D GC5B680-N68S-LH transduced T cells or (ii) untransduced (Mock) T cells were added at various E:T ratios to carboxyfluorescein succinimidyl ester (CFSE) labeled GPRC5D⁺ H929, GPRC5D⁺ MM.1S, GPRC5D⁺ MM.1R, GPRC5D⁺ MOLP-2, GPRC5D⁺ EJM and GPRC5D K562 cells. After 48 hours of incubation, the cells were stained with a live/dead-dye and evaluated for percentage of dead and CFSE⁺ cells (% cytotoxicity). The percent cytotoxicity was plotted against a log transformed x-axis (E:T ratios) with a 4-parameter non-linear regression curve fit, with data shown in FIGS. 11A-11F as mean±SD. Mock negative control T cells did induce some degree of cytotoxicity in all GPRC5D⁺ cell lines other than EJM, but to a lesser degree than GPRC5D GC5B680-N68S-LH cells, especially at the higher E:T ratios. Without wishing to be bound by theory, the reduced cytotoxicity likely arose from an allogeneic response. In addition, cytotoxicity of the GPRC5D CAR cells varied depending on cell line.

### Example 11—Assay of GPRC5D CAR-T Activation at 48 Hours

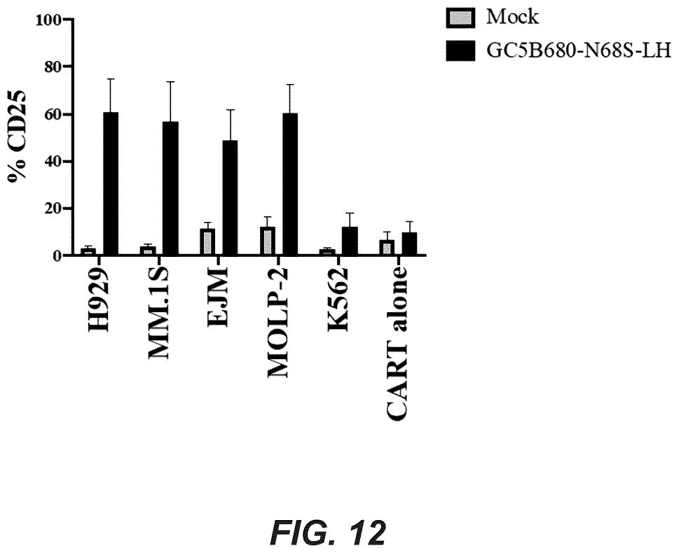

GPRC5D GC5B680-N68S-LH transduced and untransduced (Mock) T cells were prepared from healthy donors. GPRC5D GC5B680-N68S-LH transduced or untransduced (Mock) T cells were added at a 3:1 E:T ratio to carboxyfluorescein succinimidyl ester (CFSE) labeled GPRC5D⁺ H929, GPRC5D⁺ MM.1S, GPRC5D MOLP-2, GPRC5D⁺ EJM, and GPRC5D⁺ K562 cells. After 48 hours of incubation, the cells were stained with a live/dead-dye, anti-CD25, anti-CD3, and anti-CAR visual markers. The cells were then evaluated for surface CD25 expression. The data are expressed in FIG. 12 as the mean±SD. As shown in FIG. 12, less than 13% background surface CD25 expression was observed in mock cells (i.e., no activation). In contrast, GC5B680-N68S-LH CAR-T cells were activated in the presence of GPRC5D⁺ cell lines, as shown by heightened CD25 expression levels. In both (i) mock and (ii) GC5B680-N68S-LH CAR-T cells only, low levels of surface CD25 expression was observed upon incubation with K562 cells or upon incubation alone.

### Example 12—Assay of GPRC5D CAR-T Polyfunctionality

GC5B680-N68S-LH transduced (CAR-T) cells were incubated with either PMA/ionomycin (as a positive control), a GPRC5D cell line H929 (H929), GPRC5D K562 (K562) cells (CFSE-labeled), or alone, for 18 hours. The cells were incubated at an E:T ratio of 1:2 when incubated in the presence of H929 or K562 cells. The cells were then first surface-stained with a live/dead dye, anti-CD3, and anti-idiotype followed by fixation, permeabilization, and intracellularly staining with anti-IFN-γ, anti-IL-2, and anti-TNF-α. Stained cells were then analyzed by flow cytometry. The percentages of individual cytokine levels were calculated following analysis of flow cytograms using Boolean (hierarchical) gating, with the percentages represented in Tables 3-6 below. Also in Tables 3-6, the levels of polyfunctional cytokine release for each group are highlighted in gray with the sums of averages represented.

TABLE 3
Cytokine expression in CAR-T cells incubated in the presence of H929

| | | | Polyfunctional Populations | | | | | | | |
|---|---|---|---|---|---|---|---|---|---|---|
| | | | IFNγ+ | IFNγ+ | IFNγ+ | IFNγ− | IFNγ+ | IFNγ− | IFNγ− | IFNγ− |
| | | T cell | IL2+ | IL2+ | IL2− | IL2+ | IL2− | IL2+ | IL2− | IL2− |
| CAR-T | Target | donor ID | TNFα+ | TNFα− | TNFα+ | TNFα+ | TNFα− | TNFα− | TNFα+ | TNFα− |
| GC5B680- | H929 | D204071 | 2.1 | 0.2 | 4.4 | 18.1 | 0.8 | 1.8 | 43.2 | 29.4 |
| N68S-LH | | | | | | | | | | |
| GC5B680- | H929 | D270235 | 4.1 | 0.5 | 8.5 | 10.9 | 3.8 | 2.2 | 31.4 | 38.6 |
| N68S-LH | | | | | | | | | | |
| GC5B680- | H929 | 110039522 | 1.3 | 0.1 | 1.8 | 19.1 | 0.5 | 0.5 | 42.2 | 34.6 |
| N68S-LH | | | | | | | | | | |
| GC5B680- | H929 | D202896 | 13.5 | 0.0 | 30.7 | 6.8 | 2.1 | 0.4 | 27.1 | 19.5 |
| N68S-LH | | | | | | | | | | |
| GC5B680- | H929 | D328058 | 7.0 | 0.1 | 12.2 | 18.2 | 0.8 | 0.2 | 32.9 | 28.7 |
| N68S-LH | | | | | | | | | | |
| GC5B680- | H929 | D204395 | 4.3 | 0.0 | 4.6 | 30.5 | 0.4 | 0.3 | 37.9 | 22.1 |
| N68S-LH | | | | | | | | | | |
| | | Percent | 33.1 | | | | | | | |
| | | polyfunctional | | | | | | | | |
| | | H929 | | | | | | | | |

TABLE 4
Cytokine expression in CAR-T cells incubated in the presence of K562

| | | | Polyfunctional Populations | | | | | | | |
|---|---|---|---|---|---|---|---|---|---|---|
| | | | IFNγ+ | IFNγ+ | IFNγ+ | IFNγ− | IFNγ+ | IFNγ− | IFNγ− | IFNγ− |
| | | T cell | IL2+ | IL2+ | IL2− | IL2+ | IL2− | IL2+ | IL2− | IL2− |
| CAR-T | Target | donor ID | TNFα+ | TNFα− | TNFα+ | TNFα+ | TNFα− | TNFα− | TNFα+ | TNFα− |
| GC5B680- | K562 | D204071 | 0.0 | 0.0 | 0.5 | 0.5 | 1.5 | 3.2 | 5.7 | 88.5 |
| N68S-LH | | | | | | | | | | |
| GC5B680- | K562 | D270235 | 0.2 | 0.5 | 2.0 | 1.1 | 2.7 | 3.0 | 7.7 | 83.0 |
| N68S-LH | | | | | | | | | | |
| GC5B680- | K562 | 110039522 | 0.1 | 0.3 | 0.3 | 0.2 | 1.0 | 1.1 | 3.6 | 93.4 |
| N68S-LH | | | | | | | | | | |
| GC5B680- | K562 | D202896 | 0.2 | 0.2 | 1.3 | 0.5 | 2.3 | 4.5 | 3.7 | 87.4 |
| N68S-LH | | | | | | | | | | |
| GC5B680- | K562 | D328058 | 0.2 | 0.1 | 1.4 | 0.3 | 1.6 | 2.1 | 6.1 | 88.1 |
| N68S-LH | | | | | | | | | | |
| GC5B680- | K562 | D204395 | 0.1 | 0.2 | 0.5 | 0.5 | 0.7 | 3.1 | 9.4 | 85.5 |
| N68S-LH | | | | | | | | | | |
| | | Percent | 1.9 | | | | | | | |
| | | polyfunctional | | | | | | | | |
| | | K562 | | | | | | | | |

TABLE 5
Cytokine expression in CAR-T cells incubated alone

| | | | Polyfunctional Populations | | | | | | | |
|---|---|---|---|---|---|---|---|---|---|---|
| | | | IFNγ+ | IFNγ+ | IFNγ+ | IFNγ− | IFNγ+ | IFNγ− | IFNγ− | IFNγ− |
| | | T cell | IL2+ | IL2+ | IL2− | IL2+ | IL2− | IL2+ | IL2− | IL2− |
| CAR-T | Target | donor ID | TNFα+ | TNFα− | TNFα+ | TNFα+ | TNFα− | TNFα− | TNFα+ | TNFα− |
| GC5B680- | CART | D204071 | 0.0 | 0.4 | 0.7 | 0.1 | 2.0 | 3.2 | 4.3 | 89.2 |
| N68S-LH | alone | | | | | | | | | |
| GC5B680- | CART | D270235 | 0.4 | 0.8 | 1.8 | 0.4 | 4.1 | 3.7 | 6.5 | 82.4 |
| N68S-LH | alone | | | | | | | | | |
| GC5B680- | CART | 110039522 | 0.1 | 0.4 | 0.2 | 0.2 | 1.7 | 1.4 | 1.9 | 94.0 |
| N68S-LH | alone | | | | | | | | | |
| GC5B680- | CART | D202896 | 0.1 | 0.6 | 1.8 | 0.4 | 2.4 | 6.8 | 2.1 | 86.0 |
| N68S-LH | alone | | | | | | | | | |
| GC5B680- | CART | D328058 | 0.2 | 0.4 | 1.0 | 0.3 | 1.4 | 3.2 | 3.9 | 89.6 |
| N68S-LH | alone | | | | | | | | | |
| GC5B680- | CART | D204395 | 0.1 | 0.3 | 0.7 | 0.5 | 1.6 | 5.0 | 7.2 | 84.5 |
| N68S-LH | alone | | | | | | | | | |
| | | Percent | 2.0 | | | | | | | |
| | | polyfunctional | | | | | | | | |
| | | CAR-T alone | | | | | | | | |

TABLE 6
Cytokine expression in CAR-T cells incubated in the presence of PMA/lonomycin

| | | | Polyfunctional Populations | | | | | | | |
|---|---|---|---|---|---|---|---|---|---|---|
| | | | IFNγ+ | IFNγ+ | IFNγ+ | IFNγ− | IFNγ+ | IFNγ− | IFNγ− | IFNγ− |
| | | T cell | IL2+ | IL2+ | IL2− | IL2+ | IL2− | IL2+ | IL2− | IL2− |
| CAR-T | Target | donor ID | TNFα+ | TNFα− | TNFα+ | TNFα+ | TNFα− | TNFα− | TNFα+ | TNFα− |
| GC5B680- | PMA/ | D204071 | 10.8 | 0.1 | 6.0 | 62.7 | 0.2 | 1.5 | 15.8 | 2.9 |
| N68S-LH | Ionomycin | | | | | | | | | |
| GC5B680- | PMA/ | D270235 | 31.0 | 0.2 | 16.7 | 43.9 | 0.3 | 0.4 | 7.0 | 0.6 |
| N68S-LH | Ionomycin | | | | | | | | | |
| GC5B680- | PMA/ | 110039522 | 10.5 | 0.0 | 2.8 | 68.8 | 0.0 | 0.4 | 16.1 | 1.5 |
| N68S-LH | Ionomycin | | | | | | | | | |
| GC5B680- | PMA/ | D202896 | 42.8 | 0.3 | 29.1 | 20.0 | 0.5 | 0.1 | 6.2 | 1.0 |
| N68S-LH | Ionomycin | | | | | | | | | |
| GC5B680- | PMA/ | D328058 | 33.6 | 0.1 | 15.4 | 42.1 | 0.2 | 0.2 | 7.5 | 0.9 |
| N68S-LH | Ionomycin | | | | | | | | | |
| GC5B680- | PMA/ | D204395 | 21.2 | 0.0 | 6.1 | 59.3 | 0.4 | 1.0 | 10.7 | 1.3 |
| N68S-LH | Ionomycin | | | | | | | | | |
| | | Percent | 87.3 | | | | | | | |
| | | polyfunctional | | | | | | | | |
| | | PMA/Ionomycin | | | | | | | | |

### Example 13—Assay of GPRC5D CAR-T Proliferation

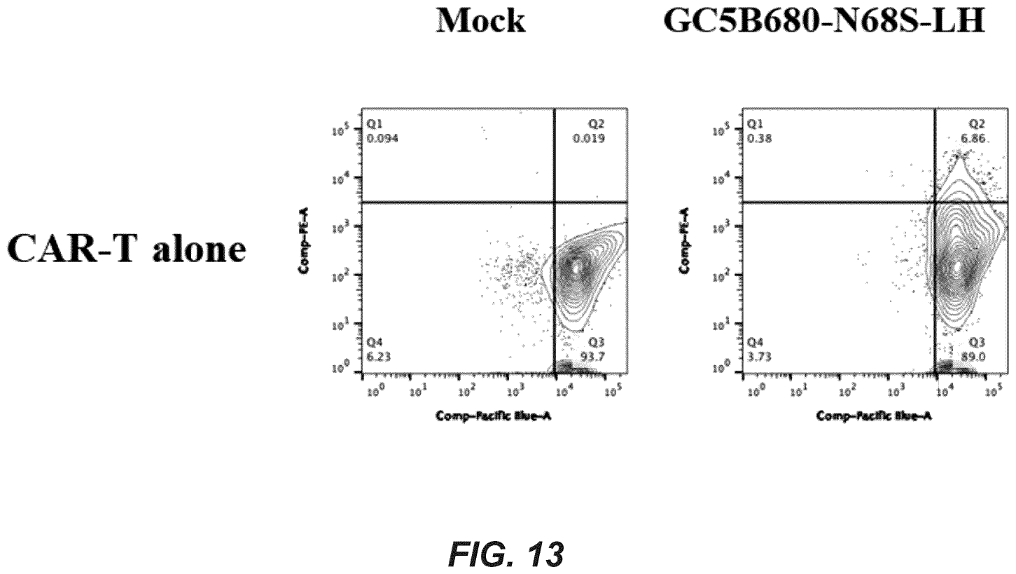

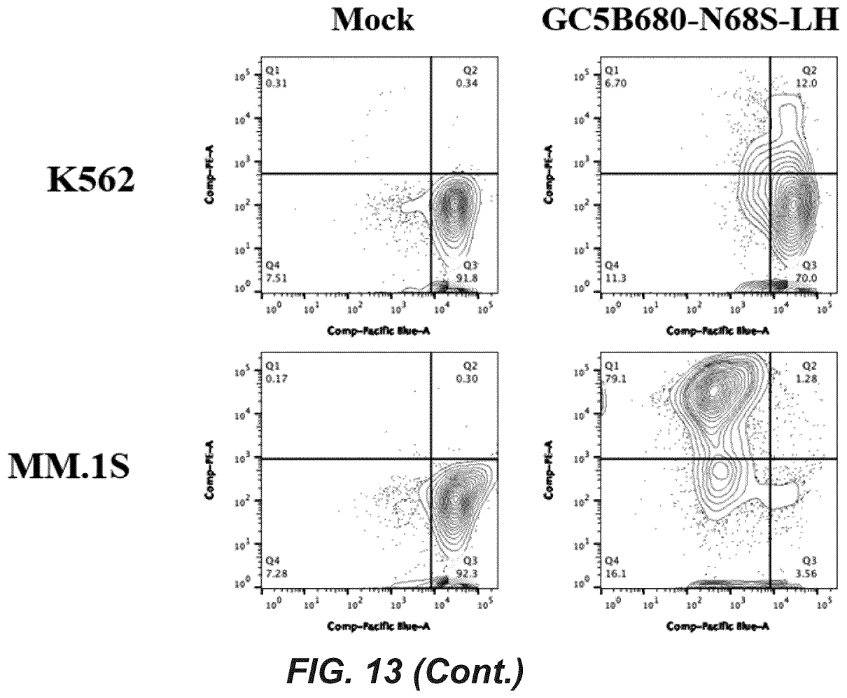

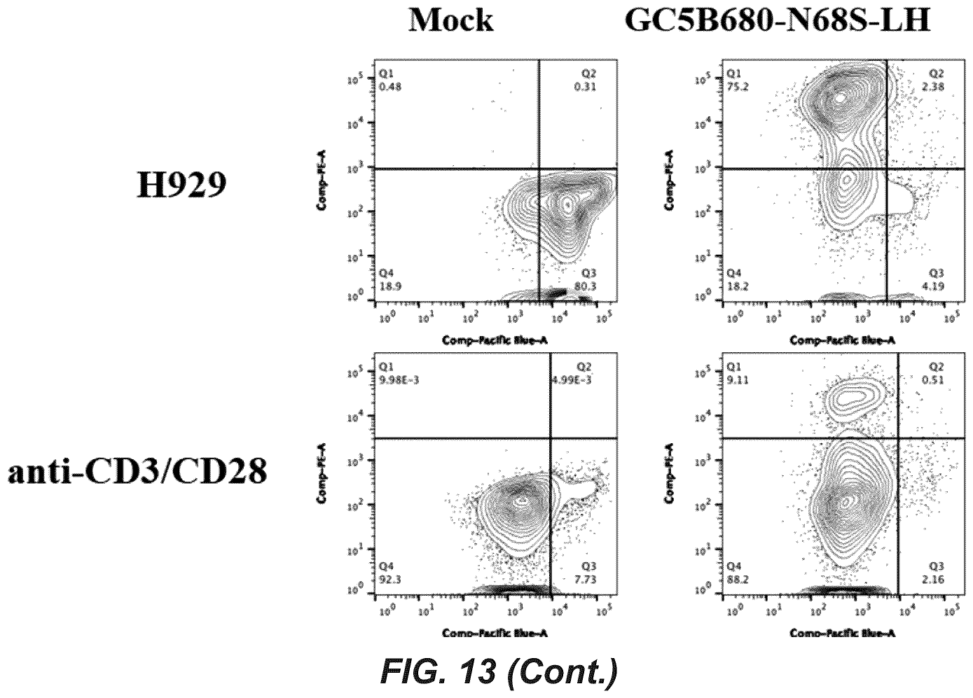

GC5B680-N68S-LH cells were prepared using cells from one donor. The (i) GC5B680-N68S-LH transduced cells and (ii) mock cells were incubated in the presence or absence of GPRC5D⁺ cells (MM.1S and H929). Mock (untransduced) cells were used as a negative control. The extent of cell proliferation was measured following a 6 day incubation by assaying the extent of dilution of CELLTRACE Violet (CTV) dye concentration in cells, with the quantity represented by the X axis of the graphs of FIG. 13. CAR⁺ cells were stained using an anti-idiotype antibody, with the degree of staining represented by the Y axis of the graphs of FIG. 13.

As shown in FIG. 13, mock and CTV-labeled CAR T cells, alone or in the presence of GPRC5D⁻ K562 cells, demonstrated little to no cell proliferation as seen from low to zero dilution of the CTV dye. Agonist CD3/CD28 beads (that served as a positive control) resulted in proliferation of all T cell populations evaluated, as predicted. When CAR-T cells were incubated with GPRC5D target cells (H929 or MM.1S), proliferation was primarily enriched in transduced cells expressing GC5B680-N68S-LH on their surface (CAR⁺) (as shown in the upper quadrants in the graphs of FIG. 13). This result suggested that GC5B680-N68S-LH cell proliferation is enhanced in the presence of GPRC5D⁺ target cells. The data shown in FIG. 13 is representative of similar observations made using cells prepared from other donors.

### Example 14—Assay of GPRC5D CAR-T Proliferation

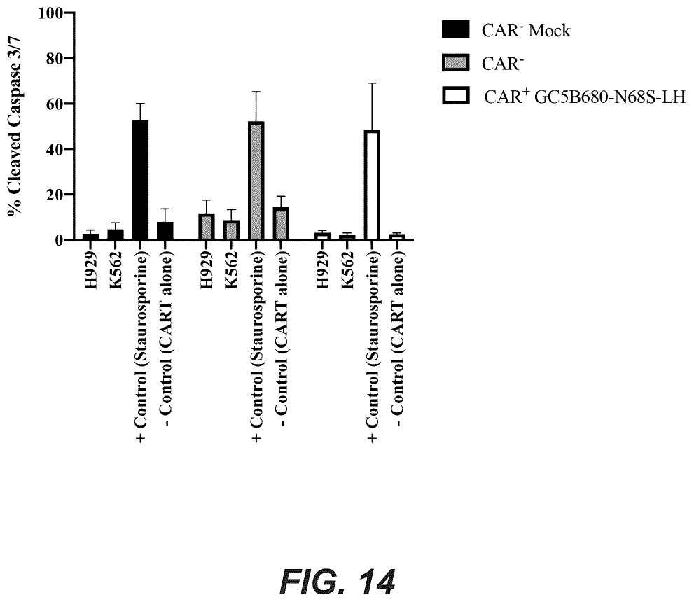

GC5B680-N68S-LH (CAR-T) cells were incubated with either positive control staurosporine, GPRC5D⁺ H929 cells, GPRC5D⁻ K562 cells, or alone, for 24 hours. An E:T ratio of 1:2 was used for incubations of GC5B680-N68S-LH (CAR-T) cells with H929 or K562 cells. The cells were then stained with a live/dead dye, anti-CD3, anti-idiotype, and anti-cleaved caspase 3/7 dye. The stained cells were analyzed by flow cytometry. The data are expressed in the graph of FIG. 14. As shown in FIG. 14, staurosporine induced cleaved caspase 3/7 staining in both CAR⁺ and CAR⁻ T cell populations. Also, there were 50-65% positive cells starting from 4 hours after the 24 hour timepoint. Neither CAR⁺ nor CAR T cell populations demonstrated significant differences in caspase 3/7 levels between negative control incubations (K562 and CART alone) and incubations in the presence of H929.

### Example 15—Assay of Efficacy of GC5B680-N68S-LH, GC5B680-N68S-HL, GP5B83 N24T N31S-LH, and GP5B83 N24T N31S-HL on Established MM.1S Human MM Xenografts in NSG-B2M Mice

The anti-tumor efficacy of GC5B680-N68S-LH (SEQ ID NO:86), GC5B680-N68S-HL (SEQ ID NO:85), GP5B83_N24T_N31S-LH (SEQ ID NO:84), and GP5B83_N24T_N31S-HL (SEQ ID NO:83) transduced T cells (CAR-T cells) was evaluated in the established subcutaneous (sc) MM.1S human multiple myeloma (MM) xenograft model in female NSG B2m (alternatively referred to as NOD-scid I12rg^{null} B2m^{null}, or as NOD-scid gamma B2m). The (i) GC5B680-N68S-LH, (ii) GC5B680-N68S-HL, (iii) GP5B83_N24T_N31S-LH, or (iv) GP5B83_N24T_N31S-HL CAR-T cells were administered at a dose of 1×10⁶ CAR⁺ cells or 5×10⁶ CAR⁺ cells intravenously (iv) on Day 13 for a total of one dose. Three groups of MM.1S xenograft-bearing mice were assessed: (i) mice treated with PBS, (ii) mice that underwent mock transduction, and (iii) mice that were transduced with GPRC5D. The percentage delta tumor growth inhibition (% ΔTGI) of the three groups of mice bearing SC (subcutaneous) MM.1S xenografts was calculated on Day 26 post tumor implantation using the formula: ([(TVc−TVc0)−(TVt−TVt0)]/(TVc−TVc0))×100 where ‘TVc’ is the mean tumor burden of a given control group, ‘TVc0’ is the mean initial tumor burden of a given control group, ‘TVt’ is the mean tumor burden of the treated group, and ‘TVt0’ is the mean initial tumor burden of the treated group.

Statistical significance was calculated using the linear mixed-effects analysis in R, with treatment and time as fixed effects and animal as random effect. Logarithmic transformation (base 10) was performed if individual longitudinal response trajectories were not linear. The information derived from this statistical model was used to make pairwise treatment comparisons to the PBS control or mock transduced groups, with p values considered statistically significant when <0.05.

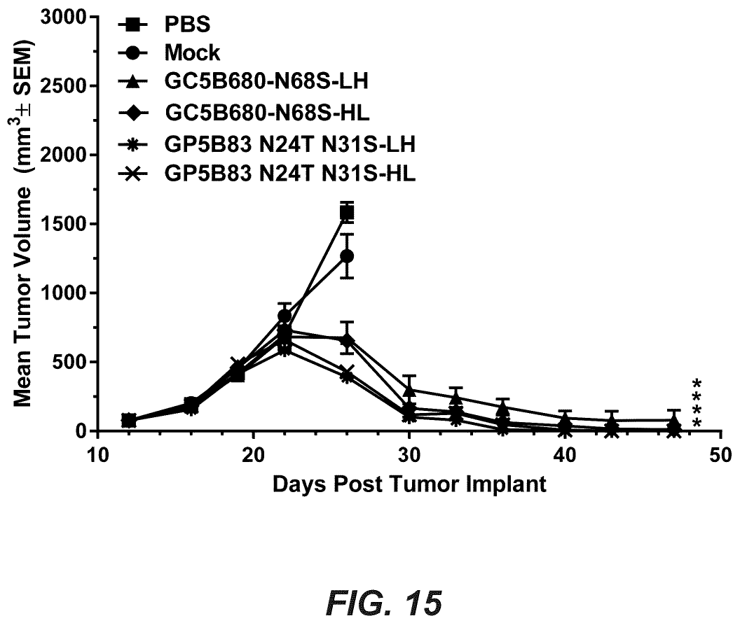

All four GPRC5D-directed CAR-T constructs elicited statistically significant % ΔTGI at both 1×10⁶ and 5×10⁶ CAR⁺ T cell dose levels as compared with PBS and Mock CAR-T controls, as summarized in Table 7 and FIG. 15. Complete responses (complete tumor regression, with no palpable tumor) were assessed on Day 47 post-tumor implantation.

TABLE 7
Summary of GPRC5D-directed CAR-T Efficacy on Established
MM.1S Human MM Xenografts in NSG-B2M Mice

| | | Total dose of | | |
|---|---|---|---|---|
| | Dose | T cells (counting | | CRs |
| | of CAR⁺ | CAR⁺ and | % | (n of |
| Construct | cells | CAR⁻ cells) | ΔTGI | group) |
| Mock | — | 28.6 × 10⁶ | 21% | 0/10 |
| GC5B680-N68S-LH | 1 × 10⁶ | 5.71 × 10⁶ | 50% | 8/10 |
| | 5 × 10⁶ | 28.6 × 10⁶ | 97% | 10/10 |
| GC5B680-N68S-HL | 1 × 10⁶ | 5.71 × 10⁶ | 52% | 8/10 |
| | 5 × 10⁶ | 28.6 × 10⁶ | 95% | 10/10 |
| GP5B83 N24T | 1 × 10⁶ | 5.71 × 10⁶ | 74% | 9/10 |
| N31S-LH | 5 × 10⁶ | 28.6 × 10⁶ | 100% | 10/10 |
| GP5B83 N24T | 1 × 10⁶ | 5.71 × 10⁶ | 70% | 10/10 |
| N31S-HL | 5 × 10⁶ | 28.6 × 10⁶ | 99% | 10/10 |

p < 0.05 vs. PBS and Mock CAR-T controls except where noted as not significant (ns).
CR = complete response

### Example 16 Efficacy of GC5B680-N68S-LH. GC5B680-N68S-HL. GP5B83 N24T N31S-LH, and GP5B83 N24T N31S-HL on Disseminated H929 IV Human MM Xenografts in NSG Mice

The efficacy of GC5B680-N68S-LH (SEQ ID NO:86; encoded by nucleotide of SEQ ID NO: 90), GC5B680-N68S-HL (SEQ ID NO:85; encoded by nucleotide of SEQ ID NO: 89), GP5B83_N24T_N31S-LH (SEQ ID NO:84; encoded by nucleotide of SEQ ID NO: 88), and GP5B83_N24T_N31S-HL (SEQ ID NO:83; encoded by nucleotide of SEQ ID NO: 87) transduced T cells was evaluated on a disseminated (iv) H929 human MM model in female NSG™ (alternatively referred to as NOD scid gamma, or as NOD.Cg-Prkdc^{scid} I12rg^{tm1Wj1}/SzJ) mice. In this example, GC5B680-N68S-LH, GC5B680-N68S-HL, GP5B83_N24T_N31S-LH, or GP5B83_N24T_N31S-HL expressing (CAR⁺) T cells were administered at a single iv dose of 1×10⁶ CAR⁺ cells or 5×10⁶ CAR⁺ cells on Day 9 post-tumor engraftment.

A survival assessment was performed in which the percentage survival at various study days was plotted against the study days. Hind-limb paralysis or other clinical signs of excessive disseminated tumor burden were used as surrogate endpoints for death. Median survival was determined using Kaplan-Meier survival analysis. Percent ILS (% ILS) was defined as the difference between median survival of the treated versus control group, and was calculated as ILS=([MSt−MSc]/MSc)×100 where ‘MSc’ is the median survival of a given control group and ‘MSt’ is the median survival of a particular treatment group. Animals failing to reach the surrogate endpoint due to adverse clinical signs (such as ulcerated tumors, body weight loss, etc.) or death unrelated to treatment, were censored for the survival assessment. Survival was graphically represented using a Kaplan-Meier curve and evaluated by log-rank (Mantel-Cox) test using GraphPad Prism software (version 7.0).

Median 50% survival was reached by Day 52 post tumor implantation (43 days post CAR⁺ injection) for PBS-treated control mice. Median 50% survival was reached by Day 44 post tumor implantation (35 days post CAR-T injection) for Mock CAR-T cell treated mice. Median 50% survival was reached after Day 111 (102 days post CAR-T injection) for mice treated with GC5B680-N68S-LH CAR⁺ cells administered at a dose of 1×10⁶. Median 50% survival was reached at Day 63.5 (54.5 days post CAR-T injection) for mice treated with GC5B680-N68S-HL CAR⁺ cells administered at a dose of 1×10⁶ cells respectively. Median 50% survival was reached Day 93.5 (84.5 days post CAR-T injection) for mice treated with GP5B83_N24T_N31S-LH CAR⁺ cells administered at a dose of 1×10⁶ cells. Median 50% survival was reached after Day 1 I1 (102 days post CAR-T injection) for mice treated with GP5B83_N24T_N31S-HL CAR⁺ cells administered at a dose of 1×10⁶ cells. The percent increased lifespan (ILS) was assessed via Kaplan-Meier curve and evaluated by log-rank Mantel-Cox test.

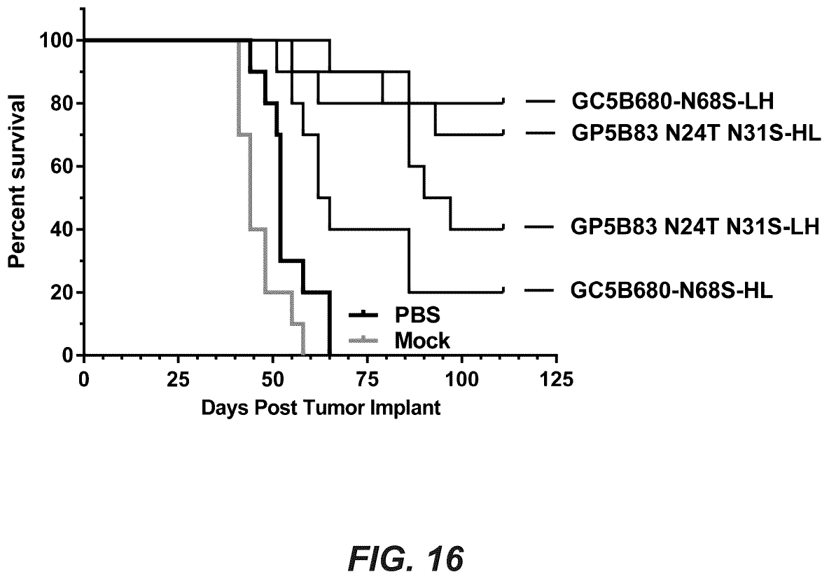

All four GPRC5D-directed CAR-T constructs elicited statistically significant % ILS at the 1×10⁶ dose level as compared with PBS and Mock CAR-T controls, as discussed below and summarized in Table 8 and FIG. 16. Animals surviving by day 111 were considered CRs.

TABLE 8
Summary of GPRC5D-directed CAR-T Efficacy on Disseminated
H929 Human MM Xenografts in NSG Mice

| | | | Median | | CRs |
|---|---|---|---|---|---|
| | CAR+ | Total | Survival | | (n of |
| Construct | T cells | T cells | Days | % ILS | group) |
| PBS | — | — | 52 | — | 0/10 |
| Mock | — | 37.1 × 10⁶ | 44 | — | 0/10 |
| GC5B680-N68S-LH | 1 × 10⁶ | 7.42 × 10⁶ | >111 | >113.5% | 8/10 |
| GC5B680-N68S-HL | 1 × 10⁶ | 7.42 × 10⁶ | 63.5 | 22.1% | 2/10 |
| GP5B83 N24T | 1 × 10⁶ | 7.42 × 10⁶ | 93.5 | 79.8% | 4/10 |
| N31S-LH | | | | | |
| GP5B83 N24T | 1 × 10⁶ | 7.42 × 10⁶ | >111 | >113.5% | 7/10 |
| N31S-HL | | | | | |

p < 0.05 vs. PBS and Mock CAR-T controls except where noted as not significant (ns).
CR = complete response

Adverse clinical signs associated with progressive tumor disease burden were observed in the PBS-treated control group beginning on day 52 post-tumor engraftment, whereas clinical signs of graft versus host disease developed in the Mock transduced or GPRC5D CAR-T treated groups beginning on day 48 post-tumor engraftment.

The teachings of all patents, published applications and references cited herein are incorporated by reference in their entirety for all purposes.

While example embodiments have been particularly shown and described, it will be understood by those skilled in the art that various changes in form and details may be made therein without departing from the scope of the embodiments encompassed by the appended claims.

1-29. (canceled)

30. An isolated nucleic acid molecule encoding a chimeric antigen receptor (CAR), comprising an extracellular antigen-binding domain, a transmembrane domain and an intracellular signaling domain, wherein the extracellular antigen-binding domain comprises:
a heavy chain CDR1 having the amino acid sequence of SEQ ID NO: 66, a heavy chain CDR2 having the amino acid sequence of SEQ ID NO: 67, and a heavy chain CDR3 having the amino acid sequence of SEQ ID NO: 68, and further comprises a light chain CDR1 having the amino acid sequence of SEQ ID NO: 69, a light chain CDR2 having the amino acid sequence of SEQ ID NO: 70, and a light chain CDR3 having the amino acid sequence of SEQ ID NO: 71;
a heavy chain CDR1 having the amino acid sequence of SEQ ID NO: 58, a heavy chain CDR2 having the amino acid sequence of SEQ ID NO: 59, and a heavy chain CDR3 having the amino acid sequence of SEQ ID NO: 60, and further comprises a light chain CDR1 having the amino acid sequence of SEQ ID NO: 61, a light chain CDR2 having the amino acid sequence of SEQ ID NO: 62, and a light chain CDR3 having the amino acid sequence of SEQ ID NO: 63;
a heavy chain CDR1 having the amino acid sequence of SEQ ID NO: 39, a heavy chain CDR2 having the amino acid sequence of SEQ ID NO: 40, and a heavy chain CDR3 having the amino acid sequence of SEQ ID NO: 41, and further comprises a light chain CDR1 having the amino acid sequence of SEQ ID NO: 48, a light chain CDR2 having the amino acid sequence of SEQ ID NO: 49, and a light chain CDR3 having the amino acid sequence of SEQ ID NO: 50;
a heavy chain CDR1 having the amino acid sequence of SEQ ID NO: 42, a heavy chain CDR2 having the amino acid sequence of SEQ ID NO: 43, and a heavy chain CDR3 having the amino acid sequence of SEQ ID NO: 44, and further comprises a light chain CDR1 having the amino acid sequence of SEQ ID NO: 51, a light chain CDR2 having the amino acid sequence of SEQ ID NO: 52, and a light chain CDR3 having the amino acid sequence of SEQ ID NO: 53; or
a heavy chain CDR1 having the amino acid sequence of SEQ ID NO: 45, a heavy chain CDR2 having the amino acid sequence of SEQ ID NO: 46, and a heavy chain CDR3 having the amino acid sequence of SEQ ID NO: 47, and further comprises a light chain CDR1 having the amino acid sequence of SEQ ID NO: 54, a light chain CDR2 having the amino acid sequence of SEQ ID NO: 55, and a light chain CDR3 having the amino acid sequence of SEQ ID NO: 56;
wherein the extracellular antigen-binding domain binds the anti-G protein receptor family C group 5 member D (GPRC5D) antigen.

31. The isolated nucleic acid molecule of claim 30, wherein the nucleic acid molecule comprises a nucleic acid sequence selected from the group consisting of SEQ ID NOS: 90, 89, 87, 88, 33, 34, 35, 36, 37, and 38.

32. The isolated nucleic acid molecule of claim 30, wherein the nucleic acid molecule comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of SEQ ID NOS: 90, 89, 87, 88, 33, 34, 35, 36, 37, and 38.

33. A vector comprising the nucleic acid molecule of claim 30.

34. A cell expressing the nucleic acid molecule of claim 30.

35-45. (canceled)
